(19) United States
(12) Patent Application Publication
(10) Pub. No.: US 2003/0199072 A1
(43) Pub. Date: Oct. 23, 2003

(54) Crystal and structure of a thermostable glycosol hydrolase and use thereof, and modified proteins

(75) Inventors: Susan J. Crennell, Bath (GB); Eva M.N. Karlsson, Lund (SE); Gudmundur O. Hreggvidsson, Reykjavik (IS); Jakob K. Kristjansson, Gardabaer (IS); Arnthor Aevarsson, Hveragerdi (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Prokaria, Itd., Reykjavik (IS)

(21) Appl. No.: 10/294,444

(22) Filed: Nov. 14, 2002

(30) Foreign Application Priority Data
Apr. 19, 2002 (IS) 6353

(51) Int. Cl. G06F 19/00; G01N 33/48; G01N 33/50; G01N 31/00; C12N 9/24

(52) U.S. Cl. 435/200; 702/19

(57) ABSTRACT

The crystal of a hyperthermostable cellulase from Rhodothermus marinus and the three-dimensional structure of the enzyme are provided. The invention further provides procedures for the identification of structural features that are important for thermostability of the enzyme. Methods based thereon to rationally modify proteins structurally related to R. marinus are disclosed, in particular, methods for increased thermostability are provided. Modified proteins are provided, including modified variants of cellulase from Trichoderma reesei.

### RELATED APPLICATION

This application claims priority to Icelandic patent application No. 6353 filed on Apr. 19, 2002, the entire contents of which are hereby incorporated by reference.

### BACKGROUND OF THE INVENTION

Cellulases are enzymes that catalyse the hydrolysis of cellulose into smaller oligosaccharides. Cellulose, a polysaccharide consisting of β-1,4-linked glucopyranose units, is the major component of plant cell walls and consequently one of the most abundant polysaccharides in nature. Microorganisms have developed a comprehensive system for enzymatic breakdown of this ubiquitous carbon source, a subject of much interest in the biotechnology industry. For example, extensive research is devoted to the development of cellulases for the production of ethanol from biomass. This research includes improvements of enzymes from microorganisms such as the filamentous fungus Trichoderma reesei (Mielenz 2001, Fowler & Mitchinson 2001, Mitchinson & Wendt 2001).

Although cellulose from terrestrial plants is the most extensively studied, other sources are also available (e.g., algae, lichens and fungi and bacteria). The ability to hydrolyse this substrate into smaller components for use as carbon and energy source is, therefore, common among microorganisms isolated from many different environments. The thermophilic bacterium Rhodothermus marinus produces a hyperthermostable cellulase, with a temperature optimum for activity exceeding 90° C.

Because of their broad use in industrial applications, cellulases with new and improved properties are highly desirable to improve existing industrial processes and for use in new applications. Desirable improvements include increased specific activity and increased thermostability (Mielenz 2001). Certain insights into stability can be gained from sequence comparisons of enzymes with different stability. For example, sequence comparisons of closely related cellulases have identified positions in Trichoderma reesei cellulase and related cellulases, which are important for the stability of the enzymes (Fowler & Mitchinson 2001, Mitchinson & Wendt 2001). Rational modifications based on structural determination and analysis of the three-dimensional structures of cellulases can also provide new and improved cellulases. The structural analysis of homologous cellulases from thermophiles and mesophiles may in particular provide information for modifications of cellulases in order to improve thermostability. The three-dimensional structures of two family 12 enzymes have been solved by others, CelB from Streptomyces lividans (Sulzenbacher et al., 1997), and Cel12A from Trichoderma reesei (Sandgren et al., 2001; abbreviated here also to TrCel12A). A high degree of structural similarity between these enzymes and the family 11 xylanases, the other family in the GH-C clan, was confirmed (Sulzenbacher et al., 1997). A structure of a thermophilic glycosyl hydrolase family 12 enzyme would be of much interest as it could provide valuable insight into the features that confer thermostability, and could direct engineering of modified proteins with increased thermostability.

### SUMMARY OF THE INVENTION

The present invention provides the first three-dimensional structure of the catalytic module of a thermostable representative from glycosyl hydrolase family 12. Comparison with cellulases from the two mesophiles allows the identification of features potentially conferring thermostability, whilst a comparison with the structures of the thermostable family 11 xylanases gives an indication of the prevalence of the proposed thermostability features within the GH-C clan. The structure of a hyperthermostable cellulase provided by the invention is the first structure of a thermostable cellulase. The analysis of the structure together with previously known structures of much less thermostable proteins provides valuable information and insight into the features contributing to thermostability in this important family of enzymes. Rational modifications based on this information or using the methods provided by the invention can be used to improve thermostability in other members of this protein family.

A first aspect of the invention provides a crystallizable composition of a thermostable clan C glycosyl hydrolase that includes family 12 glycosyl hydrolases. Preferably, the composition comprises a substantially pure protein having at least 50% amino acid sequence identity with the amino acid sequence shown in SEQ ID NO: 1, such as at least 60% sequence identity, including at least 70%, or at least 75% sequence identity, and in preferable embodiments at least 80% sequence identity, for example such as 90% sequence identity or at least 95% sequence identity, or essentially having the same sequence as shown in SEQ ID NO: 1; or a substantial part thereof, e.g., a functional part such that the protein retains glycosyl hydrolase activity.

The term “crystallisable composition” refers generally to a composition comprising a protein in a suitable liquid medium that will allow the protein to crystallize under suitable physical conditions.

In a related aspect of the invention, a crystallized molecule or molecular complex is provided comprising a protein such as described above. The crystallized molecule or molecular complex can preferably comprise a glycosyl hydrolase, such as in particular a thermophilic glycosyl hydrolase. In preferred embodiments, the crystallized molecule or molecular complex comprises a thermostable family 12 glycosyl hydrolase, which includes a family 12 glycosyl hydrolase obtainable from Rhodotermus marinus. In one embodiment, the crystallized molecule or molecular complex comprises a protein having a fβ-jelly roll fold. In a preferred embodiment the crystal of the crystallized molecule or molecular complex is characterized by a space group P2₁2₁2₁ and can further be characterized by unit cell dimensions of a=56.1 Å, b=67.8 Å, and c=132.3 Å.

In a further aspect the invention encompasses crystallized molecules or molecular complexes, in particular clan C cellulases, having a crystal structure that comprises structural entities that can be independently superimposed on reference structural entities within the structure defined by the structural coordinates of the crystallized Cel12A and as set forth in FIGS. 6A-PPP herein, such that the root mean square deviation of Cα atoms being superimposed is less than 0.8 Å or preferably less than 0.7 Å, such as less than 0.6 Å, the reference entities comprising (i) residues 18-26, (ii) residues 31-37, (iii) residues 56-64, (iv) residues 84-95, (v) residues 99-112, (vi) residues 122-142, (vii) residues 149-157, (viii) residues 161-173, (ix) residues 196-210, (x) residues 215-224 of the protein structure defined by said coordinates. In other words, the crystallized molecules or molecular complexes comprised herein have substantially similar structures to the crystallized Cel12A, in the above structurally defined regions. However, they may have less well-defined connecting regions (e.g., loops) in between these defined regions.

The term “structural entity” in this context refers to one or more sequence segments of a protein, which lie in close proximity and are connected in space, by a covalent chemical bond and/or another interactive force (e.g., ionic bond, dipole, dipole interaction, hydrogen bond); the structural entity thus comprises all or part of one or more structural motifs such as an α-helix or β-sheet.

In certain embodiments, the crystallized molecule or molecular complex of the invention comprises a polypeptide having a structure that can be superimposed on the whole protein structure defined by the structural coordinates set forth in FIGS. 6A-PPP such that the root mean square deviation of the Cα atoms of said polypeptide from the Cα atoms of said protein structure is less than 1.0 Å, such as less than 0.9 Å and preferably less than 0.8 Å. In a useful embodiment, the crystal effectively diffracts x-rays to a resolution sufficient for determination of the three-dimensional atomic coordinates, preferably the crystal diffracts x-rays to a resolution greater than 3.0 Å, more preferably greater than 2.5 Å, and even more preferably to a higher resolution than 1.8 Å.

The present invention provides a three-dimensional structure of a clan C glycosyl hydrolase, which is in certain embodiments a family 12 glycosyl hydrolase, and is the first detailed structure of a thermostable cellulase. In one aspect, the invention is a machine-readable data storage medium containing data defining the three-dimensional atomic structure of a crystallized protein or crystallized protein complex such as described above, including a crystallized protein that is a clan C glycosyl hydrolase, such as a family 12 glycosyl hydrolase, such as in particular the cellulase Cel12A obtainable from Rhodothermus marinus. In a particular embodiment, said data essentially defines the protein structure represented by the structure coordinates set forth in FIGS. 6A-PPP, e.g., by being encoded with said structure coordinates, or mathematically related coordinates defining essentially the same structure as said coordinates. The term “mathematically related coordinates” refers to coordinates that have different numerical values, e.g., they could refer to a different point of origin, but can be transformed by a mathematical relation to the coordinates to which they relate to, such as, for example, by translation or a symmetry operation. Data that essentially defines said structure could also be represented by other types of data such as by dihedral angles and general geometrical restraints. The machine-readable data storage medium is any suitable data storage medium, many of which are well known in the art, such as a hard disk, magnetic tape or disk, or an optical disk, flash memory, or the like, readable by a computer equipped for reading such data storage medium.

It is an object of the invention to provide for homology modelling (also known as comparative modelling, Sanches & Sali 1997; Forster 2002) of clan C glycosyl hydrolases including family 12 glycosyl hydrolases and structurally related proteins. In one aspect of the invention, atomic coordinates are provided that can be used to construct a model of a homologous protein. In one embodiment, a method is provided for modelling the structure of a first protein with at least 25% amino acid sequence identity to the sequence set forth in SEQ ID NO: 1 and preferably higher sequence identity such as at least 40%, or at least 50%, and more preferably at least 60%, including at least 75% or at leas 80% such as, e.g., at least 90% or at least 95% sequence identity to the sequence set forth in SEQ ID NO: 1; comprising aligning the sequence of said first protein with the sequence of a reference crystallized protein of the invention with determined crystal structure (preferably with SEQ ID NO: 1) and incorporating the sequence of said first protein into the structure of said reference protein, thereby creating a structural model of said first protein. Said structural model can consist of a partial structure including only fragments corresponding to structurally conserved regions. Said structural model can further be subjected to energy minimization to obtain an energy-minimized structural model. Energy minimization of a molecular system can be performed using some of the methods available employing minimization algorithms, based on molecular potential energy as a function of atomic positions, and optionally combined with molecular dynamics such as in a “simulated annealing” scheme (Forster, 2002). Regions of said energy-minimized model can be re-modeled where stereochemistry restraints are violated to obtain structure coordinates of an improved structural model of said first protein. The procedure can be repeated for additional rounds of energy minimization and remodelling. Optionally, regions of said structural model, such as structurally variable regions between structurally conserved regions, can be further modelled using information of other predetermined structure models. Geometrical restraints can be used in the modelling scheme in different ways to generate models that best satisfy the restraints. Geometrical restraints, which include, for example, limits on distances between atom pairs and ranges of dihedral angles, are often included in energy minimization and molecular dynamics procedures (Havel & Snow 1991; Sali & Blundell 1993; Forster 2002).

In a related aspect, a method is provided for determining a protein structure of a first protein from crystallographic protein structure data that has insufficient phase information for a structure determination, comprising determining the phase information for said first protein with molecular replacement methods based on an obtained structure of a crystallized protein of the present invention; and determining the protein structure by use of the initial structure data and the obtained phase information. It follows that said first protein should be structurally related to said crystallized protein, e.g., having a sequence identity of at least 30%, such at least 50% or higher, e.g., at least 60%, and preferably at least 70%, including at least 80% sequence identity to said crystallized protein. This method will be particularly useful in cases where crystals have been obrtained for a first protein and crystallographic data obtained, but where crystals of heavy atom derivatives of said first protein have not been obtained and/or refraction data for such derivative crystals are not of suufucient quality to determine the phase of the refraction data of the non-derivatized crystals.

A further aspect of the invention provides a method for predicting the structure of a first protein comprising: obtaining a protein structure of a second protein from the same protein family according to the invention; and predicting the structure of first protein with homology modeling based on the structure of said structure and of the relevant sequences.

It is a further object of the invention to provide structural models of family 12 glycosyl hydrolases that can be used for rational protein design in order to change properties of an enzyme through changes made in the amino acid sequence. In preferred embodiments of the invention, the amino acid changes that are made increase thermostability.

It will be appreciated that the present invention provides a method for modifying in a structurally defined region of a first protein that is related to a crystallized protein of the invention said first protein preferably being a clan C glycoyl hydrolase, including a family 12 glycosyl hydrolase, the method comprising the steps of: obtaining a first amino acid sequence of said first protein and a nucleic acid encoding said sequence, and aligning said first sequence with the sequence of said crystallized protein; selecting a region in said first sequence that aligns with a structurally defined region in said crystallized protein, and changing the nucleotide sequence of said nucleic acid in the region that encodes for said region in said first sequence to exchange, add and/or subtract one or more amino acid residues in said region of said first protein; and expressing said modified first protein in a suitable expression system.

The term “structurally defined region” refers to a part of a protein that either has a defined structure as determined by structure determination methods such as of the present invention, or is postulated to have a defined structure based on sequence alignment with a part of a protein with determined structure or other modelling techniques.

In useful embodiments, said modification comprises one or more of the above-mentioned features that contribute to thermostability of R. marinus cellulase. Preferably, the modification of said method comprises one or more modifications from the group consisting of:

having an Arg, Lys or His residue at one position and Asp or Glu residue at a second position, which positions are at sequence locations that align with Glu4 and Arg47 of SEQ ID NO: 1, respectively;

having an Arg, Lys or His residue at one position and Asp or Glu residue at a second position, which positions are at sequence locations that align with Arg8 and Glu29 of SEQ ID NO: 1, respectively;

having an Arg, Lys or His residue at one position and Asp or Glu residue at a second position, which positions are at sequence locations that align with Asp10 and Arg12 of SEQ ID NO: 1, respectively;

having an Arg, Lys or His residue at one position and Asp or Glu residue at a second position, which positions are at sequence locations that align with Asp10 and Arg20 of SEQ ID NO: 1, respectively;

having an Arg, Lys or His residue at one position and Asp or Glu residue at a second position, which positions are at sequence locations that align with Asp13 and Arg20 of SEQ ID NO: 1, respectively;

having an Arg, Lys or His residue at one position and Asp or Glu residue at a second position, which positions are at sequence locations that align with Glu35 and Arg216 of SEQ ID NO: 1, respectively;

having an Arg, Lys or His residue at one position and Asp or Glu residue at a second position, which positions are at sequence locations that align with Arg47 and Asp49 of SEQ ID NO: 1, respectively;

having an Arg, Lys or His residue at one position and Asp or Glu residue at a second position, which positions are at sequence locations that align with Asp51 and Arg100 of SEQ ID NO: 1, respectively;

having an Arg, Lys or His residue at one position and Asp or Glu residue at a second position, which positions are at sequence locations that align with His67 and Glu203 of SEQ ID NO: 1, respectively;

having an Arg, Lys or His residue at one position and Asp or Glu residue at a second position, which positions are at sequence locations that align with Arg79 and Glu83 of SEQ ID NO: 1, respectively;

having an Arg, Lys or His residue at one position and Asp or Glu residue at a second position, which positions are at sequence locations that align with Arg80 and Glu83 of SEQ ID NO: 1, respectively;

having an Arg, Lys or His residue at one position and Asp or Glu residue at a second position, which positions are at sequence locations that align with Arg80 and Glu196 of SEQ ID NO: 1, respectively;

having an Arg, Lys or His residue at one position and Asp or Glu residue at a second position, which positions are at sequence locations that align with Asp86 and Arg88 of SEQ ID NO: 1, respectively;

having an Arg, Lys or His residue at one position and Asp or Glu residue at a second position, which positions are at sequence locations that align with Arg88 and Glu177 of SEQ ID NO: 1, respectively;

having an Arg, Lys or His residue at one position and Asp or Glu residue at a second position, which positions are at sequence locations that align with Arg88 and Asp179 of SEQ ID NO: 1, respectively;

having an Arg, Lys or His residue at one position and Asp or Glu residue at a second position, which positions are at sequence locations that align with Arg100 and Glu210 of SEQ ID NO: 1, respectively;

having an Arg, Lys or His residue at one position and Asp or Glu residue at a second position, which positions are at sequence locations that align with Arg141 and Glu153 of SEQ ID NO: 1, respectively;

having an Arg, Lys or His residue at one position and Asp or Glu residue at a second position, which positions are at sequence locations that align with Glu153 and Arg167 of SEQ ID NO: 1, respectively;

having an Arg, Lys or His residue at one position and Asp or Glu residue at a second position, which positions are at sequence locations that align with Asp179 and Lys181 of SEQ ID NO: 1, respectively;

having an Arg, Lys or His residue at one position and Asp or Glu residue at a second position, which positions are at sequence locations that align with Lys181 and Asp185 of SEQ ID NO: 1, respectively;

having an Arg, Lys or His residue at one position and Asp or Glu residue at a second position, which positions are at sequence locations that align with Asp186 and Arg190 of SEQ ID NO: 1, respectively;

having an Arg, Lys or His residue at one position and Asp or Glu residue at a second position, which positions are at sequence locations that align with Arg194 and Glu196 of SEQ ID NO: 1, respectively;

having an Arg, Lys or His residue at one position and Asp or Glu residue at a second position, which positions are at sequence locations that align with Arg216 and Asp219 of SEQ ID NO: 1, respectively.

In one embodiment, the modification of the method comprises having a Gln, Asn, Arg, Lys, His, Asp or Glu residue at the sequence location that aligns with Gln82 of SEQ ID NO: 1; while another embodiment the modification comprises having an Asp or Glu residue at the sequence location that aligns with Glu39 of SEQ ID NO: 1 and an N-terminal residue at the sequence location that aligns with Thr2 of SEQ ID NO: 1.

The method comprises in yet a further embodiment a modification stabilizing a helix corresponding to residues 180-191 of SEQ ID NO: 1 by having one or both modifications from the group consisting of: having an Arg, Lys or His residue at the sequence location that aligns with Gln82 of SEQ ID NO: 1; and having an Asp or Glu residue at the sequence location that aligns with Asp179 of SEQ ID NO: 1.

Also provided are proteins modified by said method.

It is yet a further object of the invention to provide for an variant clan C glycosyl hydrolase such as a family 12 glycosyl hydrolase or a related enzyme, wherein one or more amino acids are exchanged, added or deleted in order to change properties of the enzyme. In particularly advantageous embodiments the modifications of such proteins confer increased thermostability to the proteins. Useful embodiments include modified variants of cellulase obtainable from a Trichoderma species such as Trichoderma reseei. Such modifications preferably comprise one or more of the above-mentioned substitutions, such as to increase the number of ionic pairs (e.g., create an ionic pair found in R. marinus cellulase but not in mesophilic members of family 12 Glycosyl hydrolases), or to engineer a more rigid loop region corresponding approximately in location to residues 155-165 in SEQ ID NO: 1.

In useful embodiments, variant clan C glycosyl hydrolases or related enzymes are provided wherein one or more amino acids are exchanged, added or deleted at positions corresponding to positions 4, 8, 10, 12, 13, 20, 29, 35, 47, 49, 51, 79, 80, 83, 86, 88, 100, 138, 141, 153, 155-165, 167, 177, 179, 181, 185, 186, 190, 194, 196, 210, 216 and 219 in the family 12 glycosyl hydrolase Cel12A from R. marinus (SEQ ID NO: 1).

In particular embodiments of the invention, the proteins provided are truncated by one or more N-terminal residues of the corresponding wild-type enzymes. Such truncation modification can significantly improve the stability and even increase the activity of the proteins of the invention, such as of family 12 glycosyl hydrolases, as disclosed in detail in applicant's co-pending application WO 01/96382. Such a truncation will preferably remove all or part of the N-terminal portion corresponding to an N-terminal hydrophobic domain and/or linker domain. Such domains essentially comprise residues 1-17 and 18-37 respectively in the wild-type cellulase from R. marinus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation of the structure of R. marinus Cel12A, with sheet A black and sheet B grey. Individual strands are labeled according to their position within the sheets. The HEPES molecule bound in the active site is shown in a ball-and-stick representation.

FIGS. 2A and 2B depict a structure-based sequence alignment of family 12 cellulase sequences (drawn using ALSCRIPT (Barton 1993)). Structures have been determined for the top three sequences: Cel12A, S. lividans CelB2 (PDB:2NLR, Sulzenbacher et al., 1999) and T. reesei TrCel12A (PDB: 1H8V, Sandgren et al., 2001), these are followed by representative members of the Erwinia (Erwinia carotovora Genbank AAA24817, 31% identity), Aspergillus (Aspergillus kawachii, Genbank BAA02297, 30%), Thermatoga (Thermatoga neapolitana celB Genbank AAC95060, 31%) families and Pyrococcus furiosus (Genbank AAD54602, 31% identity). The secondary structure of Cel12A, shaded and annotated to match FIG. 1, is drawn above each block of sequence, and residues implicated in the active site are indicated by subsite numbers underneath. The two catalytic residues are marked with triangles. Shading of the sequences denotes conservation, calculated using ALSCRIPT within the sequences with structures and across the whole family. Light grey shading denotes similarity across the sequences (in both blocks), dark grey being identical across the three structures, and black with white letters being conserved across all family 12 cellulase sequences. The “mobile loop” in CelB2 is outlined.

FIG. 3 depicts two perpendicular views of the HEPES molecule (black bonds) together with the catalytic residues with which it interacts, superimposed on the fluorocellosyl moiety (grey bonds) bound in the CelB2 structure (aligned using the catalytic residues). The electron density of a difference F₀-F_{c} map in the absence of HEPES is drawn at 3≐ in chicken-wire representation and covers the HEPES, but also extends towards Glu124. This may be an indication of multiple HEPES conformations not resolved at 1.8 Å.

FIGS. 4A and 4B show schematic representations of the active sites of A) Cel12A with HEPES bound and B) CelB2 with 2-deoxy-2-fluorocellotrioside bound in the central-1 subsite. The amino acids that interact with cellulose are drawn with orange bonds, the inhibitor is shown in black (with the sugars in the -2 and -3 subsites of CelB2 drawn smaller for clarity), and hydrogen bonds as dotted lines. The “cord” loop is coloured pale grey.

FIGS. 5A-C are schematic representations of the “Mobile Loop” region in the active site of the three structures, A) S. lividans CelB2, B) T. reesei TrCel12A and C) Cel12A. Ball-and-stick representations of residues within the loop itself have yellow bonds, others are grey, including the two catalytic Glutamic acid residues. Molecules bound in the active site have black bonds. Hydrogen bonds are drawn as dotted green lines.

FIGS. 6A-PPP show the structure coordinates (Protein Data Bank file format) of the crystal structure of Cel12A from Rhodothermus marinus.

### DETAILED DESCRIPTION OF THE INVENTION

The enzymes that hydrolyse the cellulose polymer (i.e., cellulases) are traditionally divided into two major groups: endoglucanases (EC 3.2.1.4) and cellobiohydrolases (or exoglucanases) (EC 3.2.1.91), both attacking β-1,4-glycosidic bonds. The endoglucanases catalyse random cleavage of internal bonds in the cellulose chain, while cellobiohydrolases attack the chain ends and release cellobiose. A third group of enzymes related to cellulose hydrolysis are the β-glucosidases (EC 3.2.1.21), but these enzymes are only active on cello-oligosaccharides and cellobiose, and do not use cellulose as substrate.

Cellulases, as well as other glycosyl hydrolysing enzymes, often display a modular design, forming discrete functioning units connected by recognisable linker sequences. The most common type of auxiliary modules are carbohydrate binding modules (CBM). The catalytic modules of glycosyl hydrolases are classified in a system based on primary sequence similarities (Henrissat, 1991; Henrissat and Bairoch, 1993), which currently consists of more than 80 protein families (see, e.g., Coutinho and Henrissat, 1999). Members of the different families can display differences in both architecture and substrate specificity. The cellulase catalytic modules are found in at least 12 of these families, (5-9, 12, 44-45, 48, 51, 61, 74), with most of the published sequences classified into families 5 and 9. Because the fold of proteins is more highly conserved than their coding or amino acid sequences, structural determinations have demonstrated structural homologous between members of some families, and these related protein families have been grouped in clans named glycosyl hydrolase (GH) clan A-K (Henrissat & Davies 1997). To date, 7 of the clans have been confirmed by 3D-structural study, and comprise 4 different folds: (β/α)₈ for GH-A, -H, and K; β-jelly roll for GH-B and GH C; 0-propeller for GH-E; and α+β for GH-I. Four of the cellulase families have been grouped into the clan-system, family 5 and 51 in GH-A, family 7 in GH-B, and family 12 in GH-C. Irrespective of family and clan affiliation, enzymatic hydrolysis of the glycosidic bond takes place via general acid catalysis requiring two critical residues, a proton donor and a nucleophile, and leads to either inversion or retention of the anomeric configuration.

The cellulase (Cel12A) from the thermophilic bacterium Rhodothermus marinus is a member of glycosyl hydrolase family 12 (Halldorsdottir et al, 1998). This enzyme consists of a single catalytic domain connected by a flexible linker to a putative signal peptide (Wicher et al., 2001), i.e., the enzyme does not have a cellulose binding module (CBM). The substrate specificity of the enzyme is typical of the family 12 enzymes, hydrolysing β-1,4- glucosidic linkages in various types of β-glucans. The enzyme is resistant to thermal inactivation (with a half-life of more than 2 h at 90° C.) and is active at high temperatures (exceeding 90° C.) (Alfredsson, et al., 1988). In terms of its thermostability, it is comparable to the cellulases from the two hyperthermophiles Pyrococcus furiosus (Bauer et al., 1999) and Thermotoga species (Liebl et al., 1996, Bok et al., 1998).

The present invention provides a crystal and the structural coordinates of a hyper-thermostable Cellulase Cel12A from Rhodothermus marinus. The invention provides analysis of the structure including methods for compariing the structure with other known structures of homologous enzymes for the identification of structural features conferring thermostability. The invention further provides methods of using the structural coordinates and/or the structural information disclosed through the structural analysis for protein design of homologous proteins.

“Cellulose” is a polysaccharide consisting of β-1,4-linked glucopyranose units.

When appearing herein on their own, “Cel12A” refers to Rhodothermus marinus cellulase Cel12A (SEQ ID NO: 1), “CelB2” refers to Streptomyces lividans cellulase CelB2, and “TrCel12A” refers to Trichoderma reesei cellulase Cel12A (SEQ ID NO: 2).

“HEPES” is N-[2-Hydroxyethyl]piperazine-N′-[2-ethanesulphonic acid] and “CMC” is carboxymethylcellulose.

The term “homologous” as used herein refers generally to sequences that share sequence similarity by virtue of common descent.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual alignment of the two sequences can be accomplished by well-known methods, for example, using a mathematical algorithm. A preferred, non-limiting example of such a mathematical algorithm is described in Karlin et al., 1993. Such an algorithm is incorporated into the various BLAST programs (version 2.0) as described in Altschul et al., 1997. When utilizing BLAST and Gapped BLAST programs, the default parameters of the respective programs (e.g., blastp, provided by the National Center for Biotechnology Information, NCBI) can be used. In one embodiment, parameters for sequence comparison can be set at score=10, wordlength=3, or can be varied.

Another preferred non-limiting example of a mathematical algorithm utilized for the alignment of sequences is the algorithm of Myers and Miller 1988. Such an algorithm is incorporated into the ALIGN program (version 2.0), which is part of the GCG sequence alignment software package (Accelrys, Cambridge, U.K.). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti 1994; and FASTA described in Pearson and Lipman 1988.

Additionally, the percent identity between two amino acid sequences can be determined using the GAP program in the GCG software package using either a Blossom 63 matrix or a PAM250 matrix, and a gap weight of 12, 10, 8, 6, or 4 and a length weight of 2, 3, or 4. Also, the percent identity between two nucleic acid sequences can be determined using the GAP program in the GCG software package, using a gap weight of 50 and a length weight of 3.

“Substantial sequence similarity” to the R. marinus Cel12A cellulase refers to polypeptides, or fragments or derivatives thereof, having at least 30% sequence identity to SEQ ID NO: 1, but preferably having at least 40% sequence identity, such ad at least 50% sequence identity to SEQ ID No: 1. The amino acid sequence of the polypeptide can be that of the naturally-occurring polypeptide or can comprise alterations therein. Polypeptides comprising alterations are referred to herein as “derivatives” of the native polypeptide. Such alterations include conservative or non-conservative amino acid substitutions and additions and deletions of one or more amino acids.

Proteins with “substantial structural similarity” to the R. marinus Cel12A cellulase refers to proteins with substantial structural similarity inferred from substantial sequence similarity or proteins with known structure having at least one or more structural entitities that can be superimposed on reference structural entities within the structure of Cel12A.

Thermostable enzymes (also referred to as “thermozymes”) are intrinsically stable and active at a high temperature, in the range of about 30-100° C., but more typically they refer to enzymes optimized for temperatures in the range of 40-100° C., in particular at high temperatures found in hot geothermal areas such as in the range of 60-100° C.

Thermostable enzymes from thermophiles and hyperthermophiles are optimally active at temperatures close to or above the optimal temperature for growth of the source organism. The molecular basis for thermal stability, as demonstrated by comparing a thermostable protein to a homologous thermolabile protein, resides in the cumulative effect of variations in the amino acid sequence. These differences can contribute to enhanced thermostability in numerous ways, such as by altering the entropy of unfolding, making hydrophobic core packing tighter, stabilizing helices and adding disulfide bridges, salt bridges and hydrogen bonds. Possible strategies to obtain enzymes with high thermal stability (for industrial applications) include screening for thermostable enzymes from thermophiles and introducing changes in the amino acid sequence, such as by directed evolution or rational design, of a relatively thermolabile protein in order to enhance thermostabilty. Suitable changes for thermostabilizing protein engineering can be provided through careful analysis of the three-dimensional structures of homologous thermostable and thermolabile proteins obtained from a thermophile and a mesophile, respectively (Chen 2001; Vieille & Zeikus 2001; Sterner & Liebl 2001; Szilagyi & Zavodszky 2000).

The term “thermophile” refers herein to any microorganism thriving at high temperature conditions, i.e., above about 45° C., while the term “mesophile” refers to microrgansims thriving at moderate temperatures such as in the range of about 12-45° C., and typically at temperatures between 12-25° C. Hyperthermophiles refer generally to thermophiles thriving at extreme temperatures, such as in the range of about 70-100° C.

Isolation and Crystallization of Cel12A Cellulase from Rhodothermus marinus.

In one aspect of the invention, a method is provided for obtaining a crystallized protein of the present invention, such as, for example, Cellulase Cel12A from Rhodothermus marinus. The method includes expressing, purifying and crystallizing said protein. Expression of selected genes or gene fragments can be conveniently performed in a suitable host, such as prokaryotic or eukaryotic cells (e.g., bacterial cells such as Escherichia coli can be utilized by cloning an appropriate expression vector such as “ATG vectors” into the cells (Aman & Brosius 1985)). The expression of the gene can be controlled by using a vector with a suitable promoter system,such as the T7 promoter (Studier et al. 1990). Alternatively, the recombinant expression vector can be transcribed and translated in vitro, for example, using T7 promoter regulatory sequences and T7 polymerase. The protein can be purified with suitable standard purification methods, such as, e.g., liquid chromatography. Columns with resins specific for an affinity purification using purification tags can be used to simplify purification. A heat-denaturation step can be effectively used as a purification step for the thermostable protein expressed in a mesophilic host such as E. coli. Purity of the protein preparations can be determined via SDS-PAGE. Protein preparations can be analyzed with different techniques to evaluate their suitability for crystallization trials and to establish conditions more suitable for the purification and crystallization of a particular protein. This includes circular dichroism to analyze stability and folding, light scattering to analyze if the protein preparation is monodisperse, analytical centrifugation to analyze molecular weight distribution or mass spectrometry techniques.

Crystallization can be performed by screening for appropriate conditions with suitable precipitation agents using a standard techniques such as hanging or sitting drop vapor diffusion (Methods in Enzymology 114, 1985; McPherson 1999; Methods in Enzymology 276, 1997; McPherson, 1990). Pre-made sparse matrix screens can conveniently be used for fast initial screening of many different conditions (Jancarik & Kim, 1991). Further screening for crystallization conditions and optimization can be done in a more systematic way for a particular precipitant (McPherson, 1999). After crystals have been obtained, conditions in the presence of a cryosolvent can be found for the subsequent freezing of the crystals at cryogenic temperatures (Watenpaugh, 1991).

The present invention provides a crystalline composition of a cellulase from Rhodothermus marinus. As described in detail in Example 1, a truncated form of the protein was expressed, purified and crystallized using the hanging drop method. The specific construct of the protein serves only as an example and a range of modified or unmodified active cellulases from Rhodothermus marinus or a substantially similar protein from a related source, preferably a clan C glycosyl hydrolase including family 12 glycosyl hydrolases can be used according to the invention. Similarly, any person skilled in the art of protein crystallization having the present teaching could crystallize alternative forms of the cellulase from a variety of fragments or a full-length cellulase from a related source. A cellulase having conservative substitutions in its amino acid sequence, crystals of such a cellulase, crystallization conditions for such a cellulase and methods of using such a cellulase are also encompassed by the invention. Conservative substitutions refer herein to amino acid substitutions that replace an amino acid residue by another with similar properties, e.g., a positively charged residue exchanged for another positively charged residue (e.g., Lys for Arg), a hydrophobic residue exchanged for another hydrophobic residue (e.g., Phe for Tyr), etc.

In the example below, a catalytic module of the cellulase was crystallized at 291K by the hanging drop vapour diffusion method, using a protein concentration of 14 mg/mL. The best quality crystals were obtained in 48 h from 0.1 M HEPES, pH 7.5, 20% w/v PEG 10000 and grew to dimensions of 1.7×0.4×0.3 mm.

Methods for crystallizing crystallizable compositions and for obtaining three-dimensional structural information from such crystals are well known in the art. The enclosed illustrating example (Example 1) describes in detail how the three-dimensional structure of Cellulase Cel12A from Rhodothermus marinus was obtained. Generally, the method to obtain a three-dimensional structure from a crystallizable composition of the present invention comprises: obtaining a cystallized protein such as described above; collecting diffraction data for the obtained crystal of the candidate protein; obtaining complementary data for phase determination of the diffraction data; and determining the protein structure by use of the obtained data.

Data is collected using a suitable x-ray source such as a laboratory x-ray generator or a synchrotron x-ray source especially for multiple wavelength experiments such as MAD (Multiwavelength Anomalous Diffraction; Hendrickson, 1991). Crystal mounting and data collection using frozen crystals requires the use of cryogenic equipment installed near the laboratory generator or at the synchrotron beam line. Data can be recorded using special detectors, such as image plates or CCD (charged coupled device) detectors, and the appropriate goniostat and other equipment for the alignment and controlled movement of the crystal during data collection. Image data processing can be done with software such as Denzo (Otwinowski & Minor, Methods Enzymol., 277:307-326 (1997)) and data reduction and general crystallographic computing is suitably done with various programs including those in the CCP4 package. Data collected at single wavelength of only the native protein normally gives only amplitudes but no phase information (which is required to compute electron density map and determine the structure through interpretation of the map). Sufficient phase information has to be obtained by additional experiments.

Phase information can be obtained with any of the methods known to those skilled in the art. Methods for phase determination in the crystallography of biological macromolecules include single isomorphous replacment (SIR) or multiple isomorphous replacement (MIR), with or without anomalous scattering and MAD. These methods require the use of heavy atom derivatives of the protein, which can be obtained, for example, by soaking the protein crystals in a heavy atom compound solutions (Isomorphous Replacement and Anomalous scattering (Wolf et al., 1991) or by expression of the protein in a suitable host in the presence of selenomethionine to make selenomethionine-substituted protein. The position of the heavy atom scatterer can be found with different methods, including the use of automated programs such as SOLVE (Terwilliger & Berendzen, 1999). Refinement of heavy atom parameters and phase calculation can be done with programs such as SHARP (De La Fortelle & Bricogne, 1997) and density modification with programs such as DM (Cowtan, 1994). Phasing can also be achieved with molecular replacement using an available structure of a similar homologous protein (Rossman, 1972; Fitzgerald, 1988; Navazza, 1994). Howver, phase information obtained by any of these methods will not always be of adequate quality. Sufficient phase information will allow reliable interpretation of an electron density map computed using the phase information.

Interpretation of the electron density maps and model building can be done manually, for example, with the program O (Jones et al., 1991) or with more automated procedures (Perrakis et al., 1997). Refinement of coordinates can be performed using the program CNS (Brunger et al., 1998). Coordinates made publicly available are normally deposited in the Protein Data Bank.

The crystallographic methods and specific software mentioned here are meant to provide illustrating examples of methods and computing tools currently in use in the art, and are, therefore, not meant to be limiting. Other methods and software known to those skilled in the art can also conveniently be used for structure determination using x-ray crystallography.

Structural Analysis and Determination of Thermostabilizing Features.

The three-dimensional structure of the Cel12A cellulase from Rhodothermus marinus provided by the invention consists of two β-sheets packed against each other to form a single domain of dimensions roughly 40×40×30 Å. The structure resembles the previously determined structure of Streptomyces lividans CelB2 and Tricoderma resei Cel12A. Catalytic residues, verified by experiments in previous structures, are located in a cleft formed by one of the β-sheets.

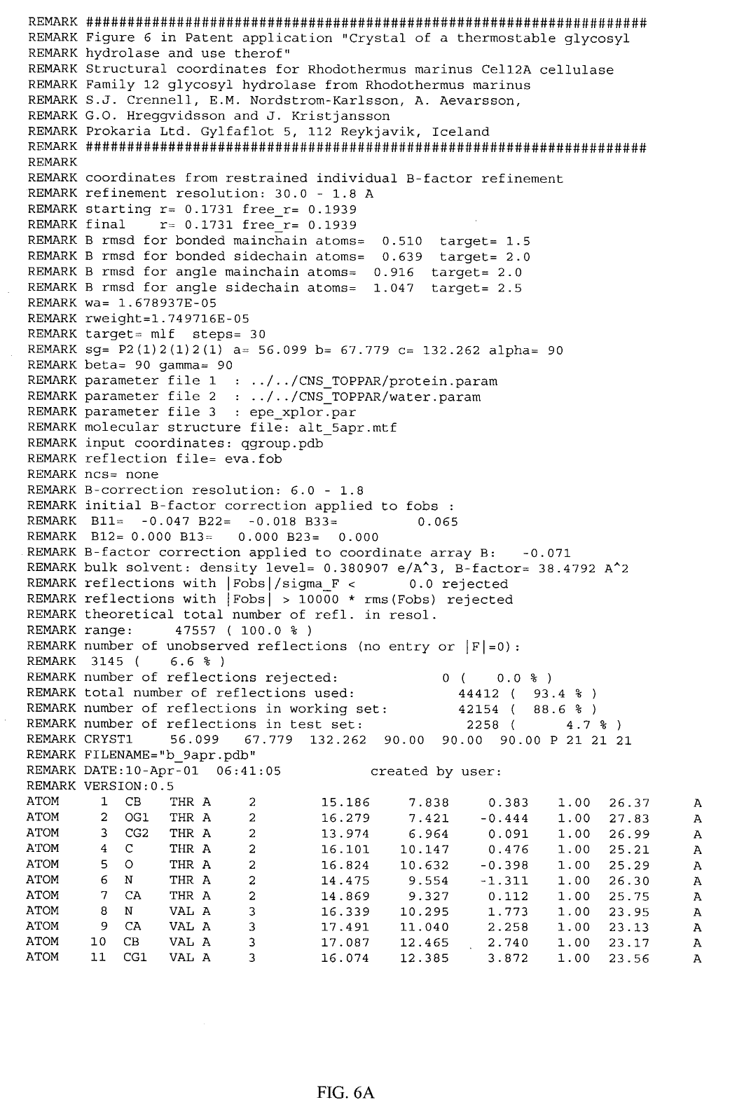

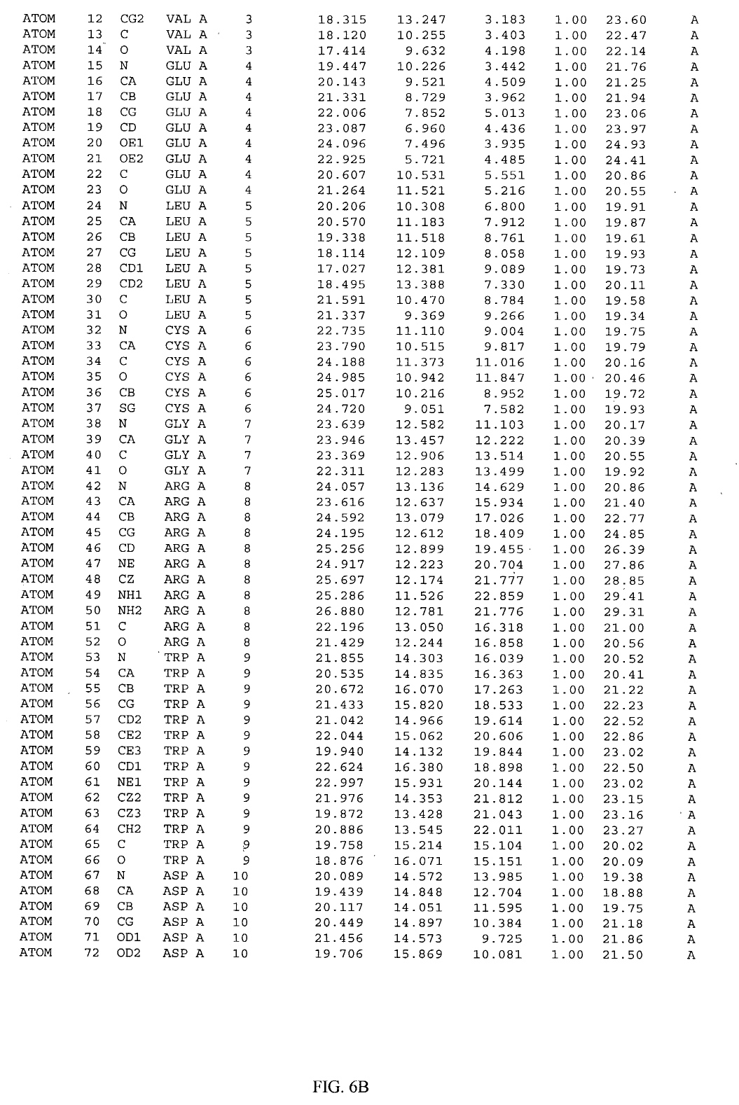

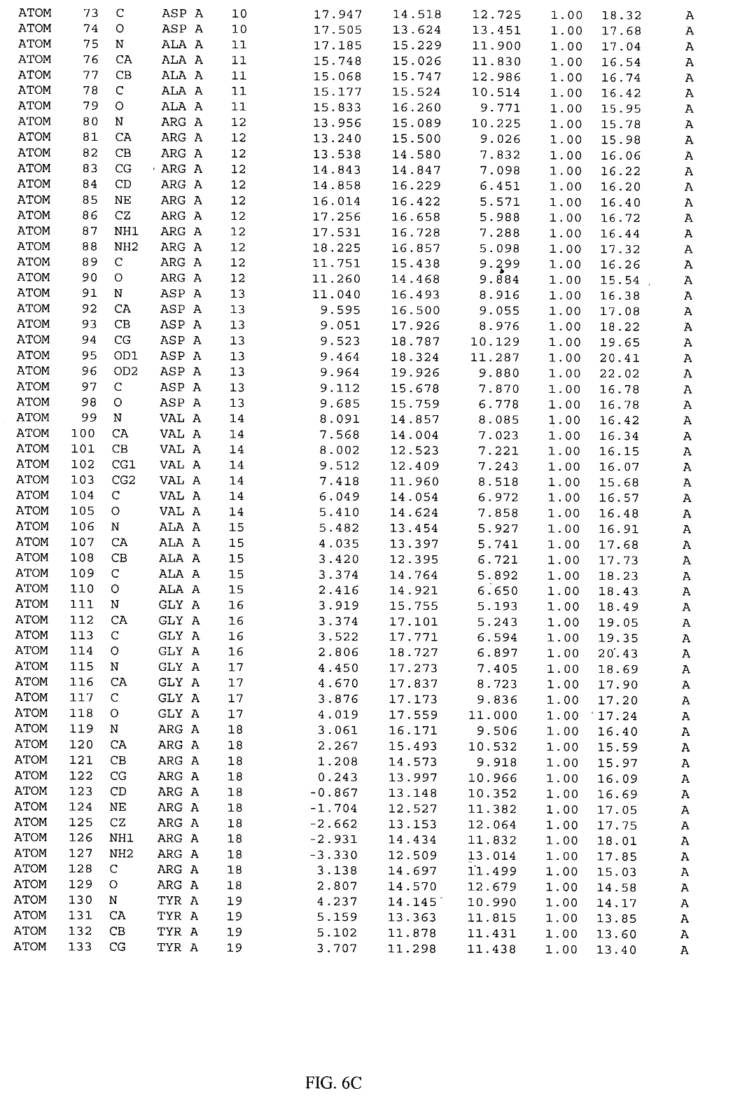

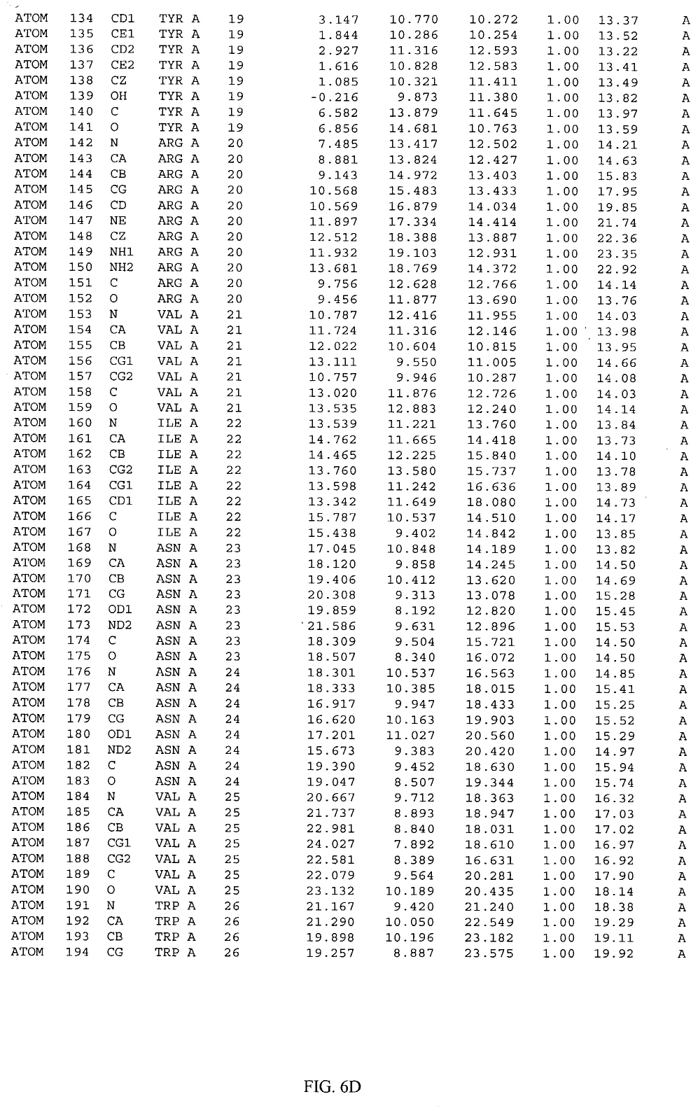

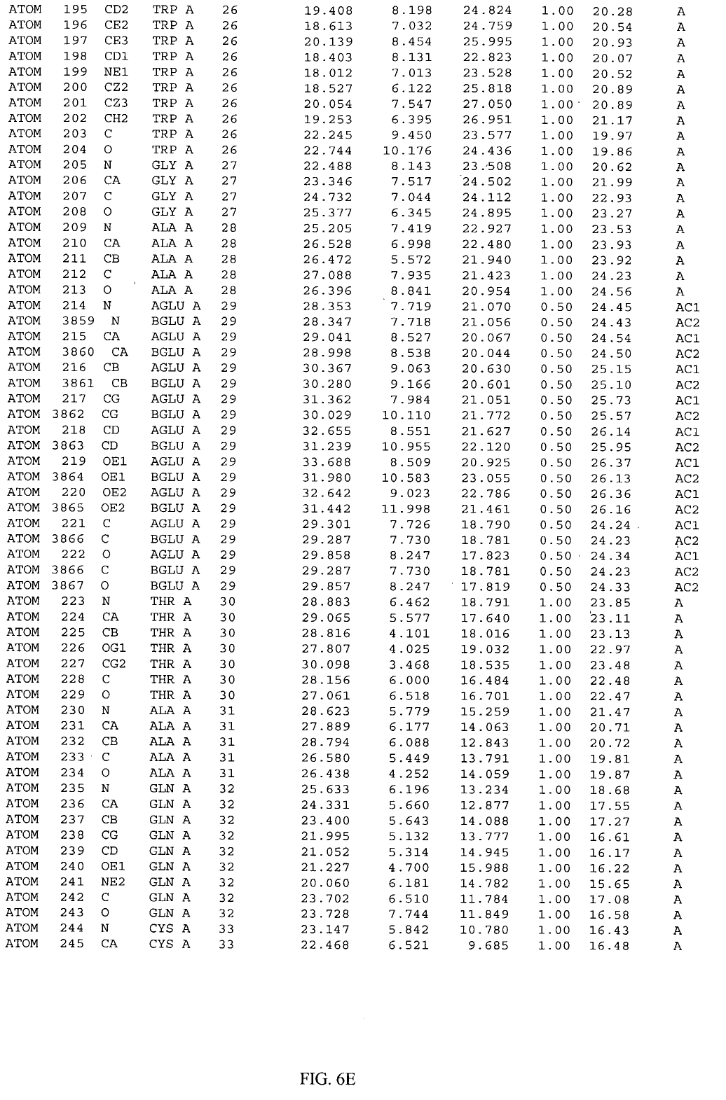

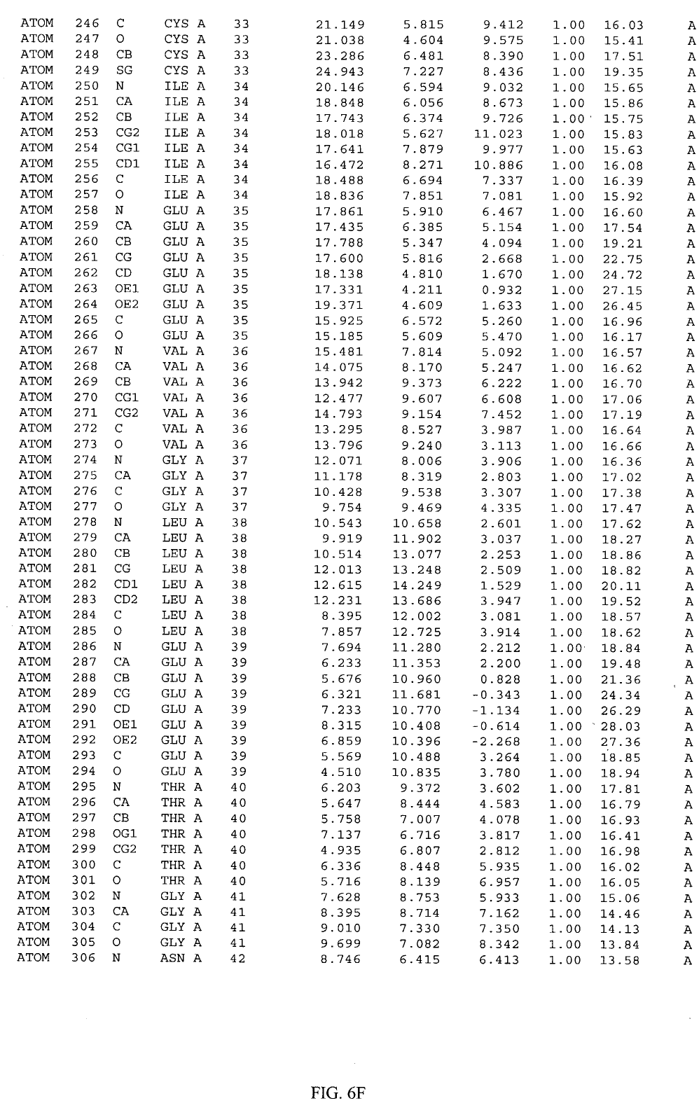

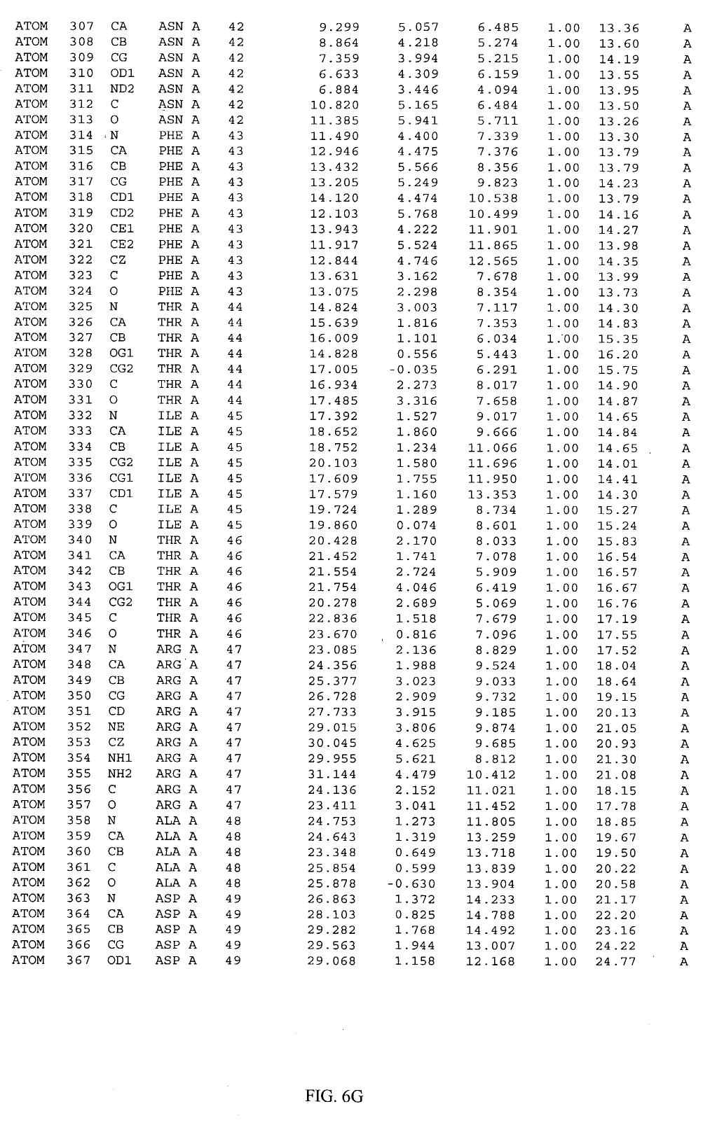

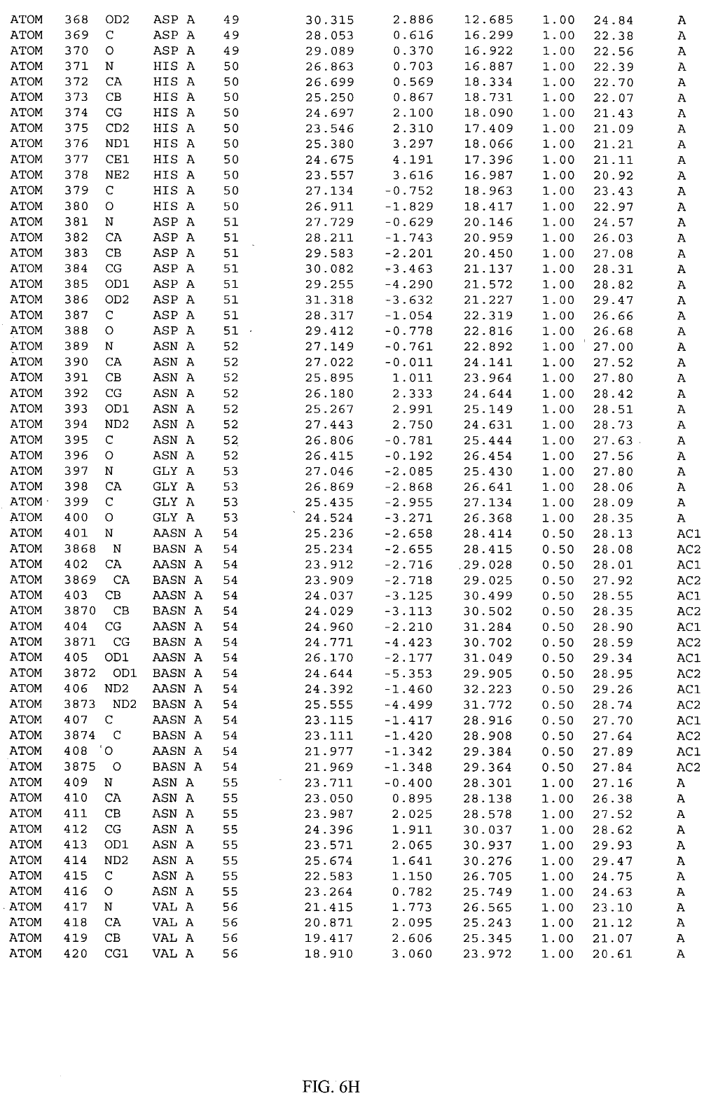

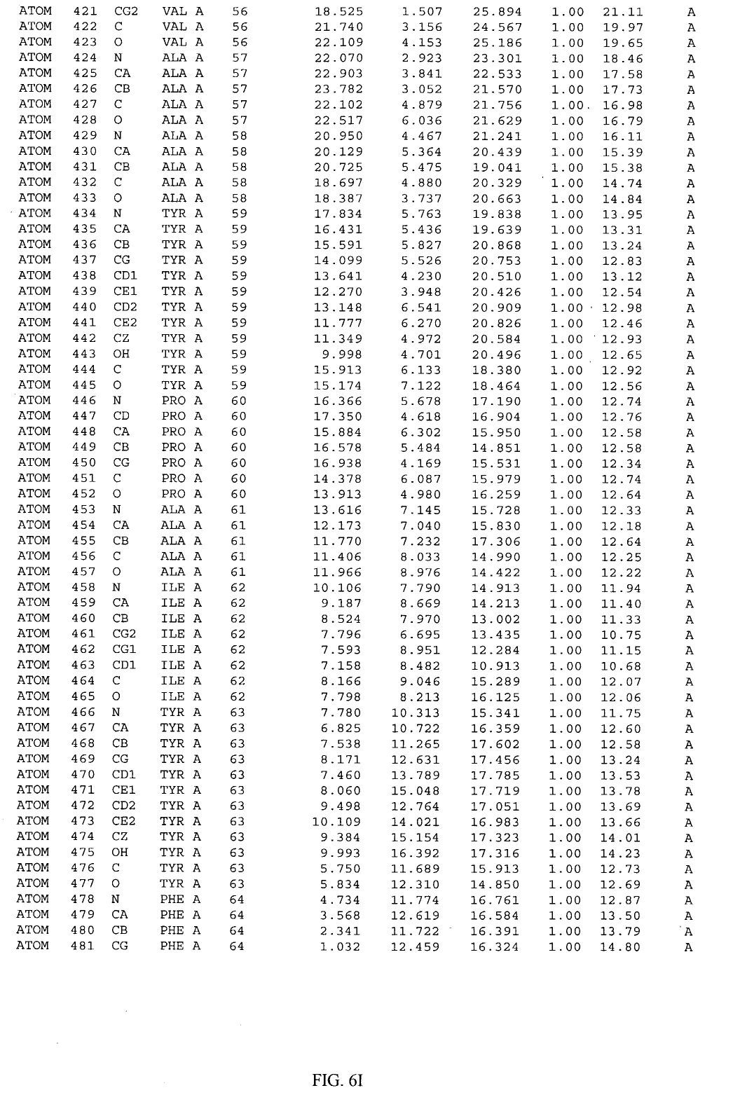

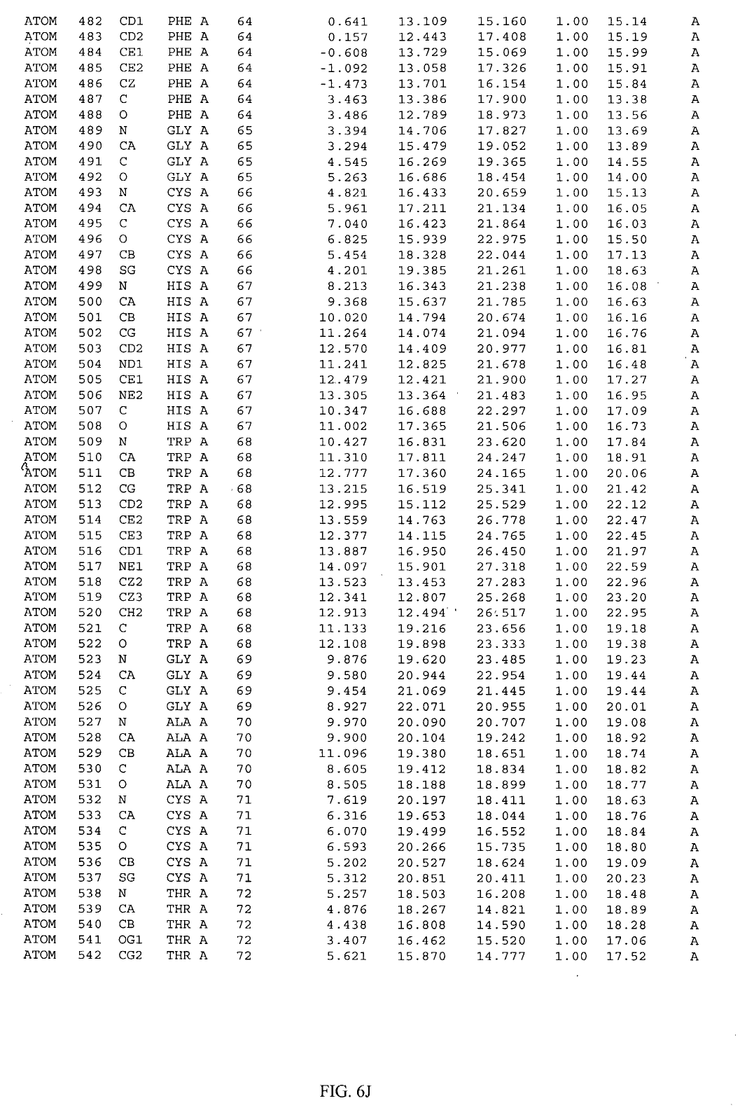

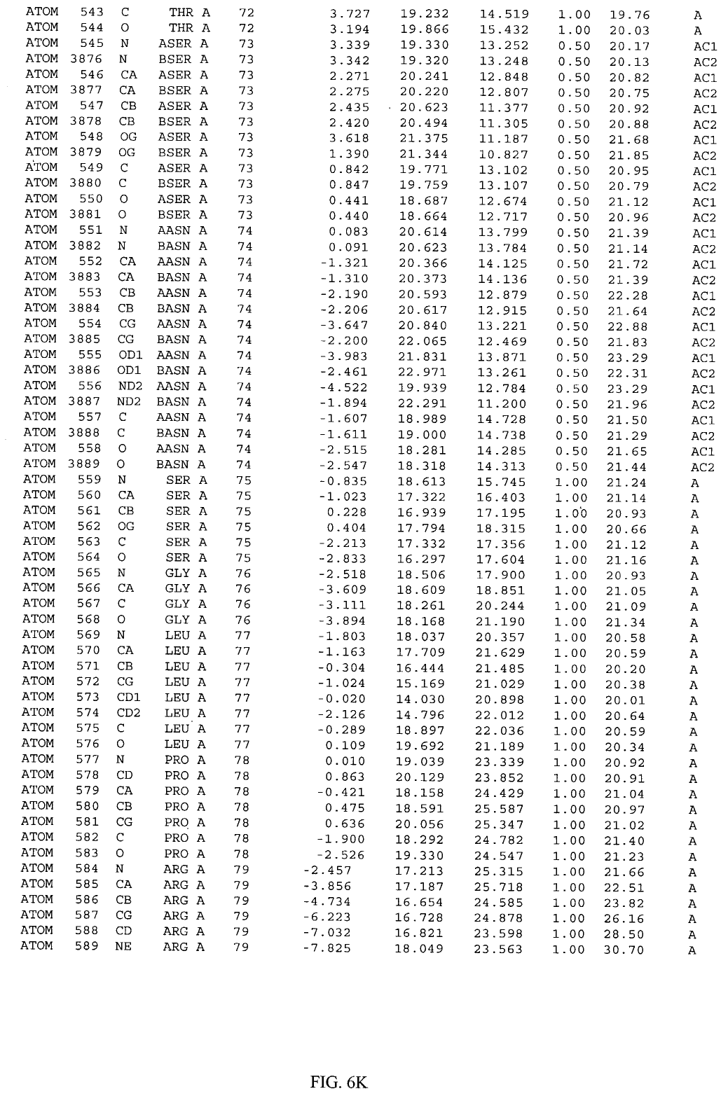

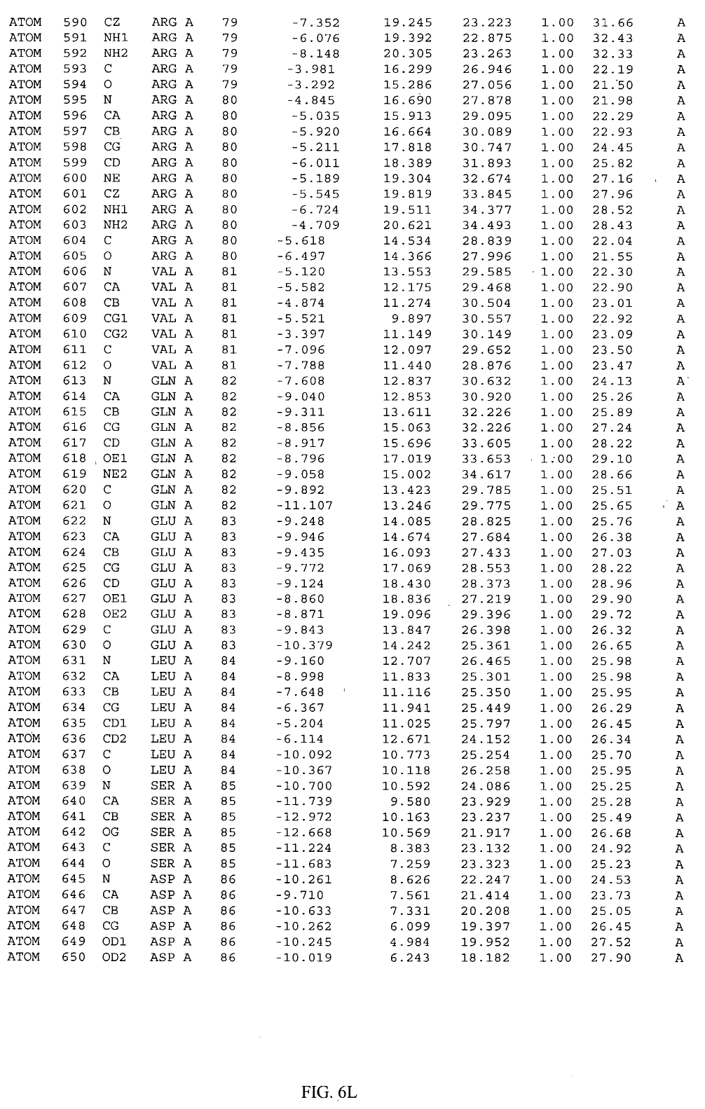

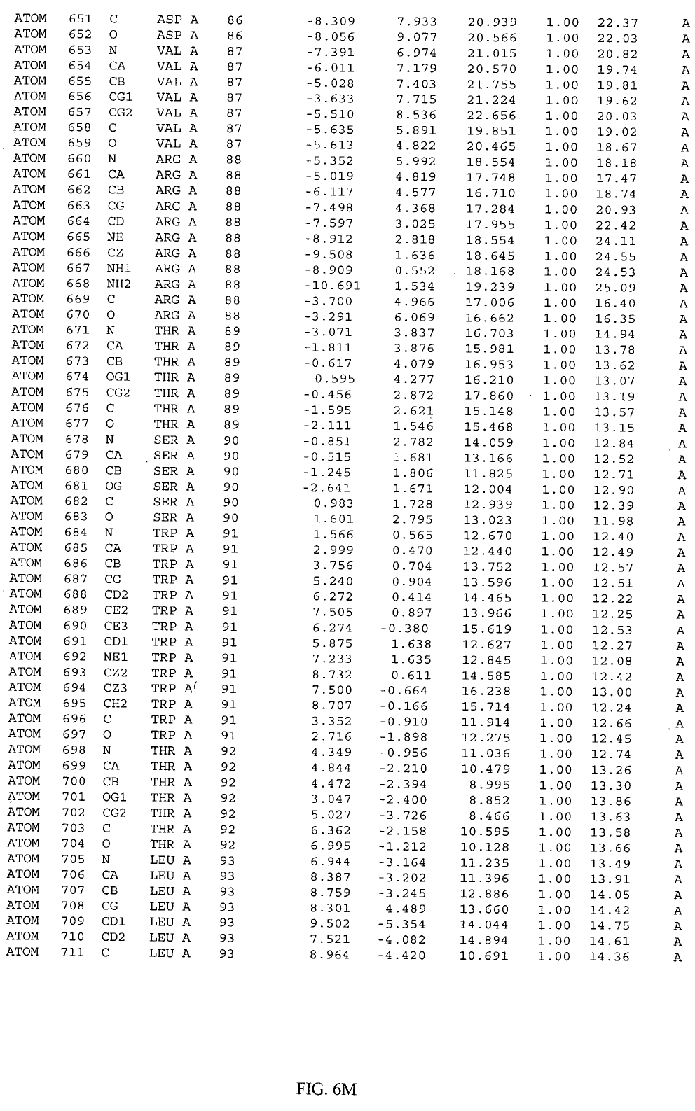

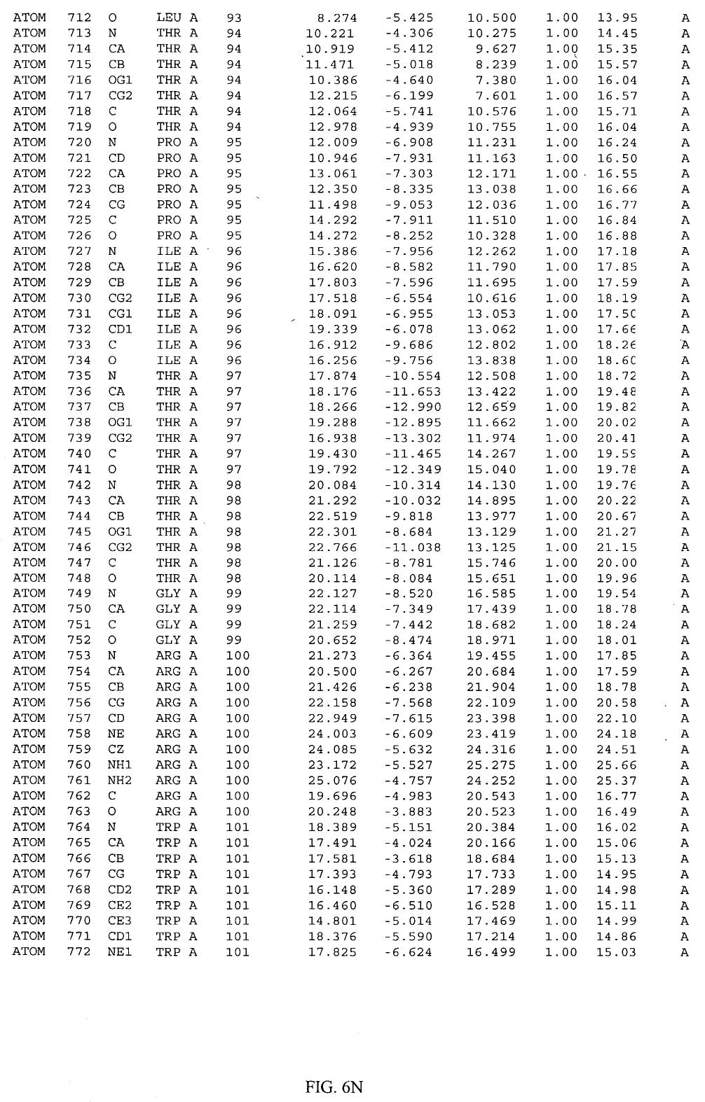

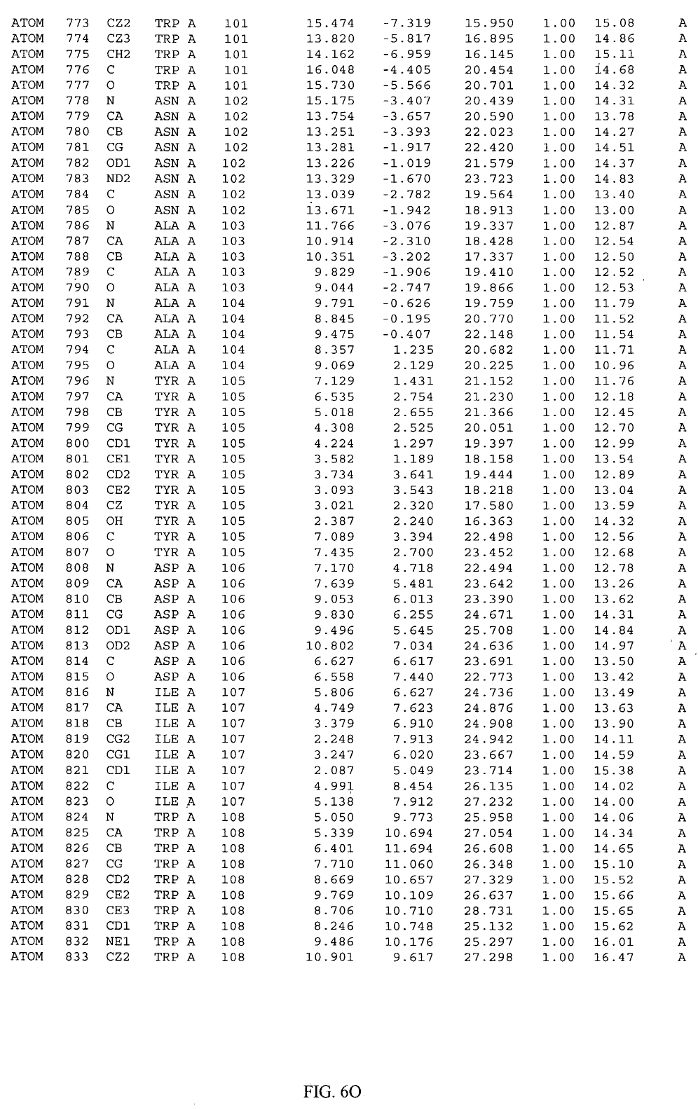

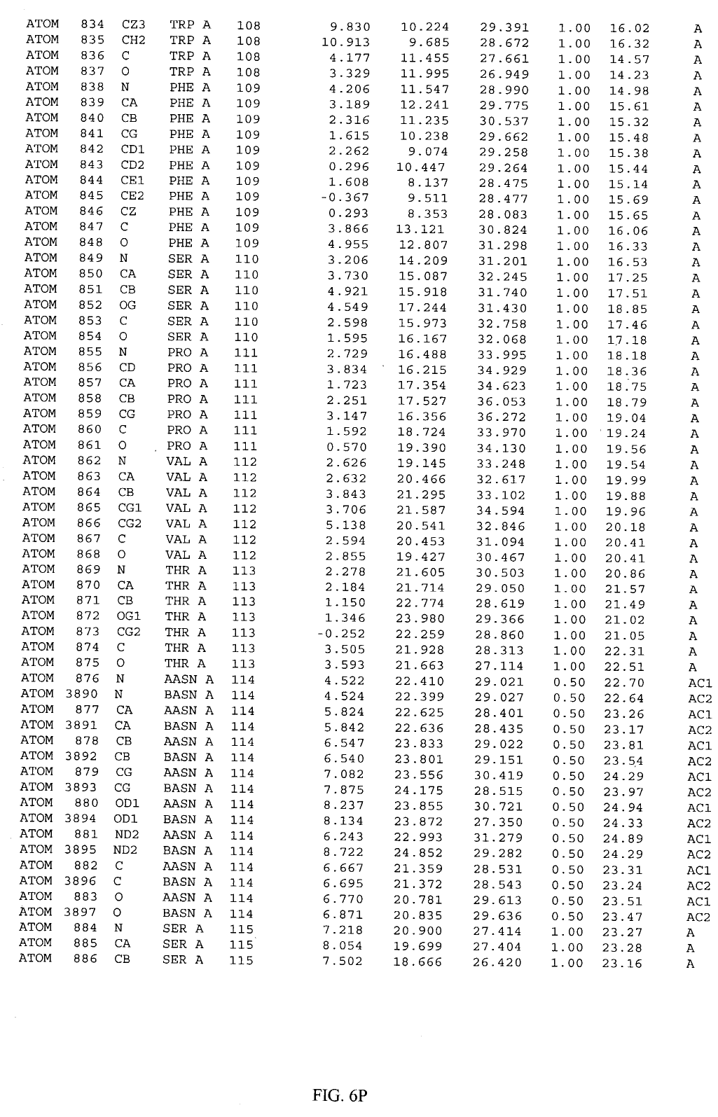

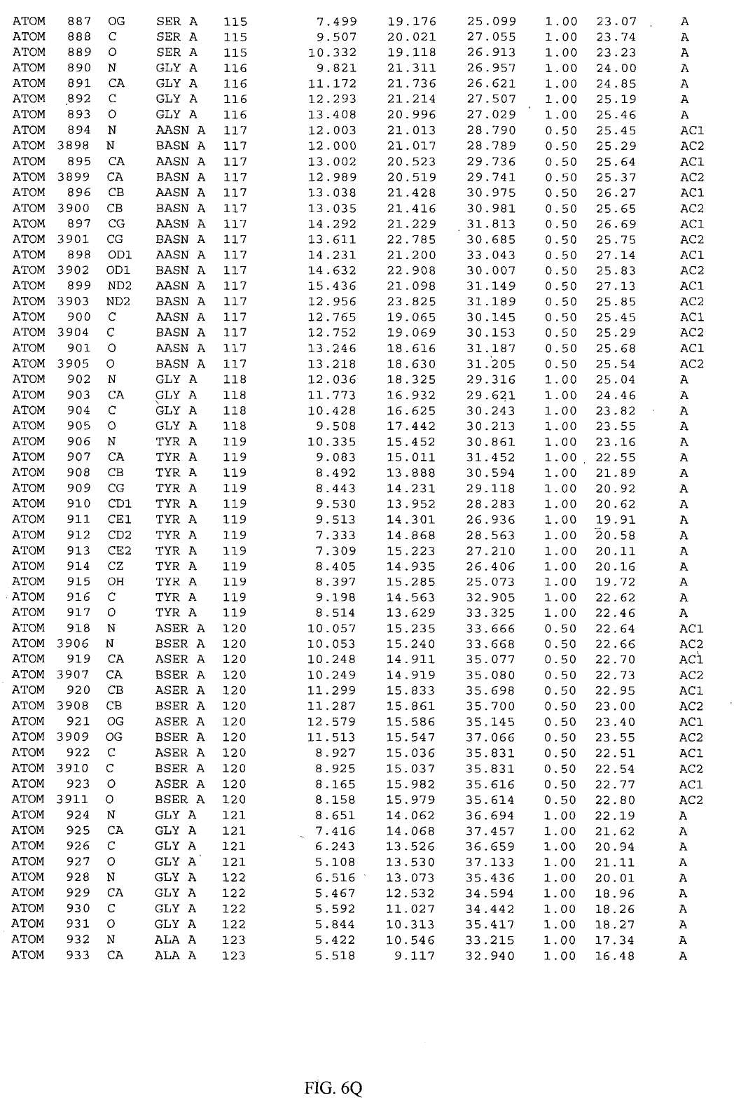

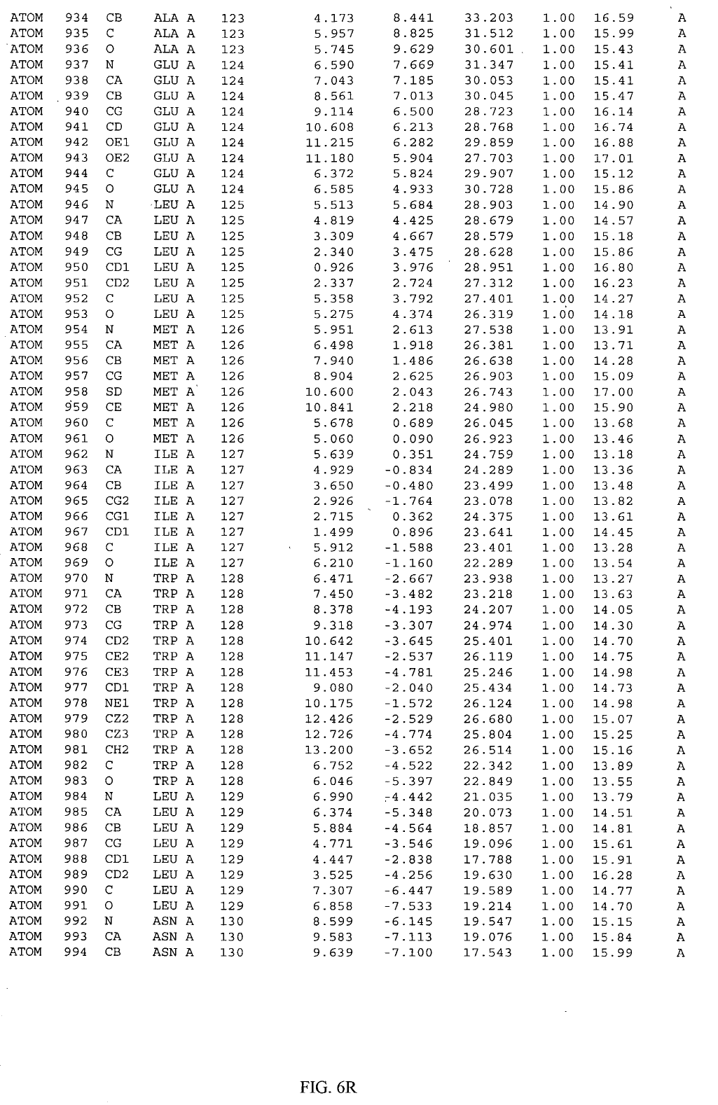

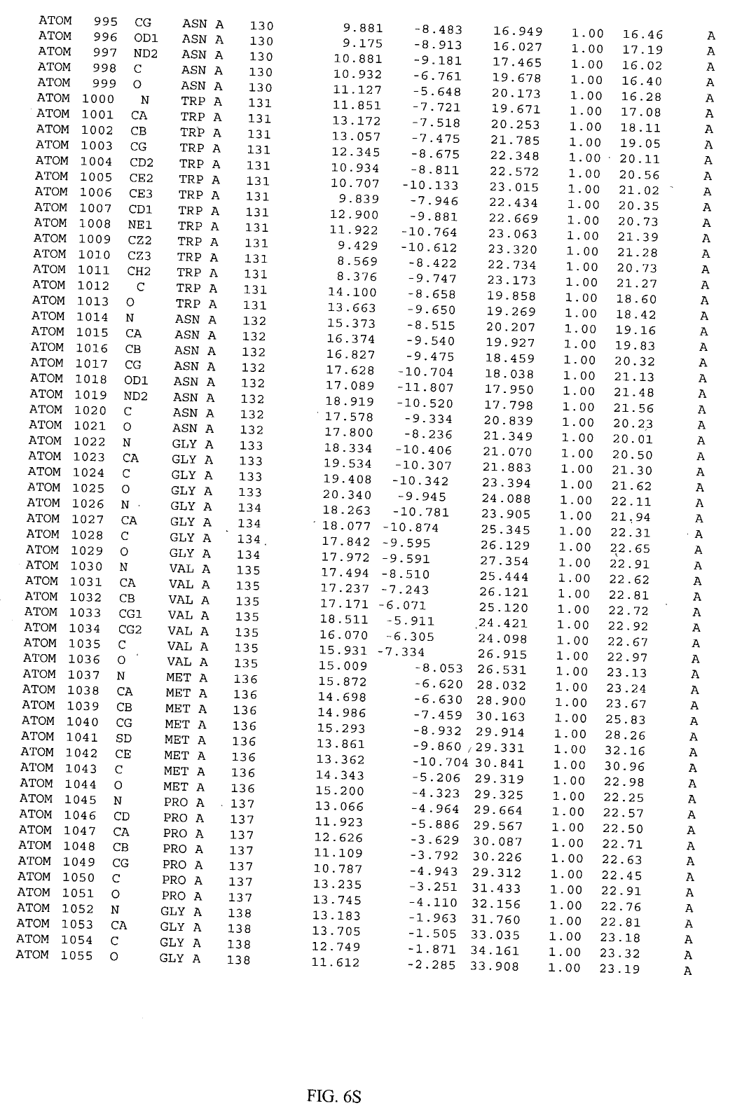

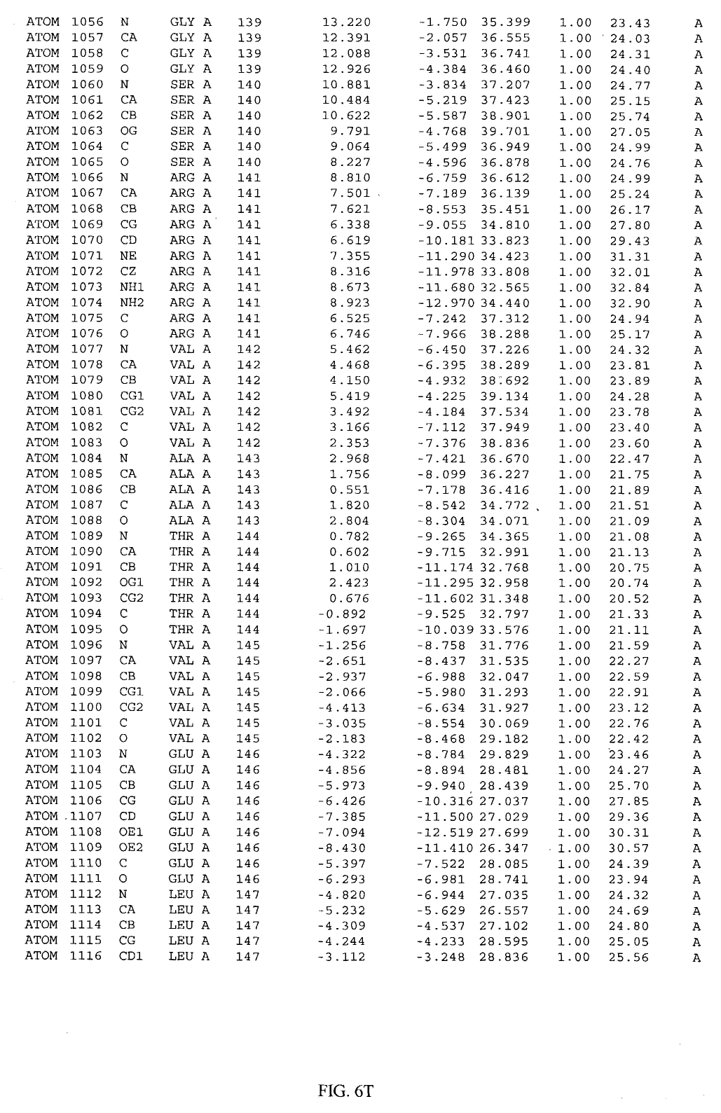

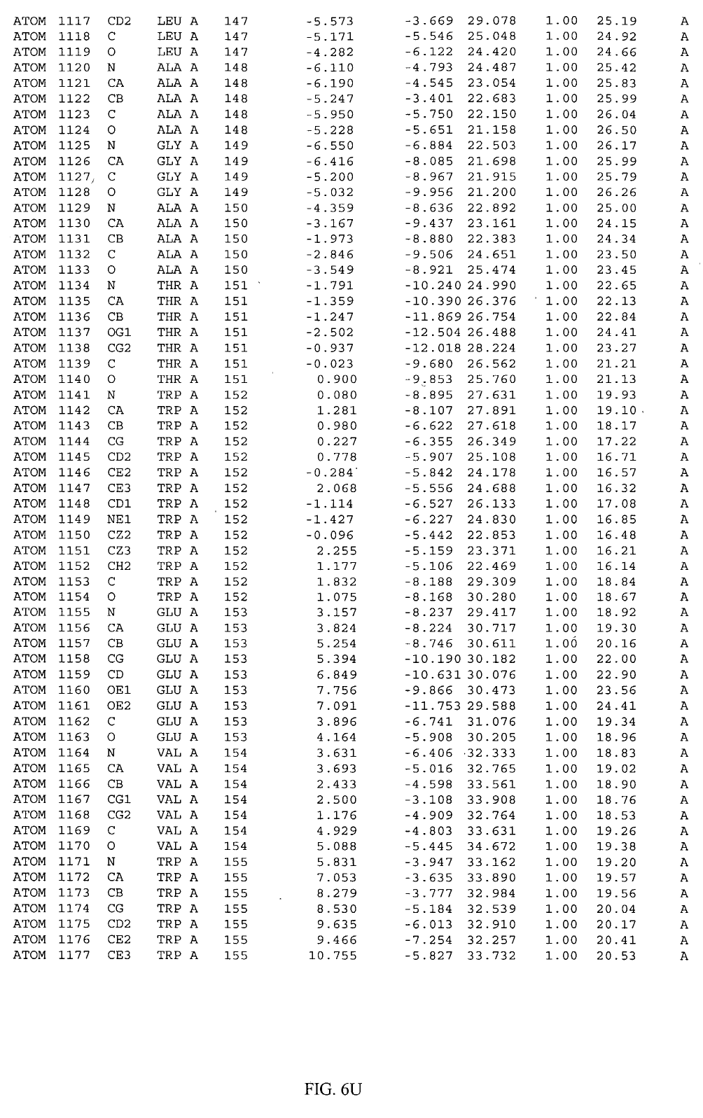

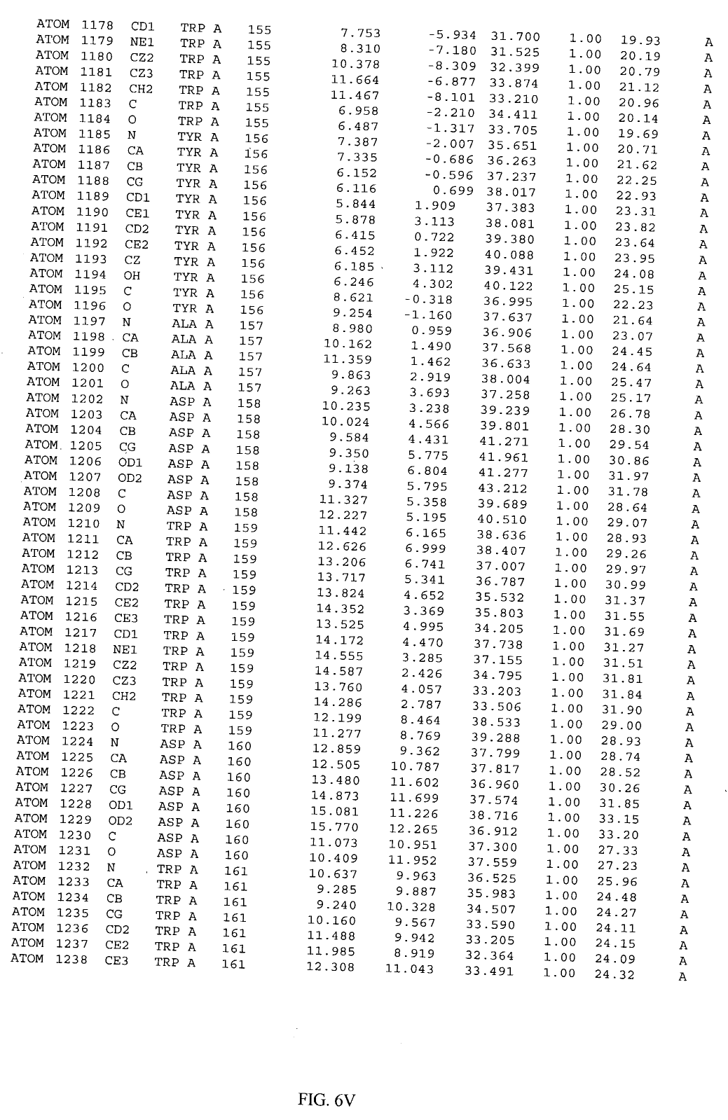

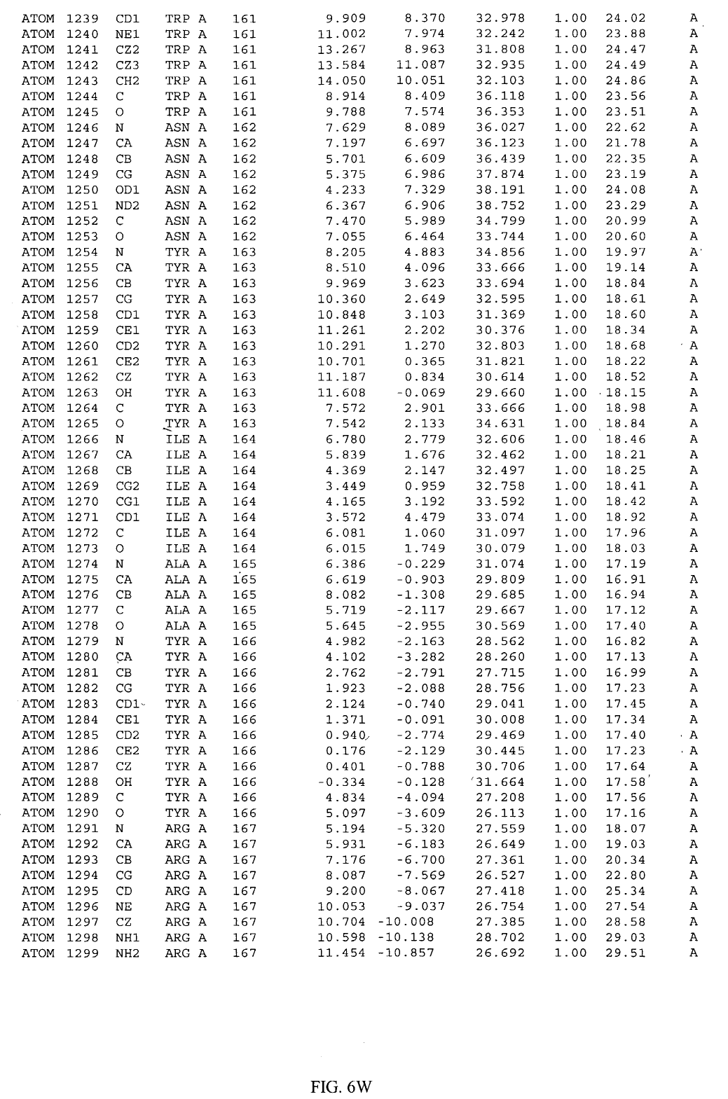

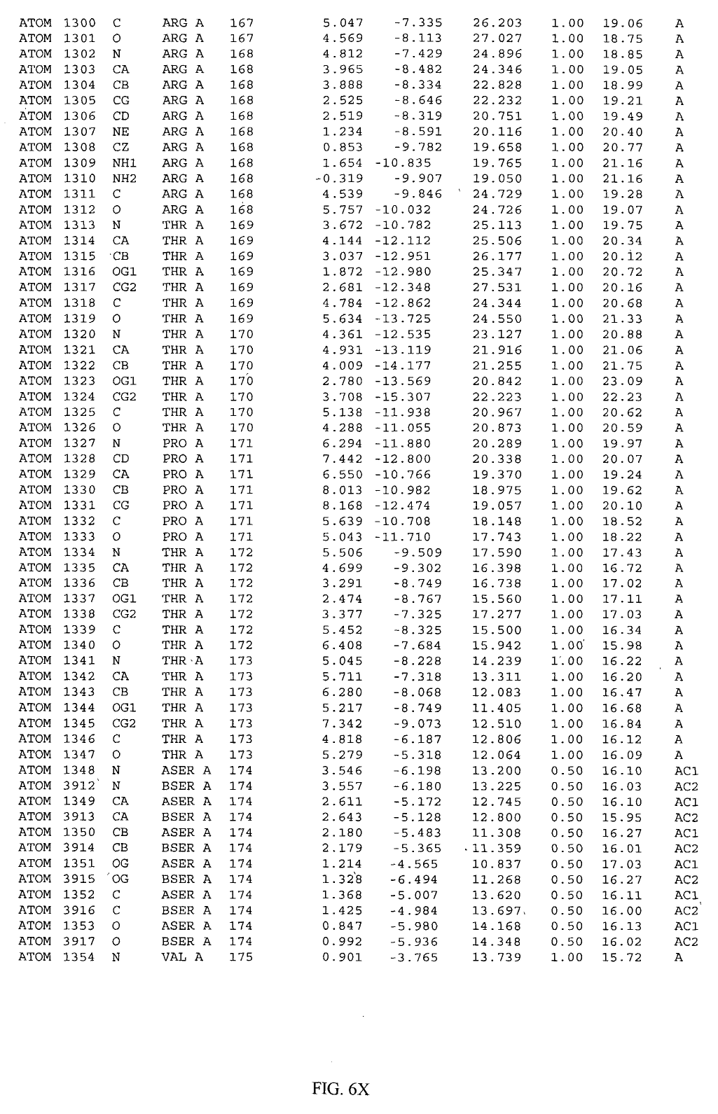

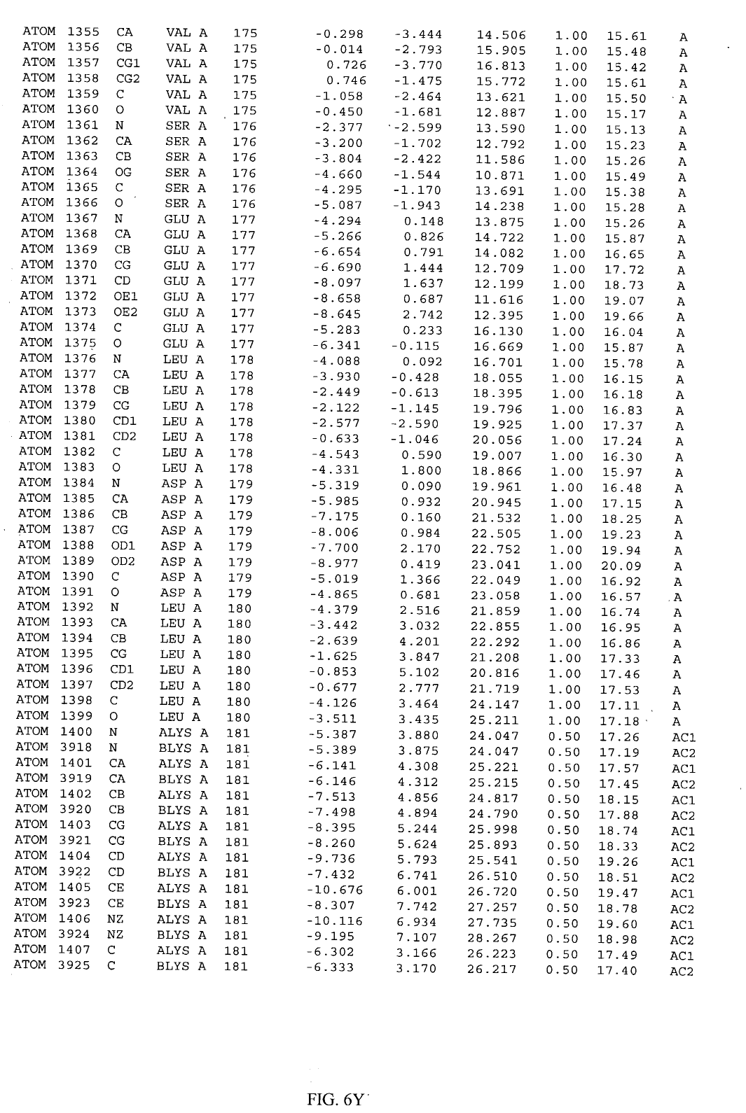

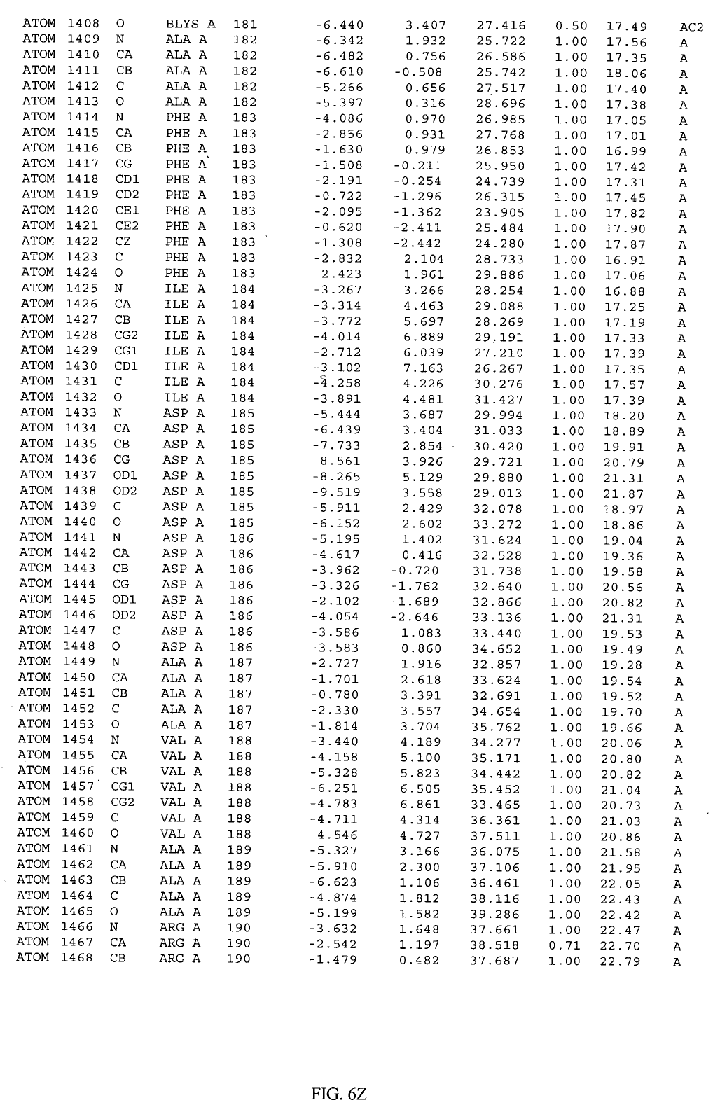

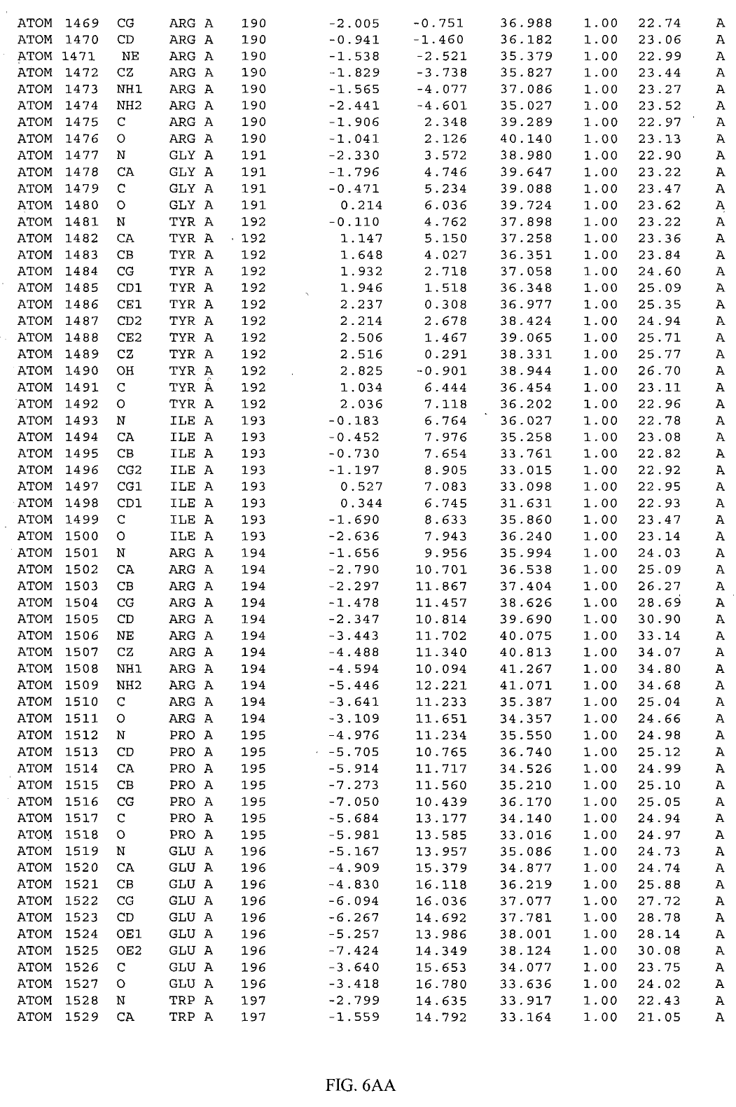

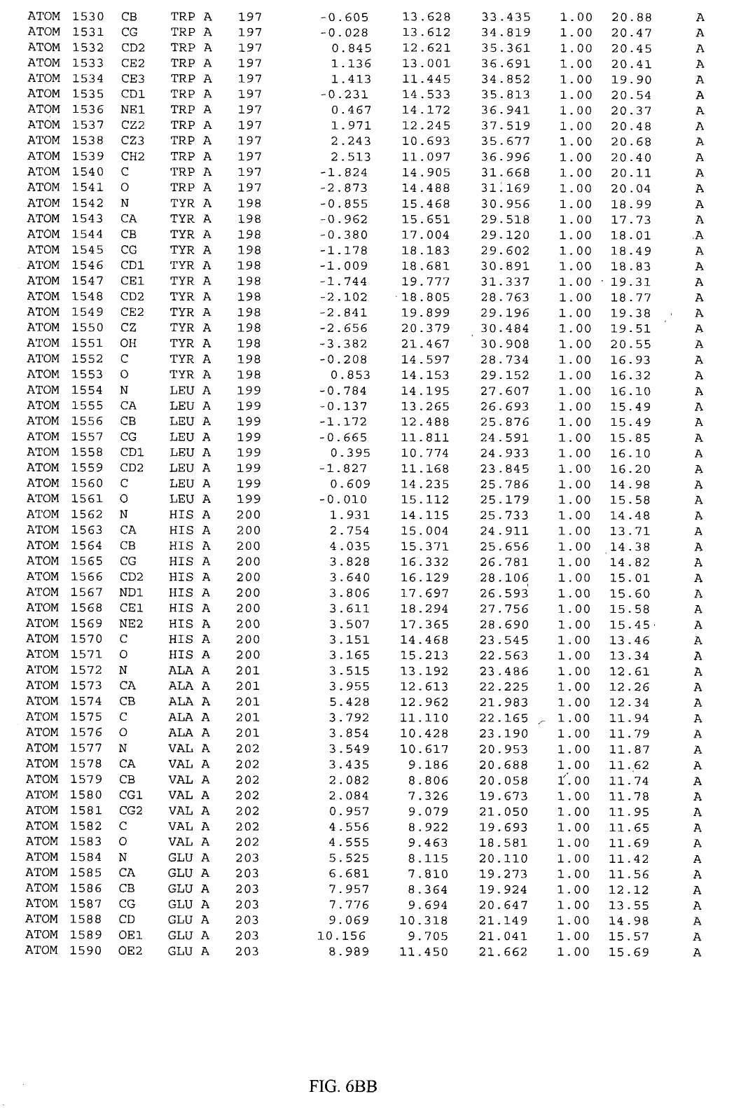

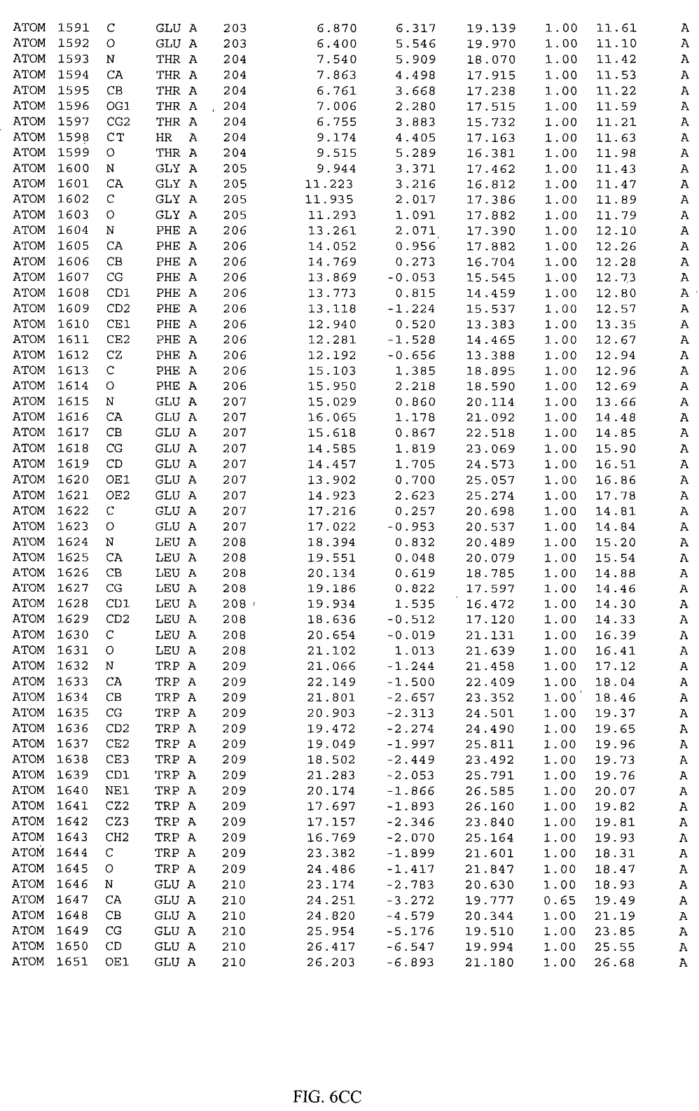

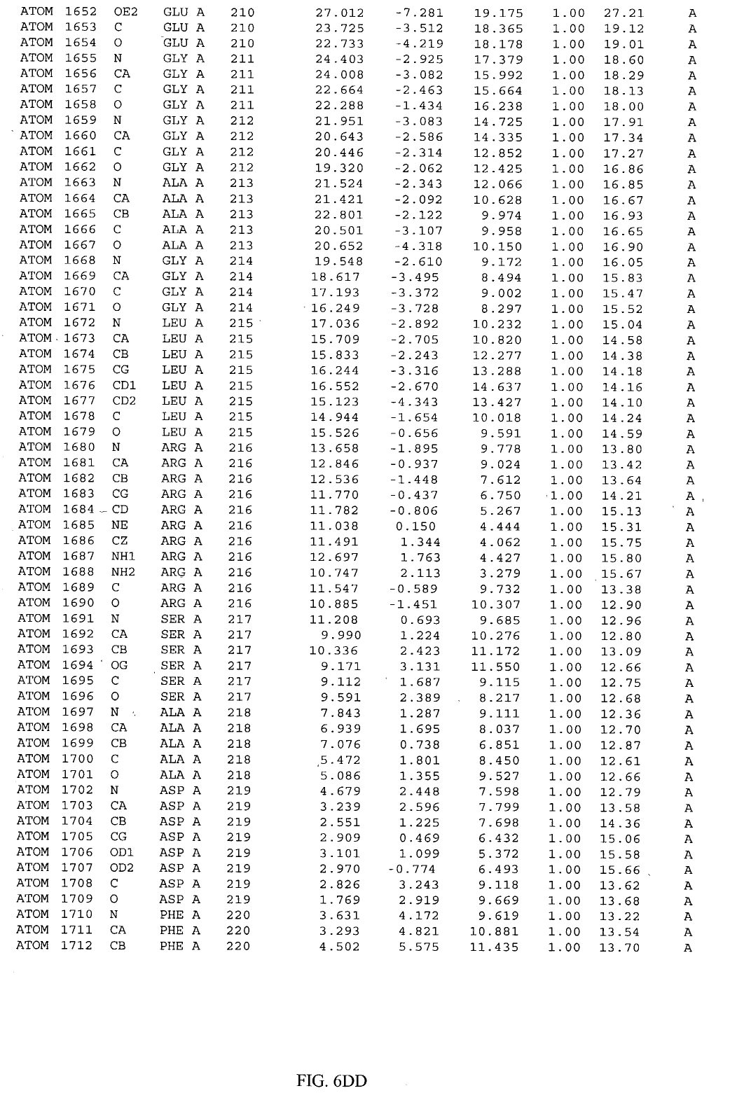

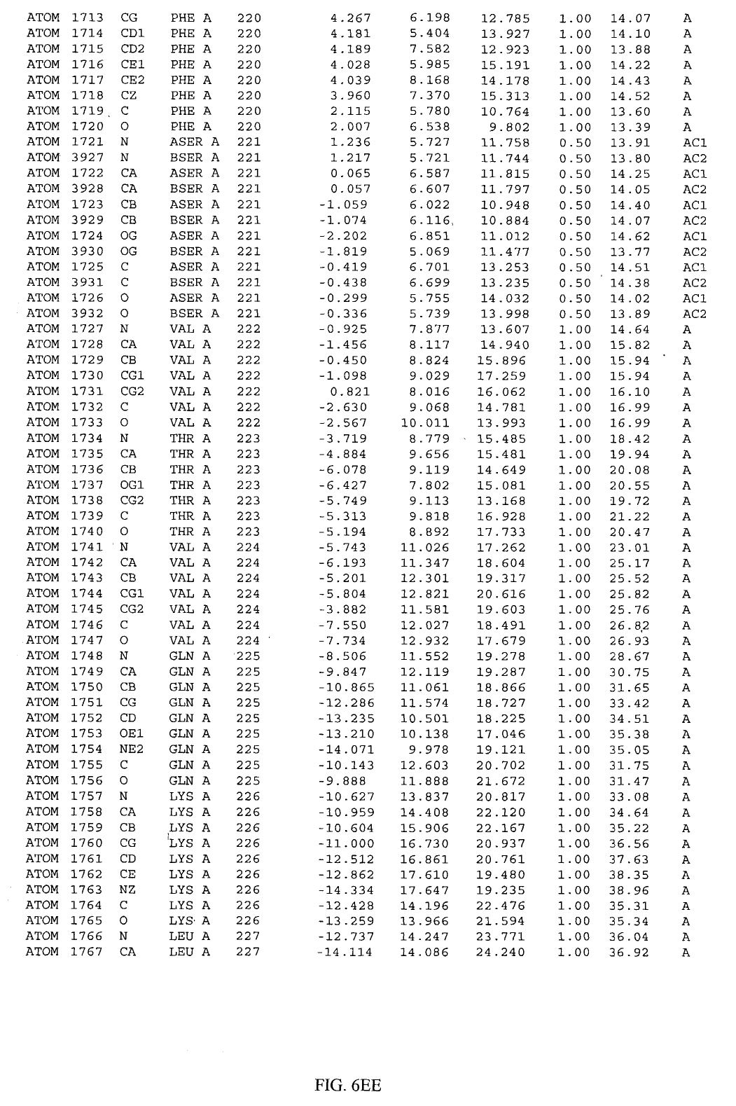

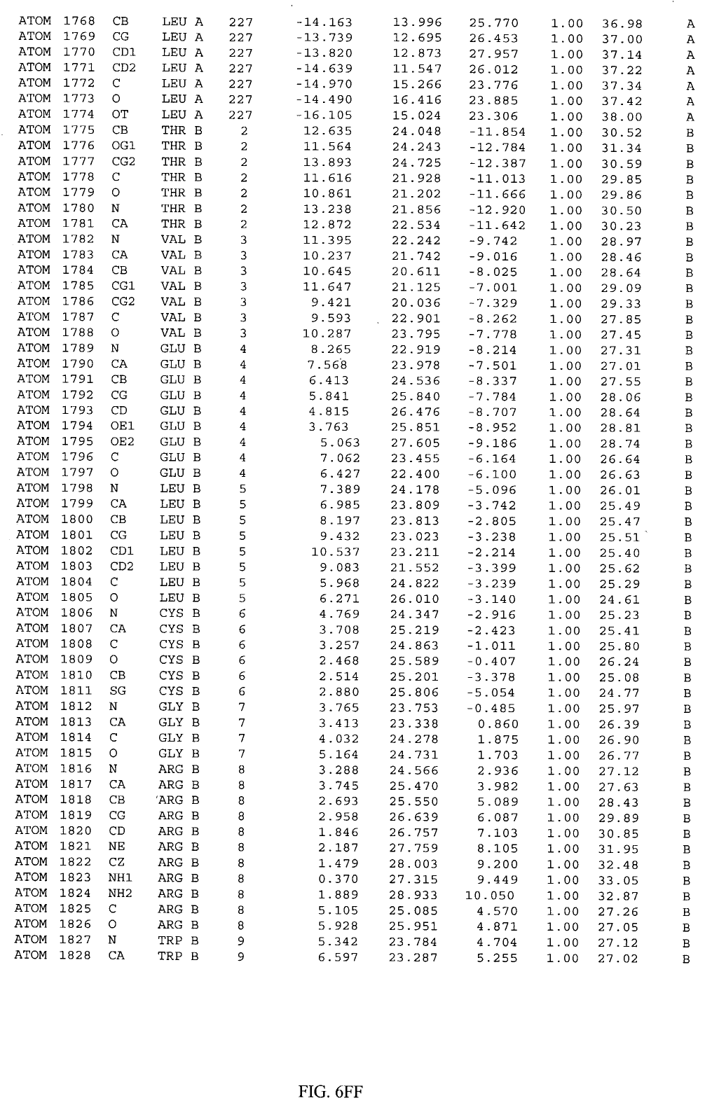

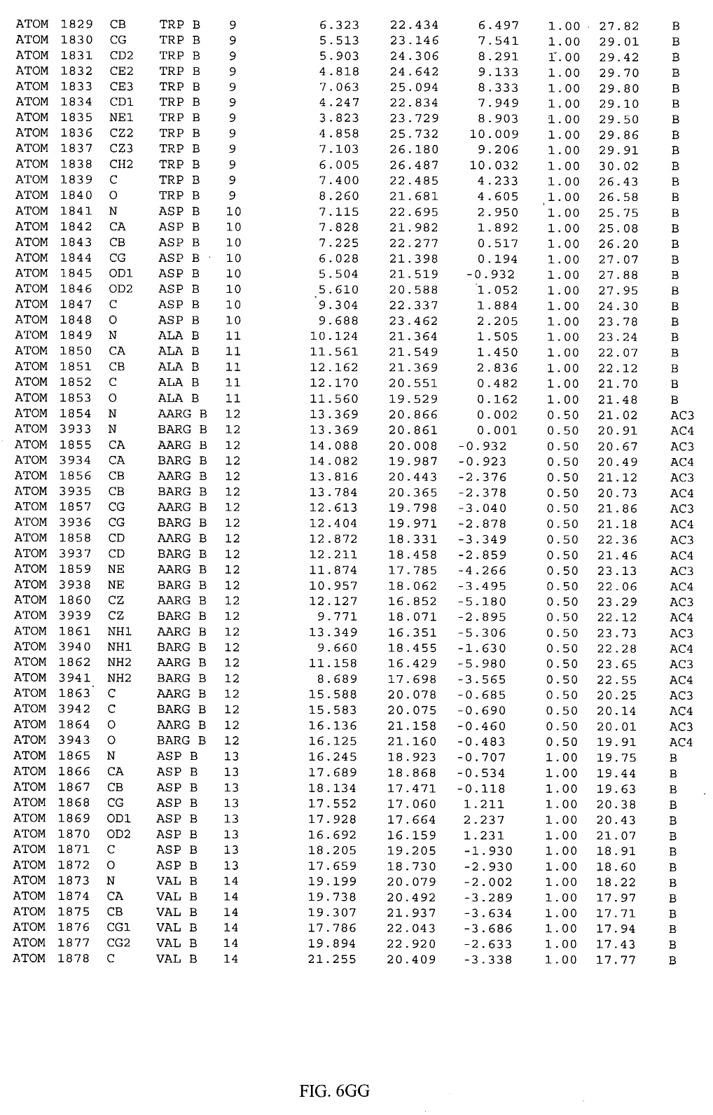

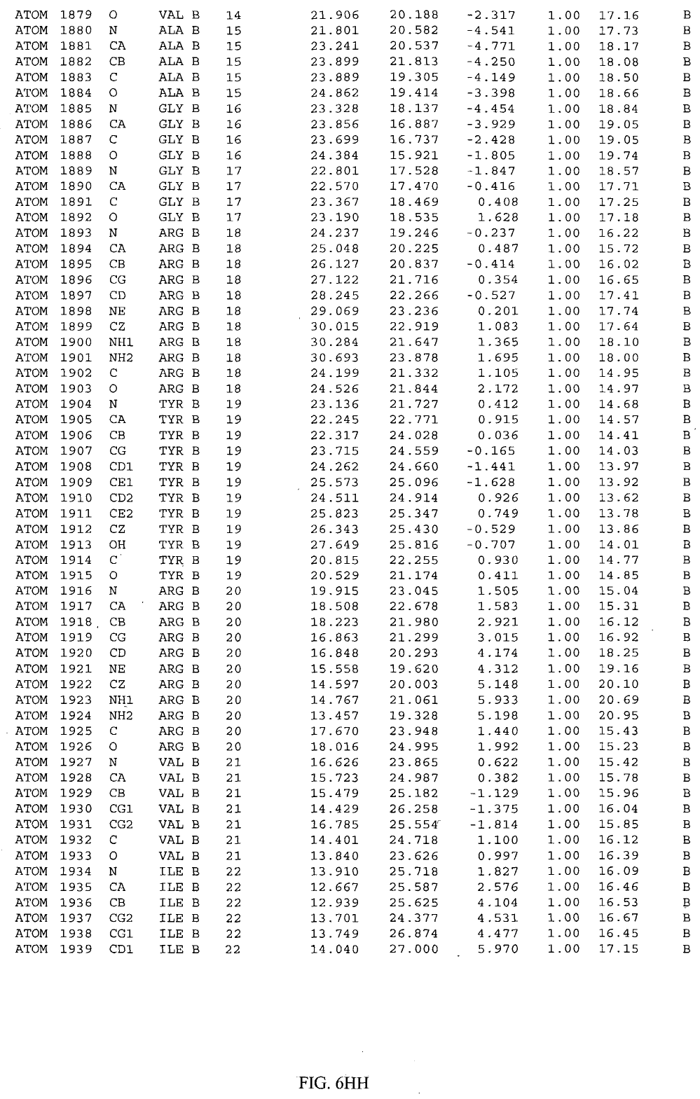

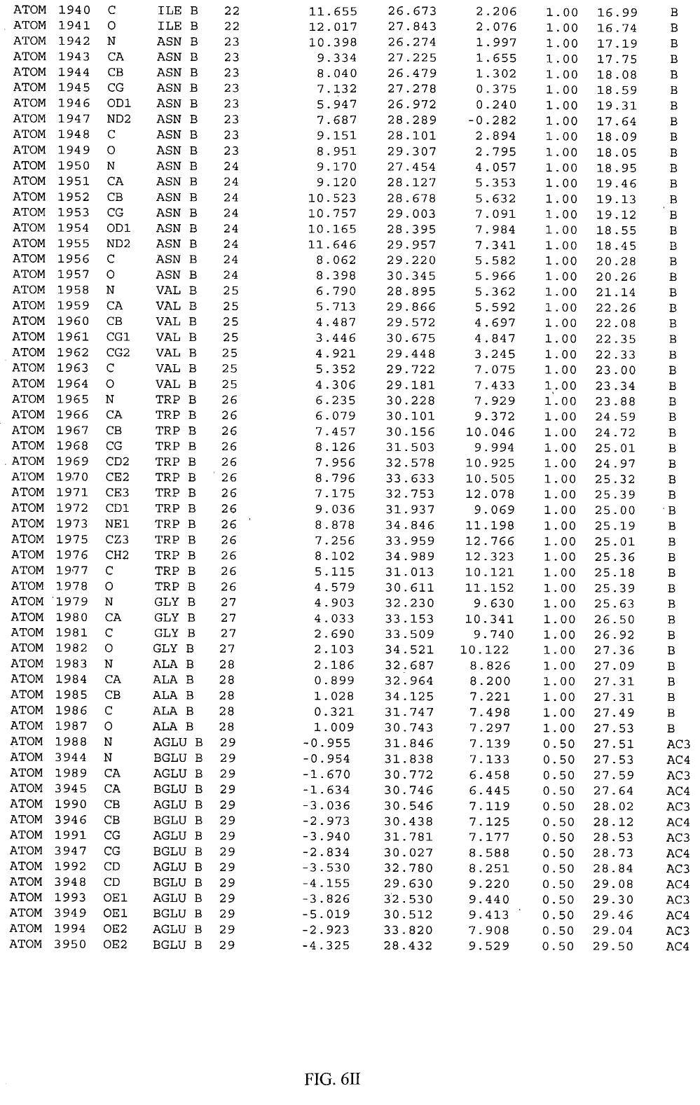

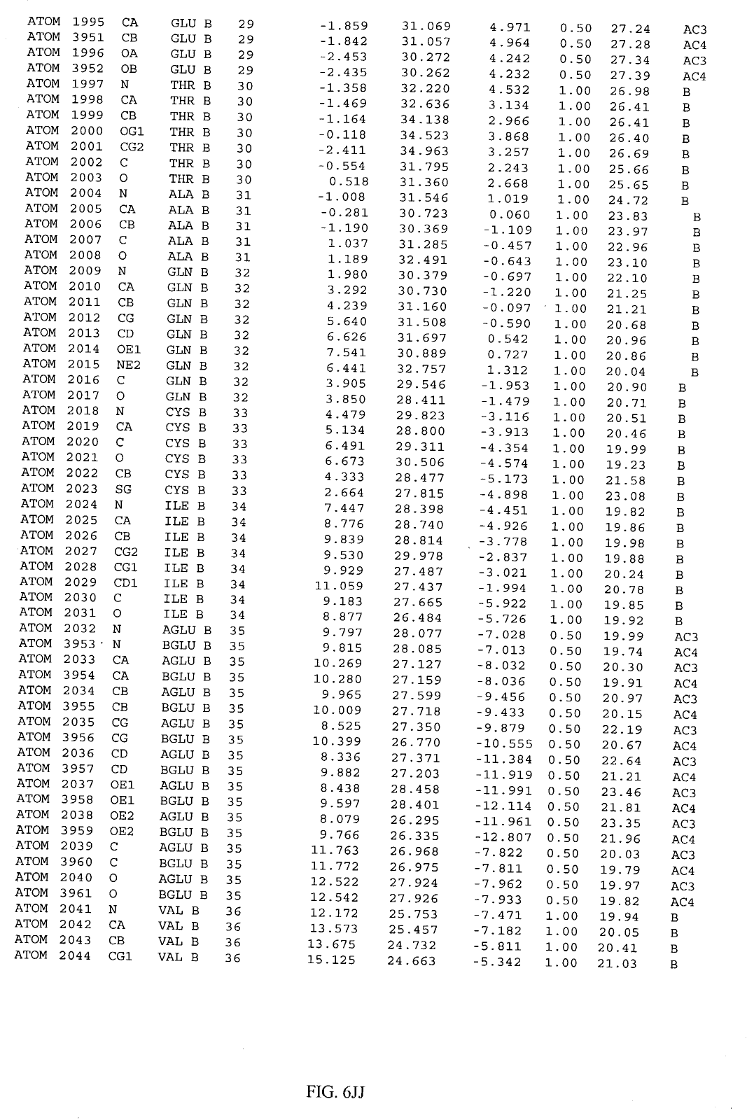

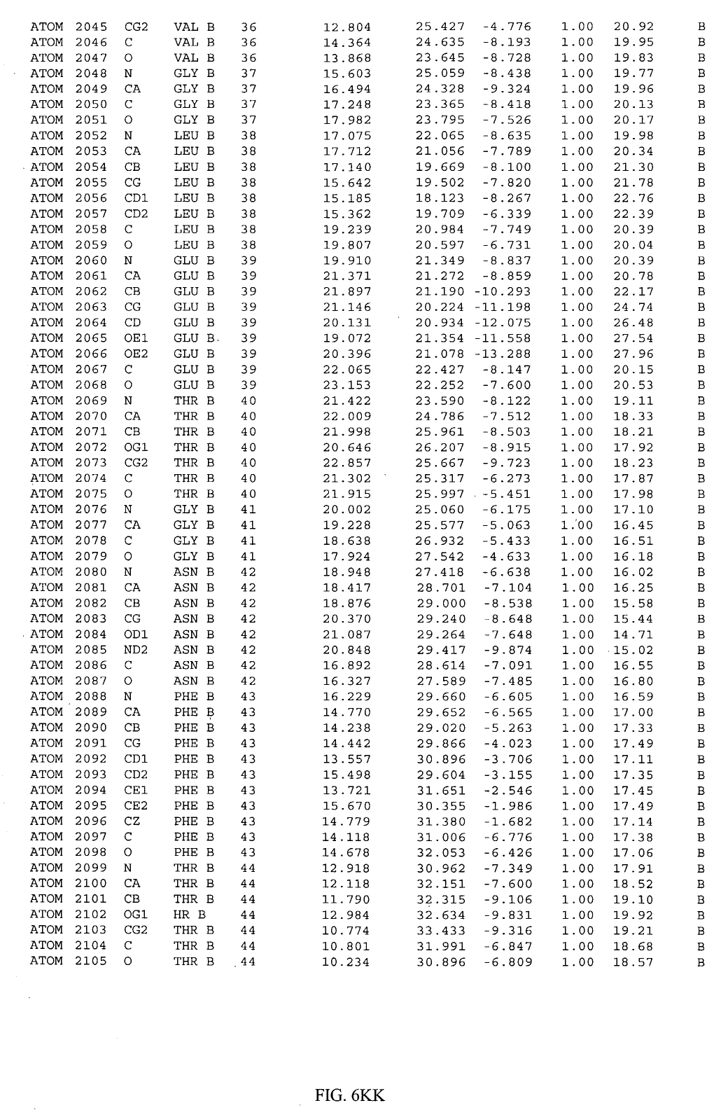

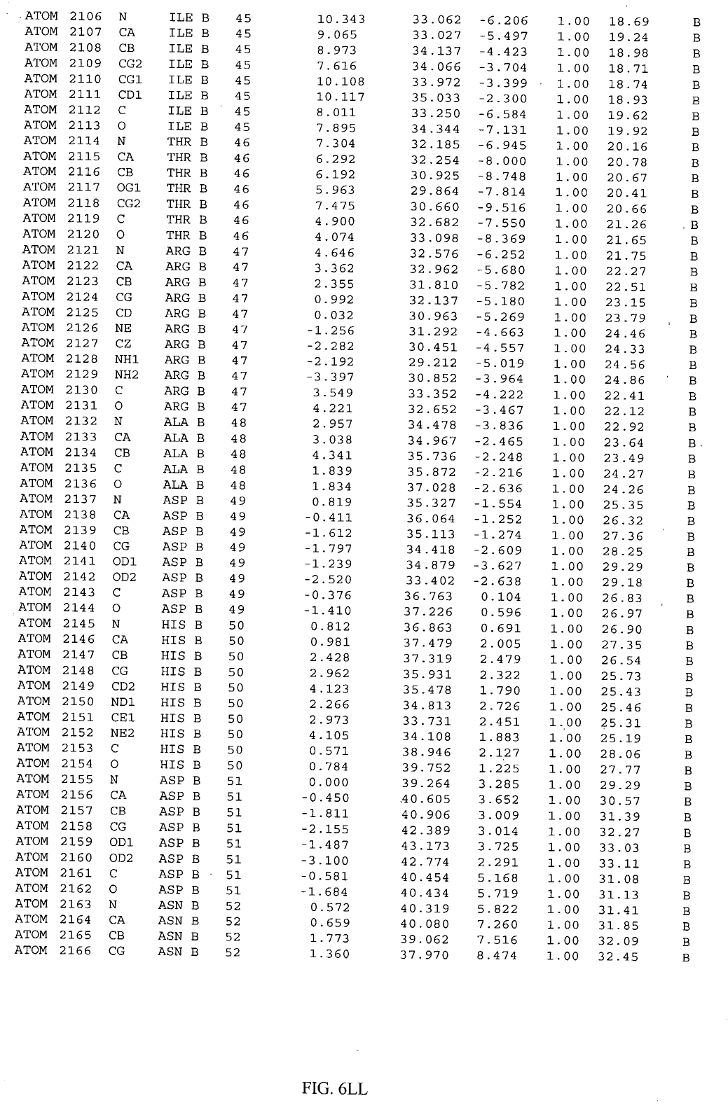

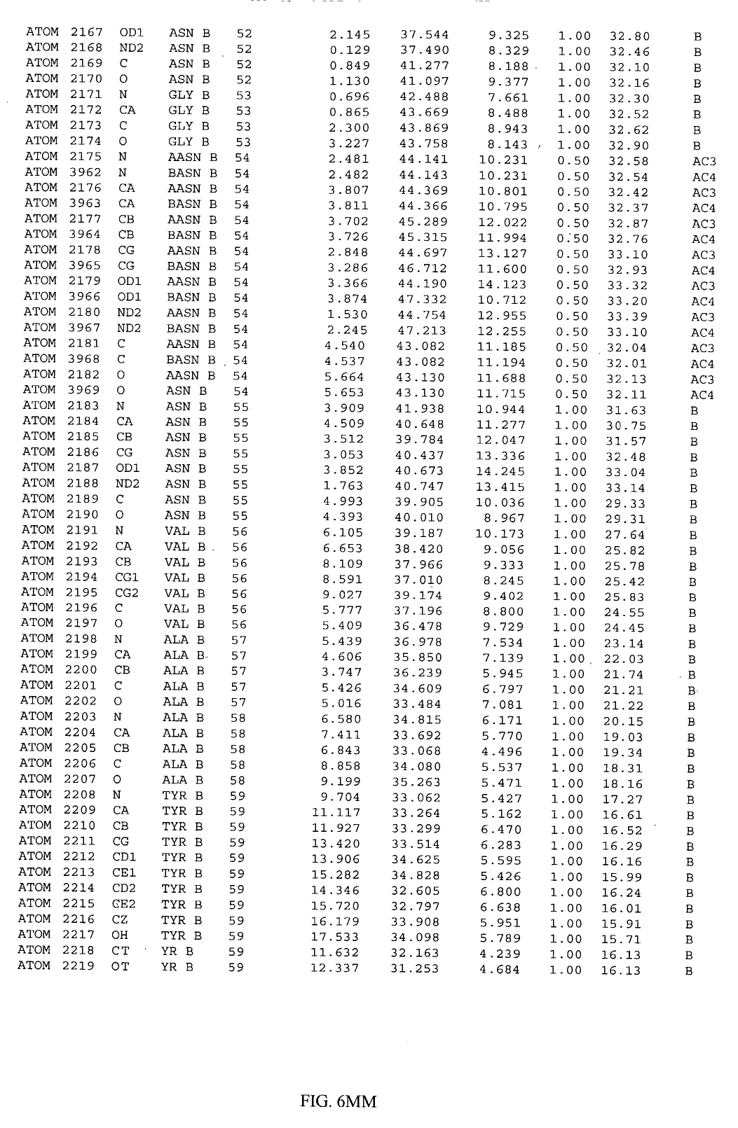

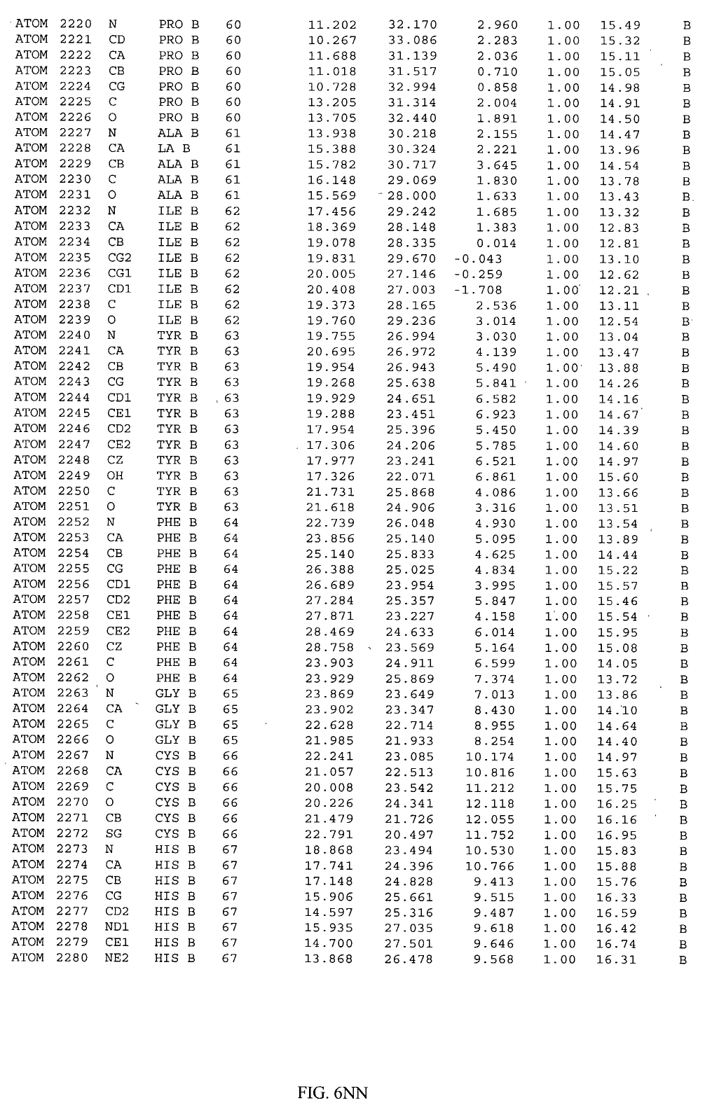

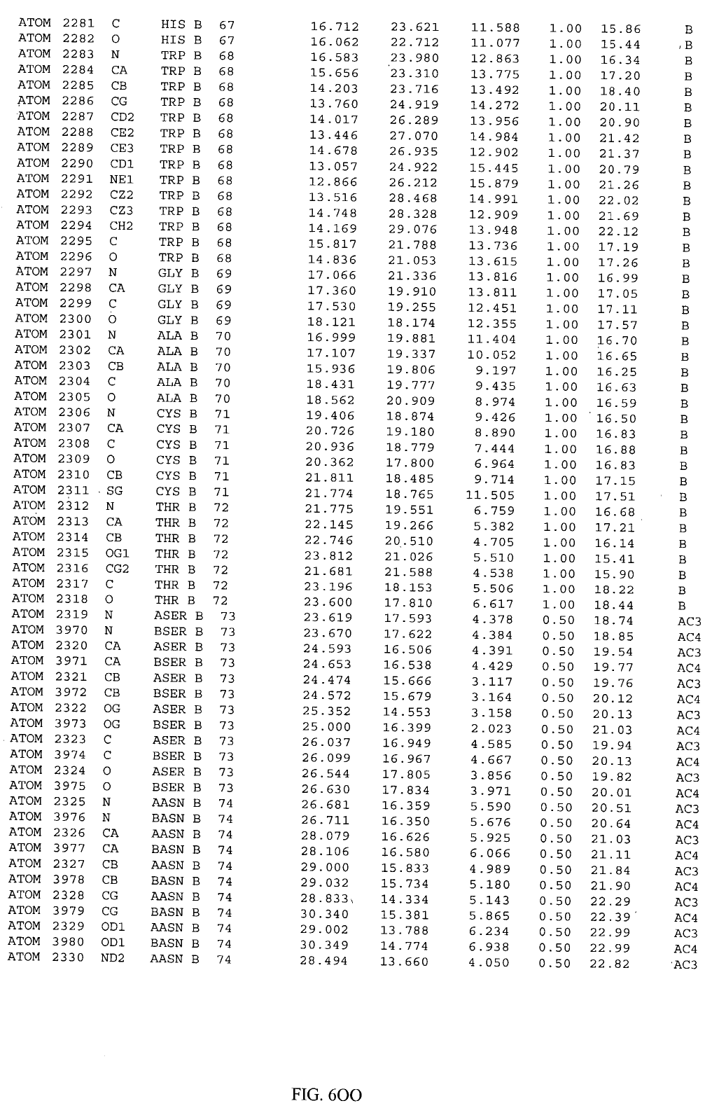

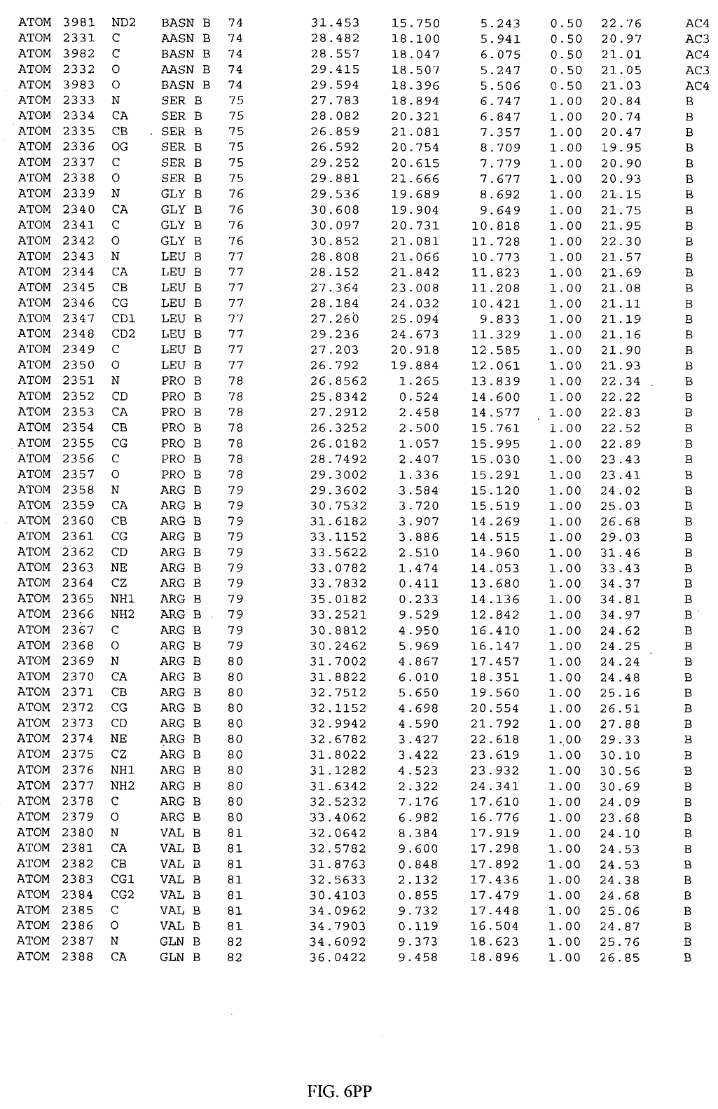

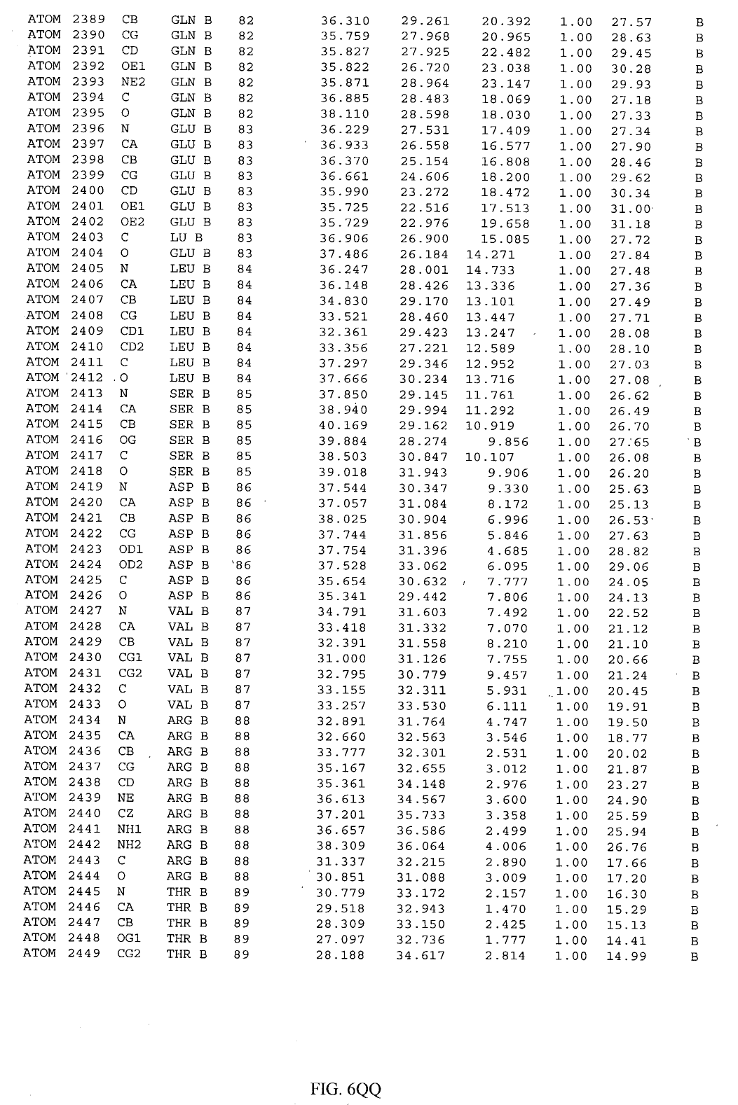

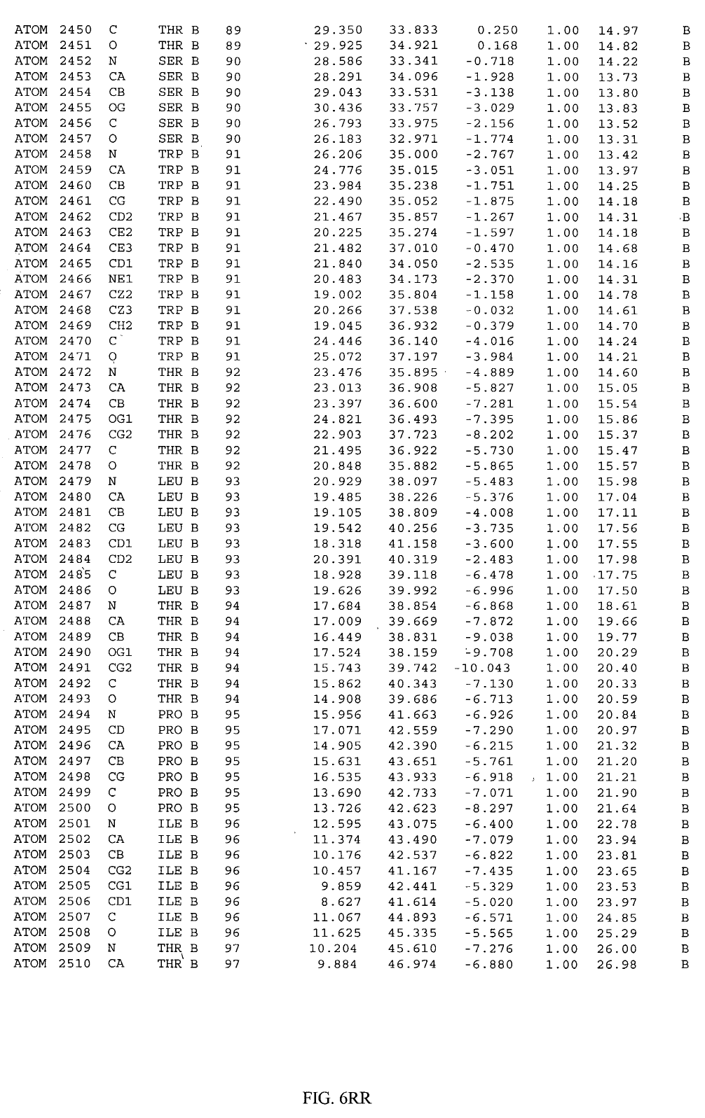

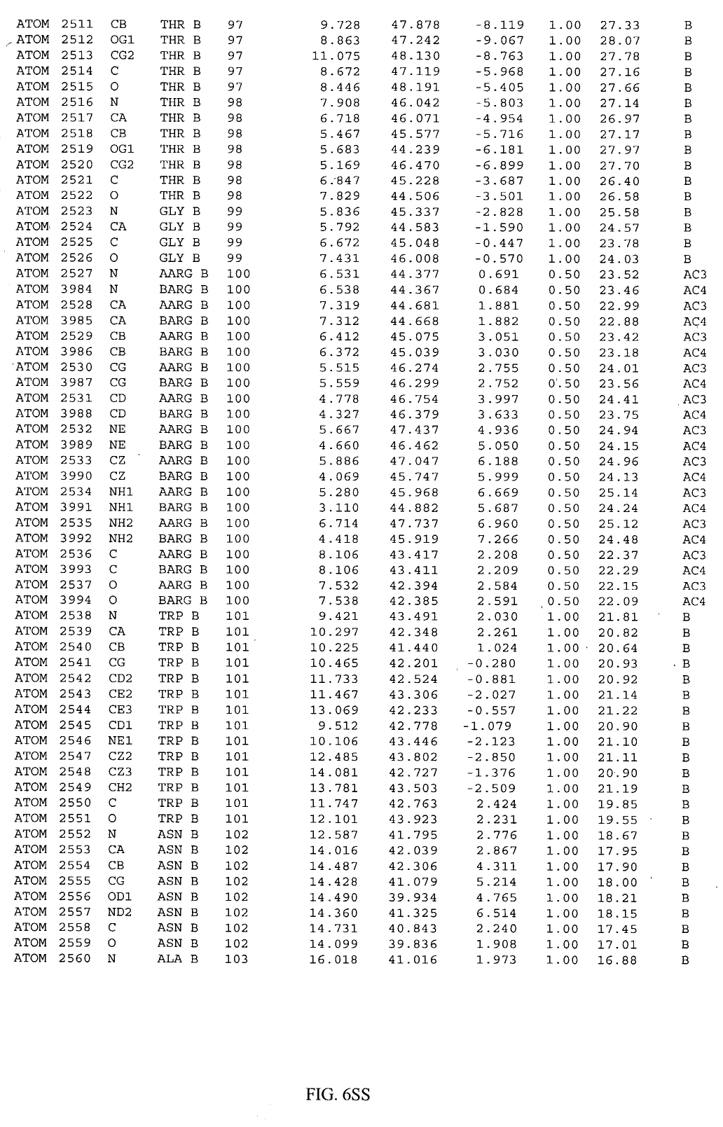

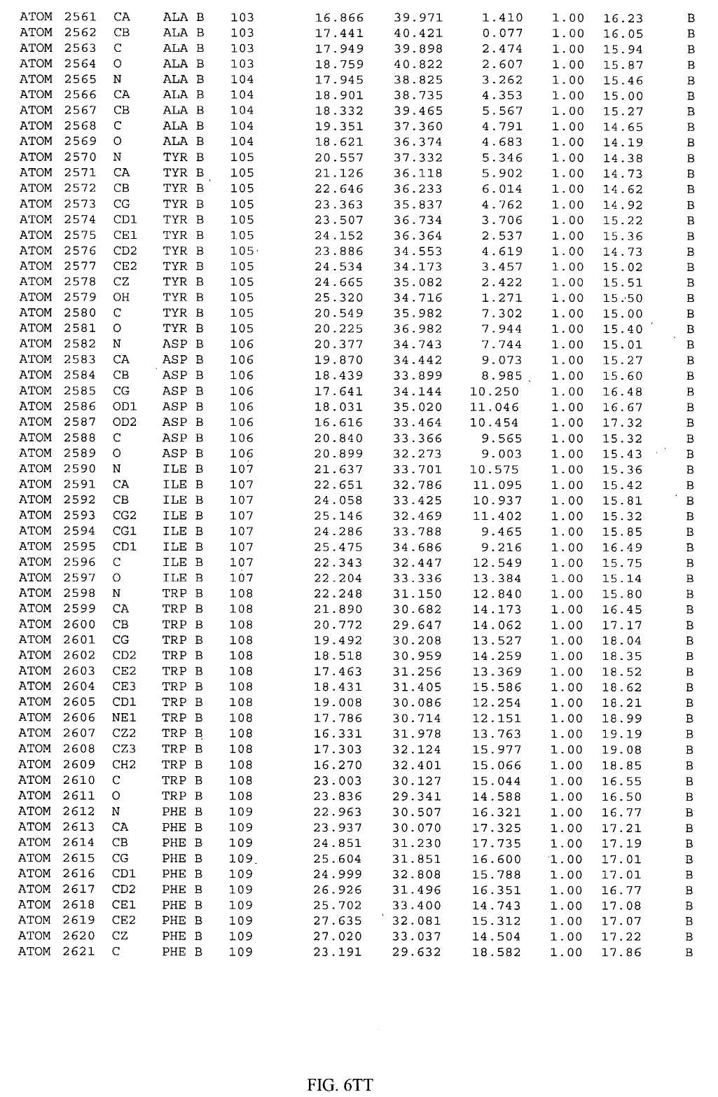

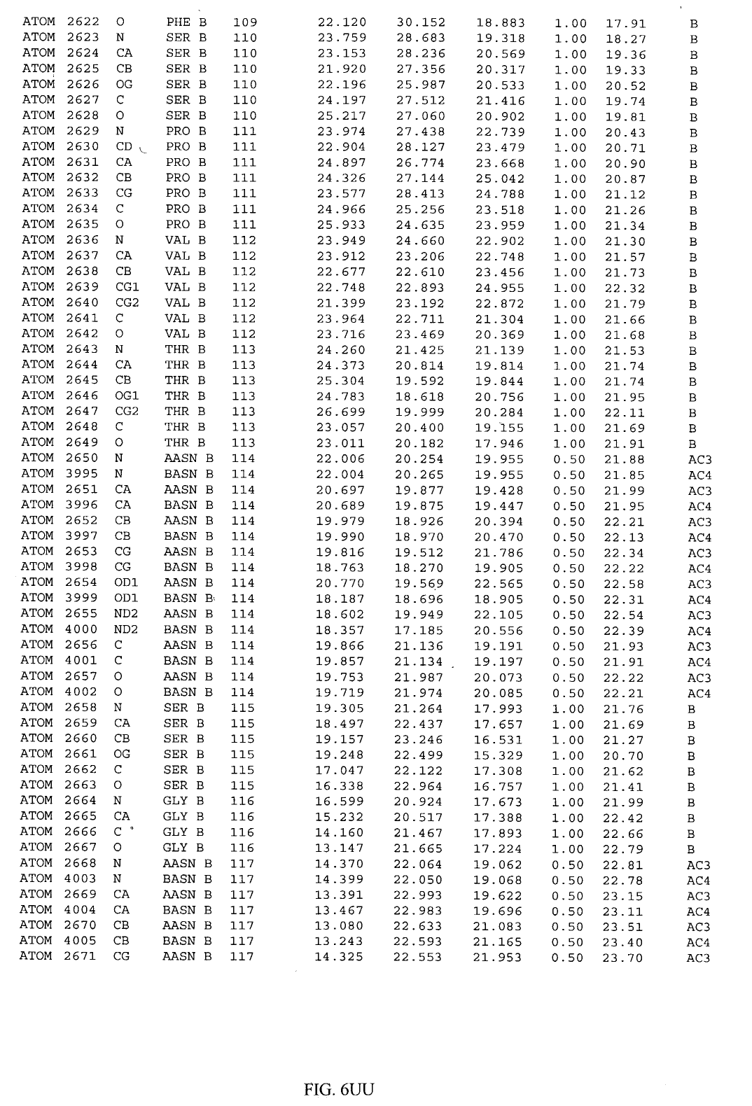

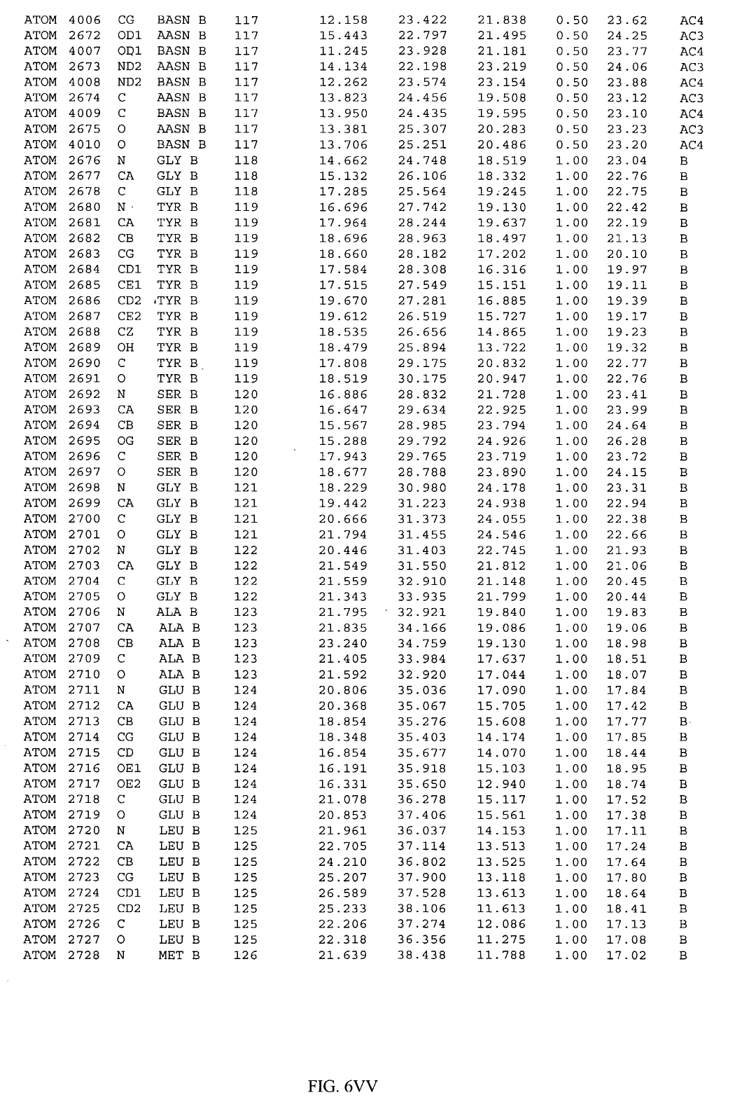

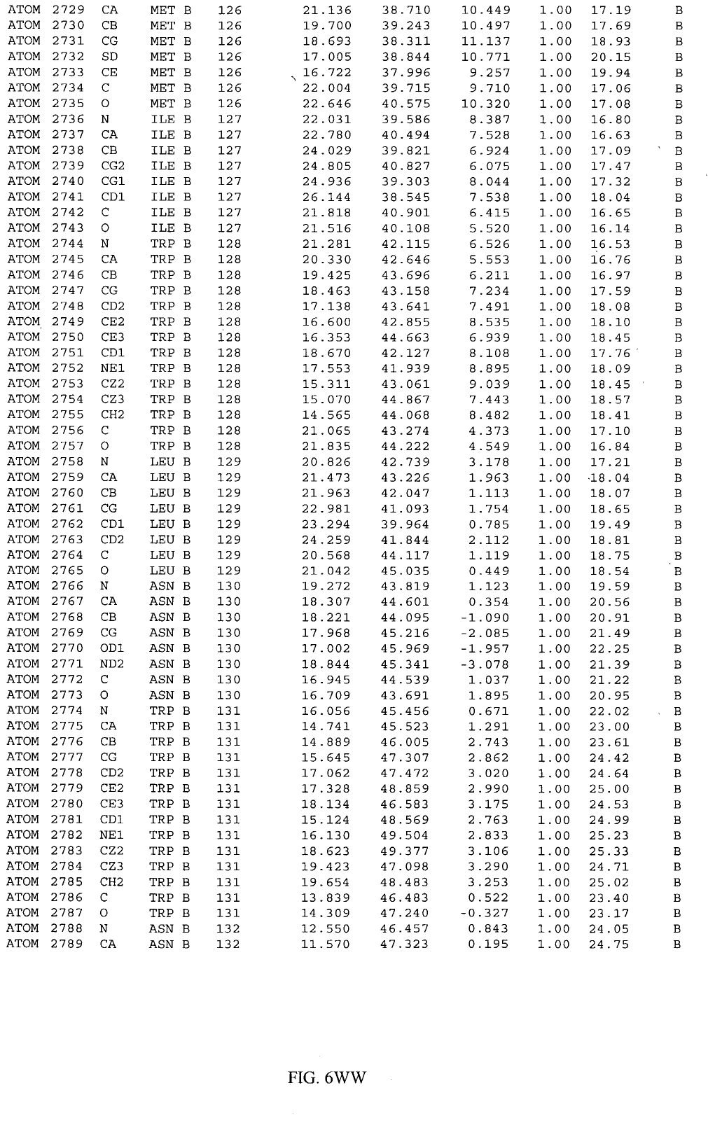

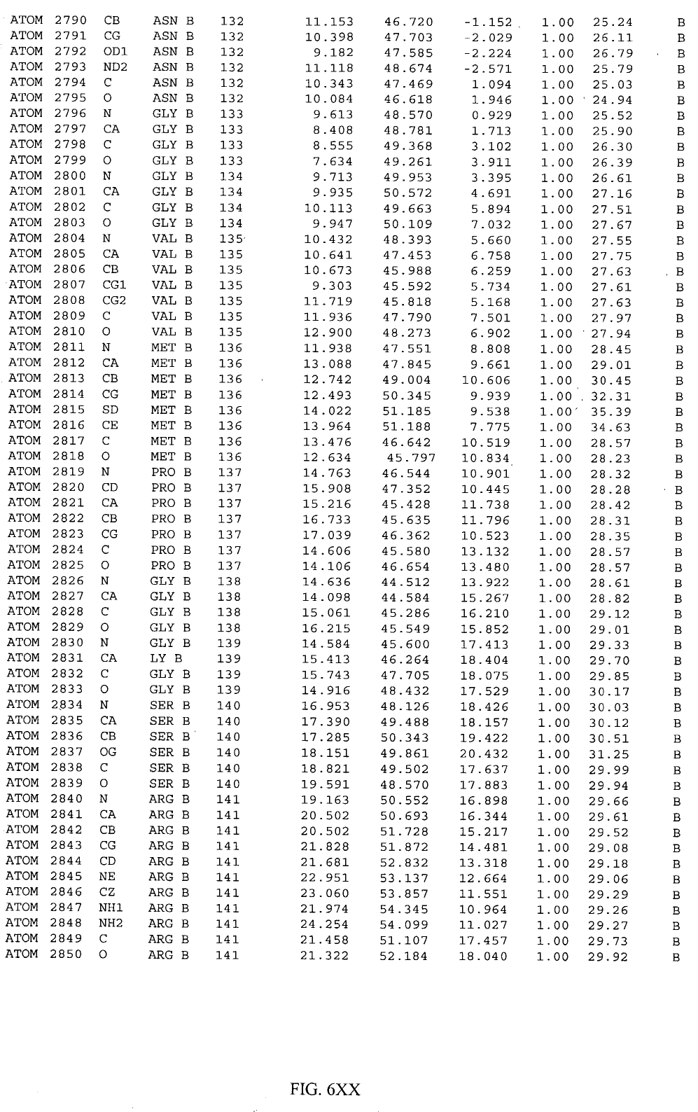

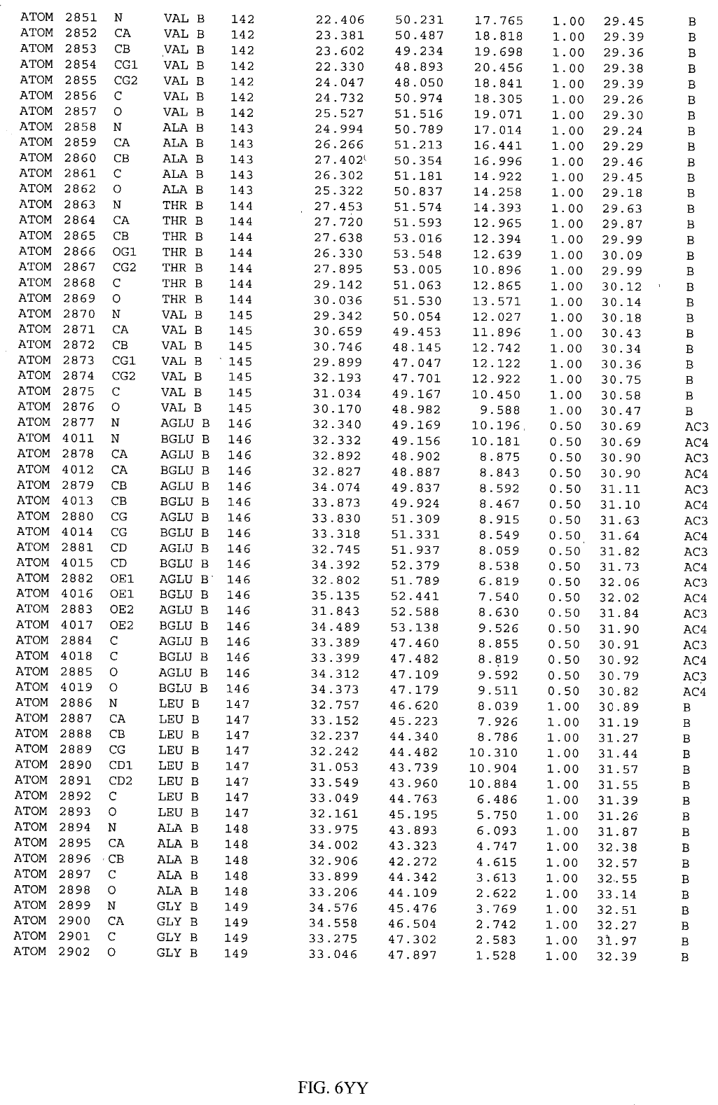

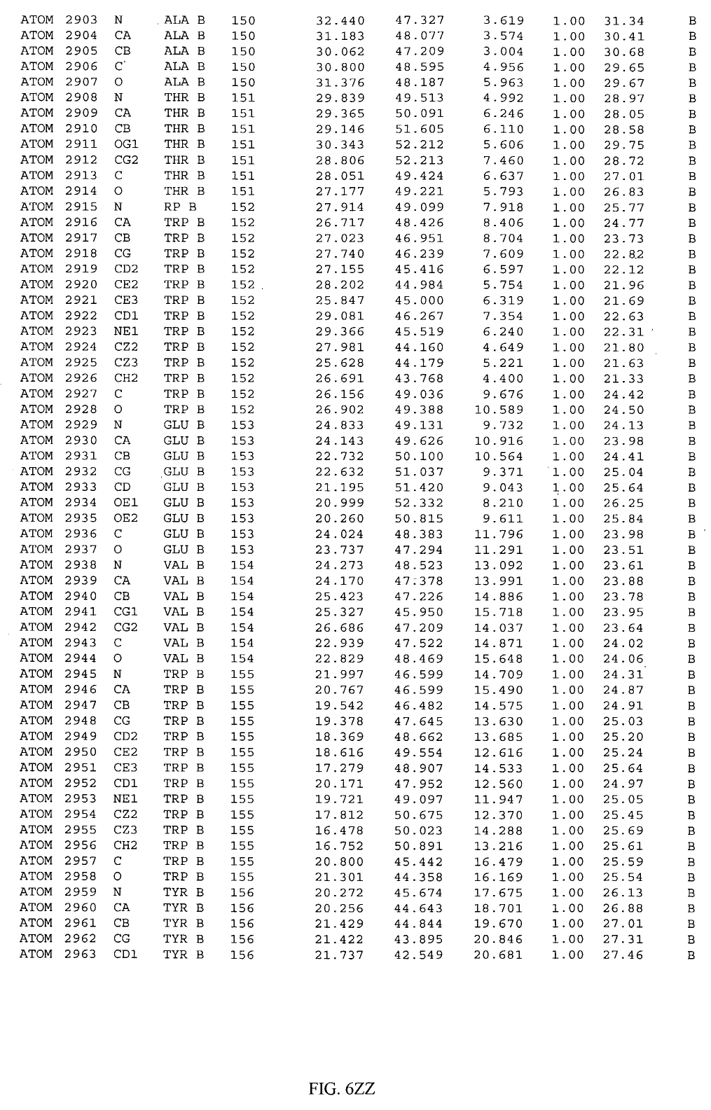

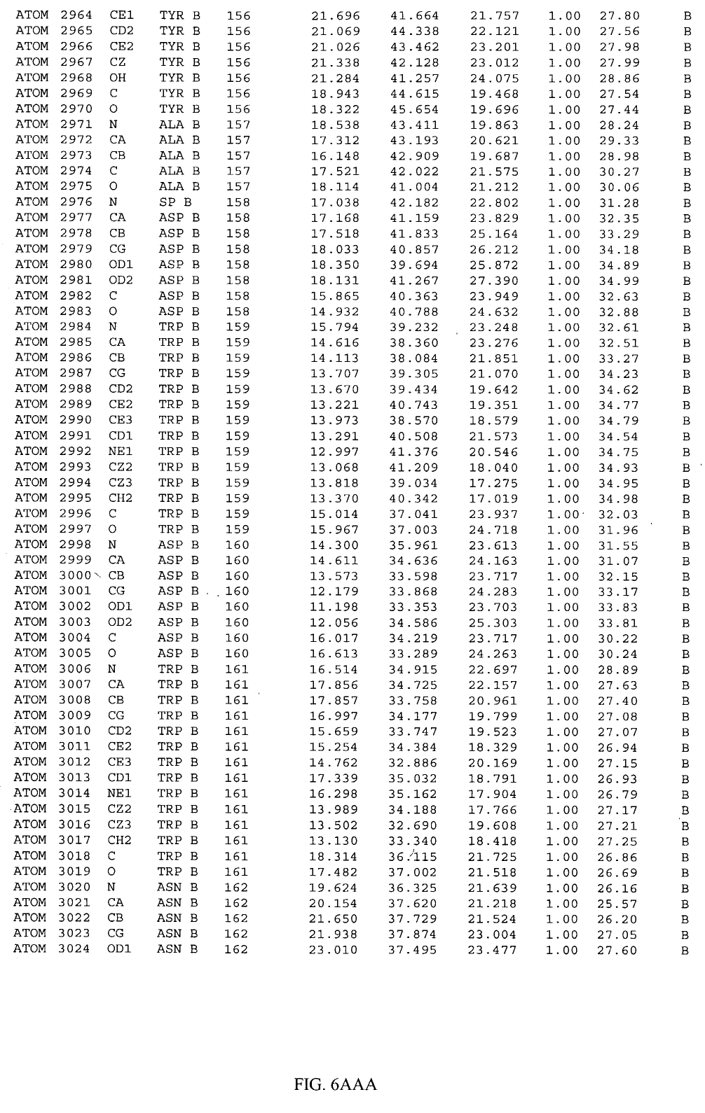

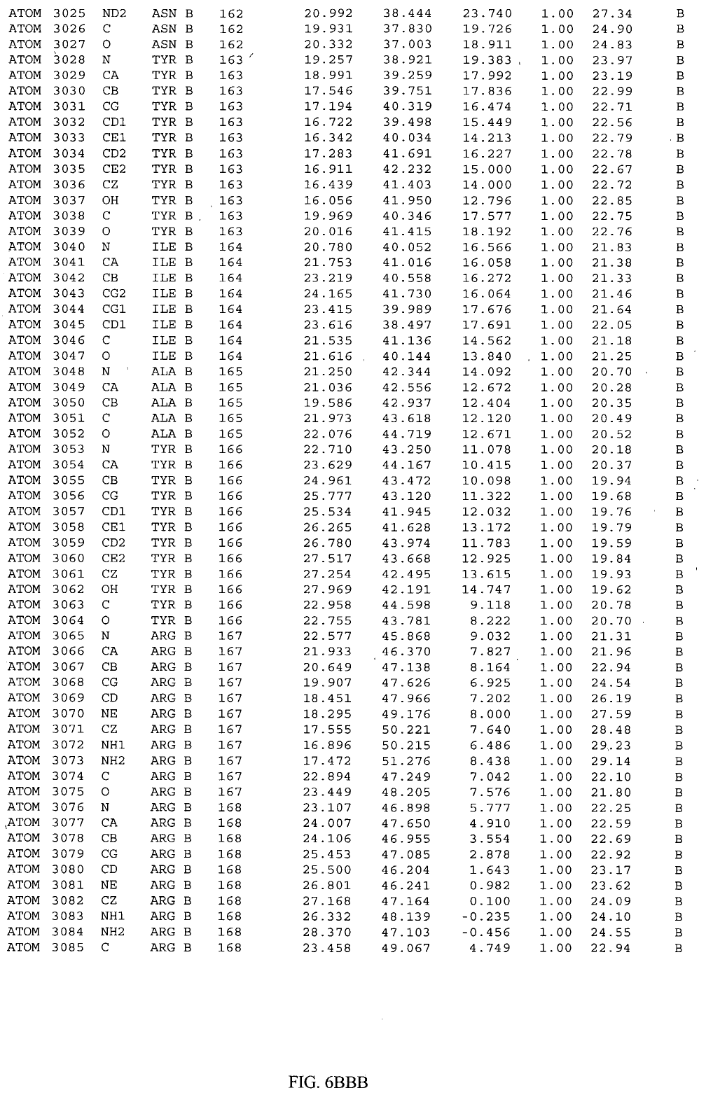

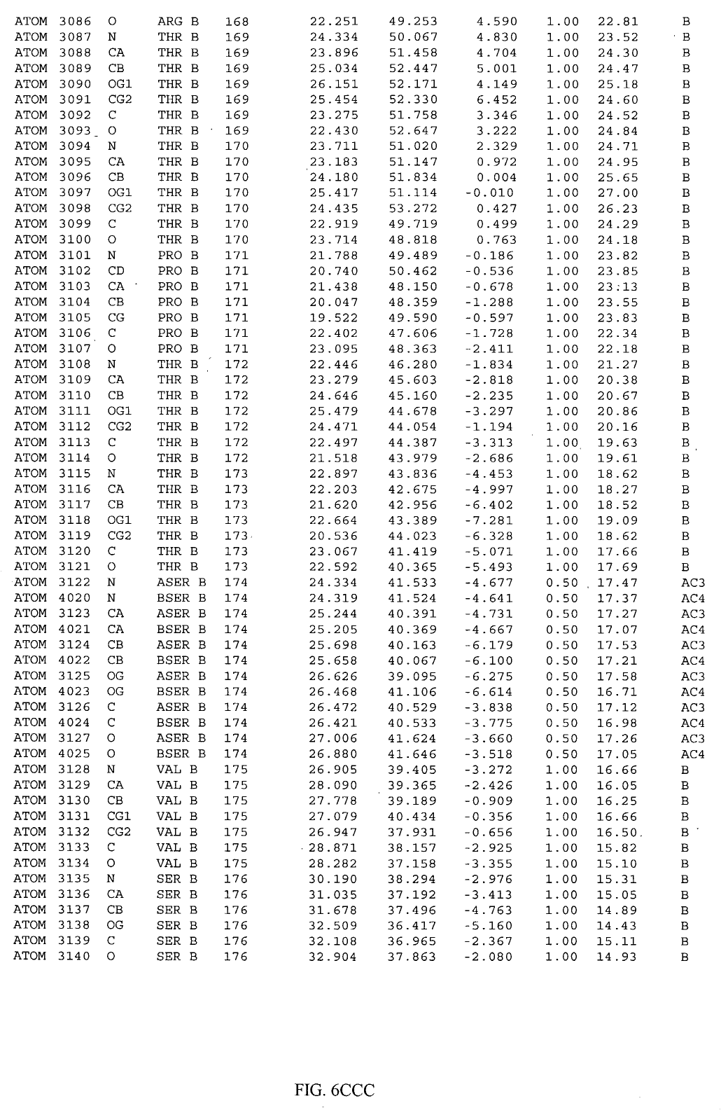

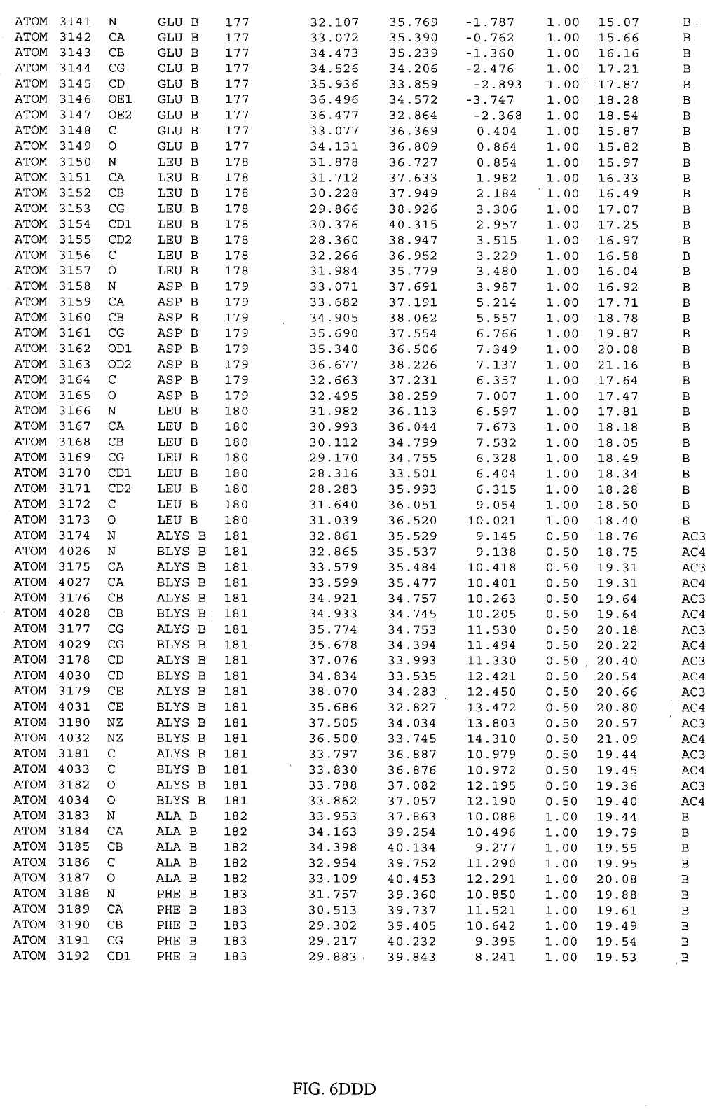

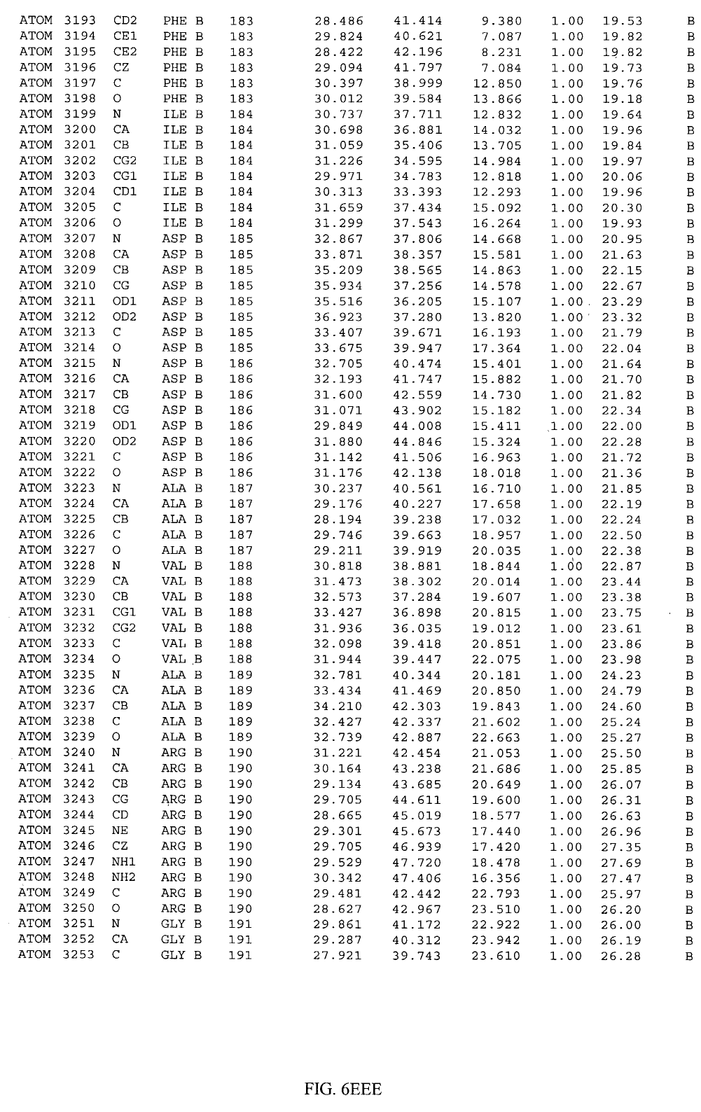

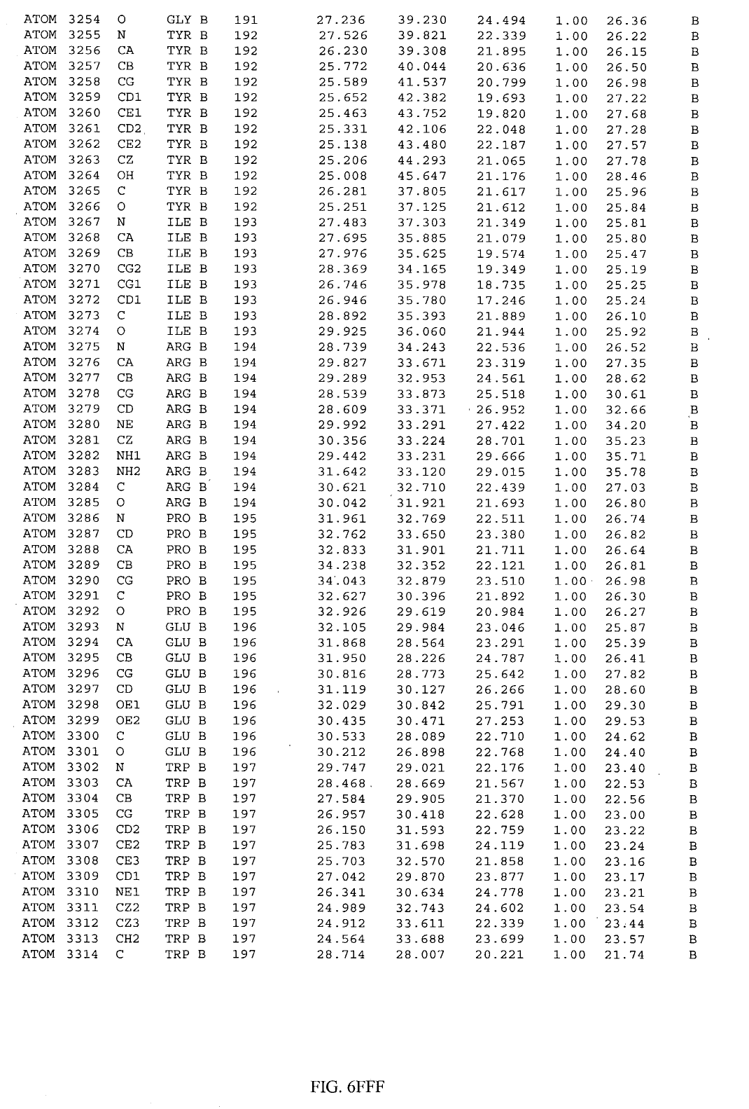

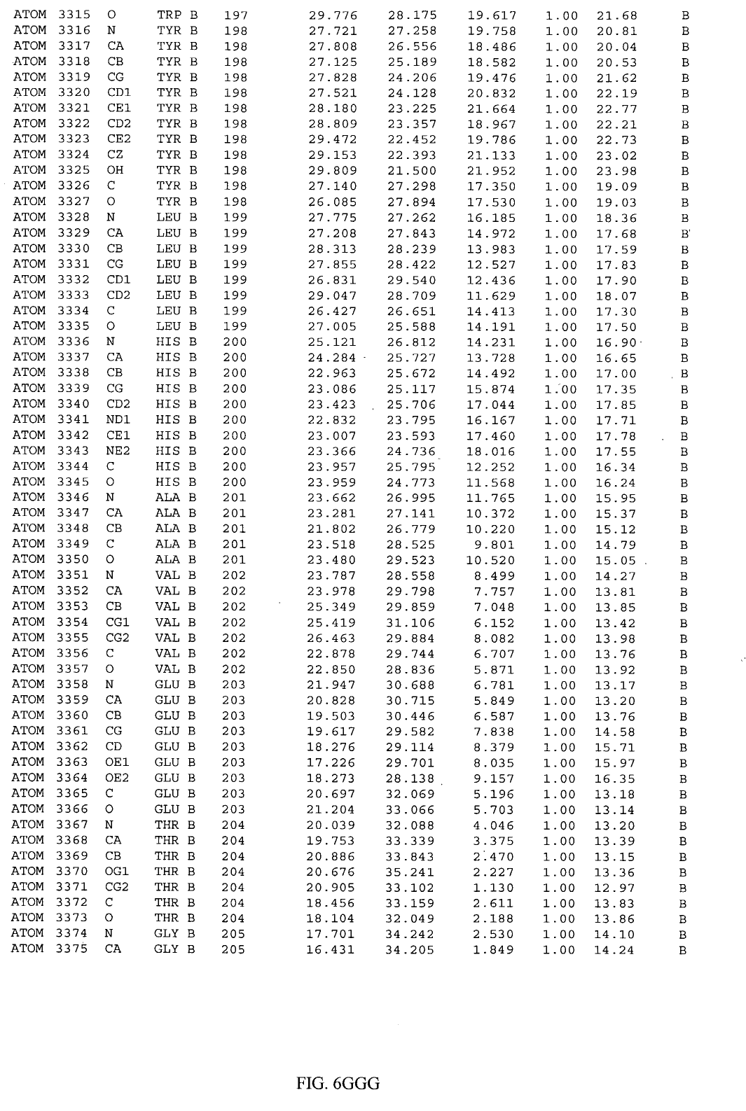

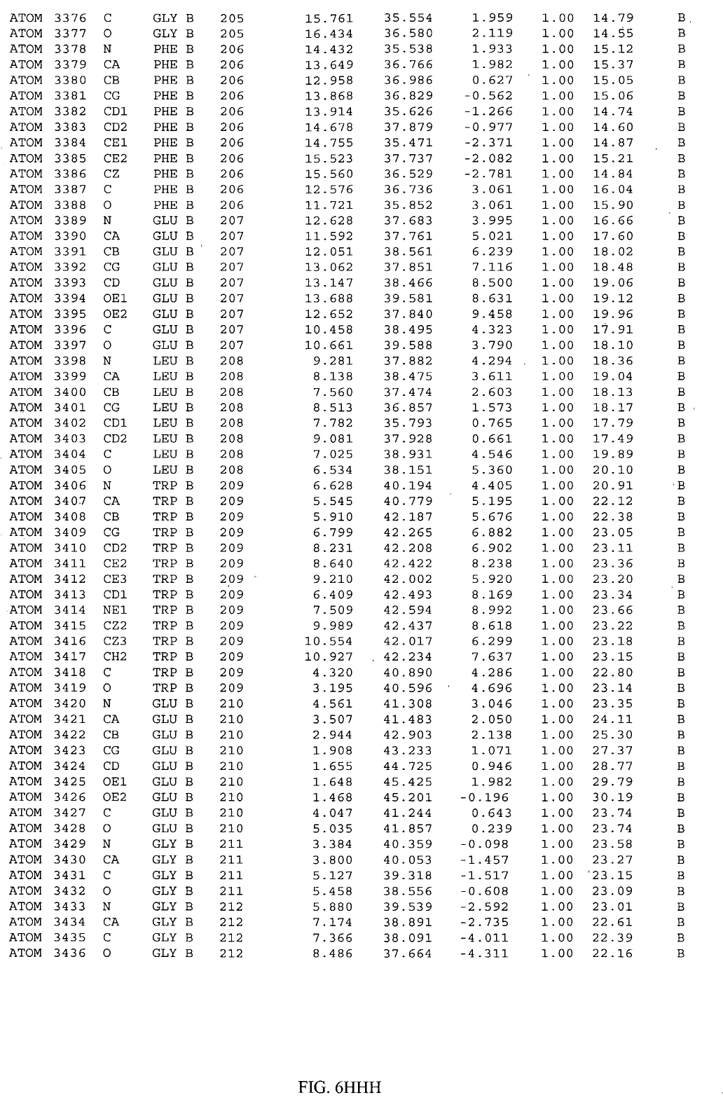

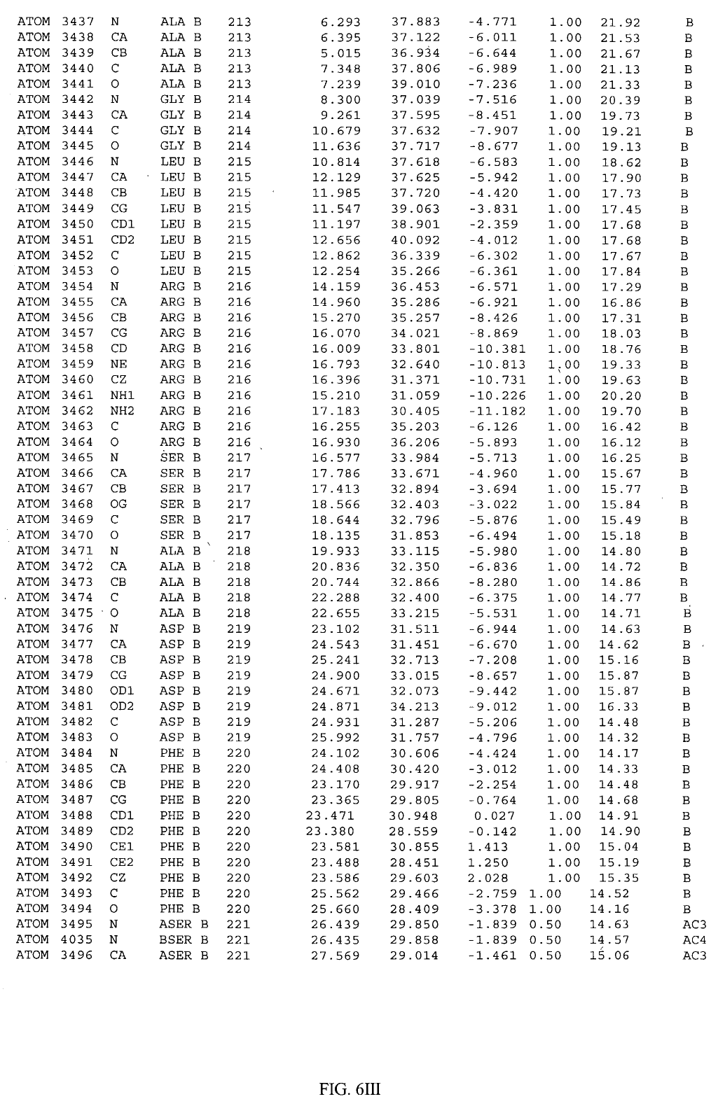

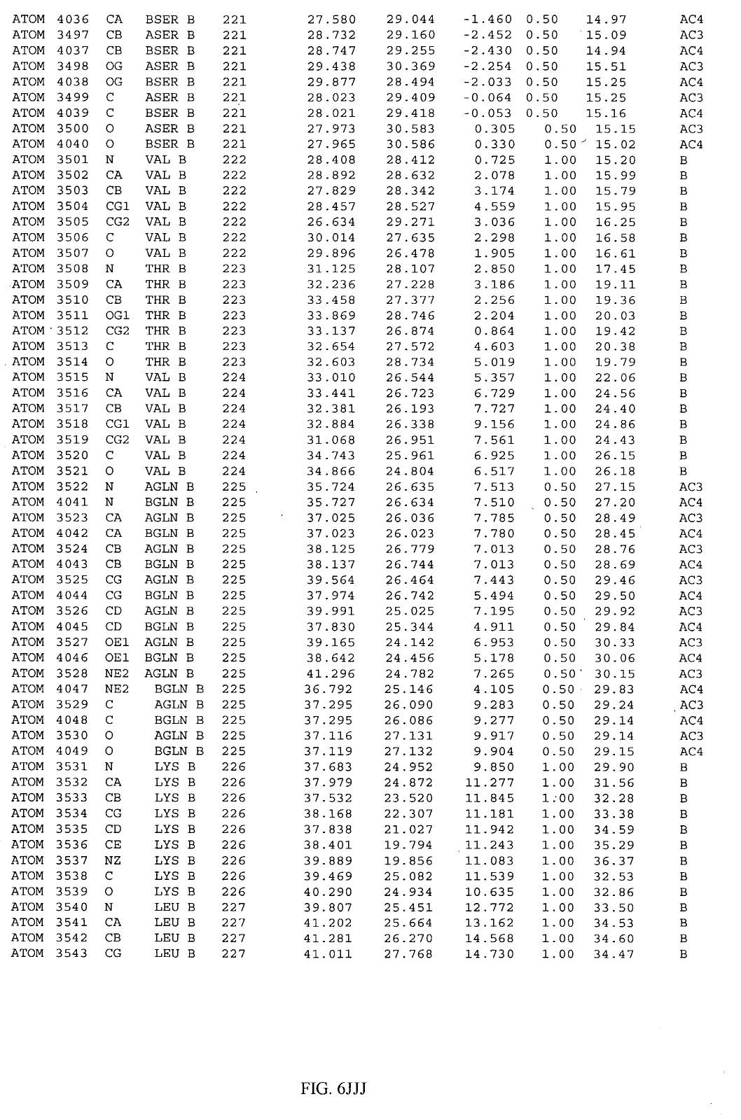

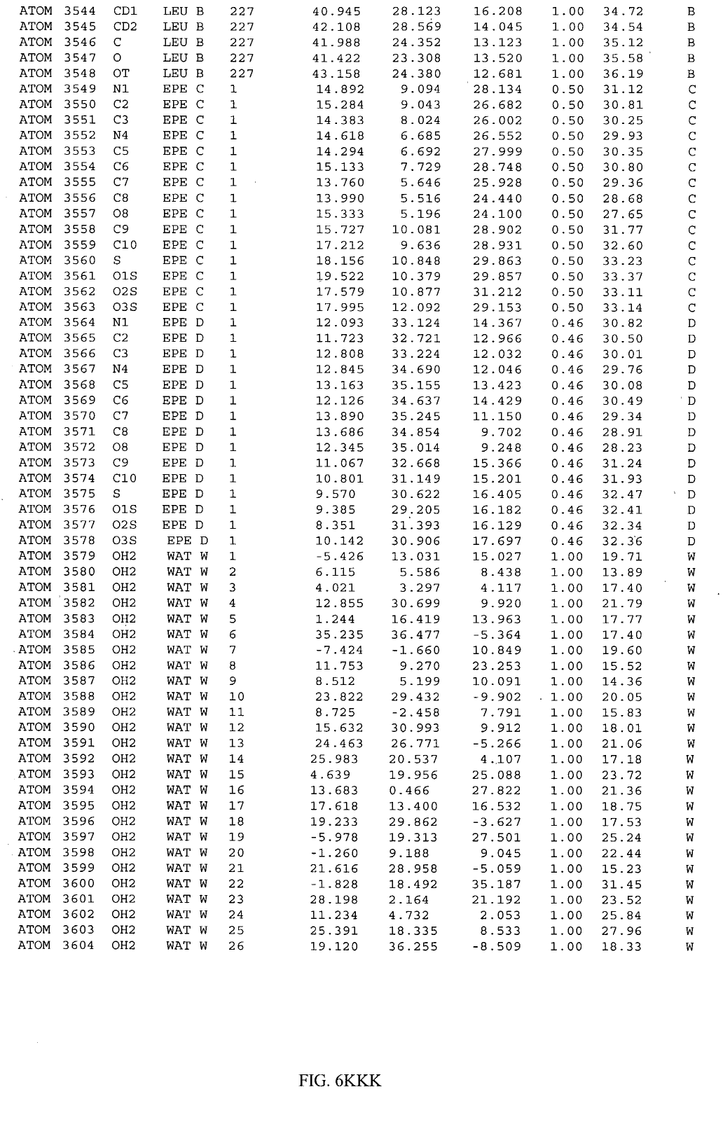

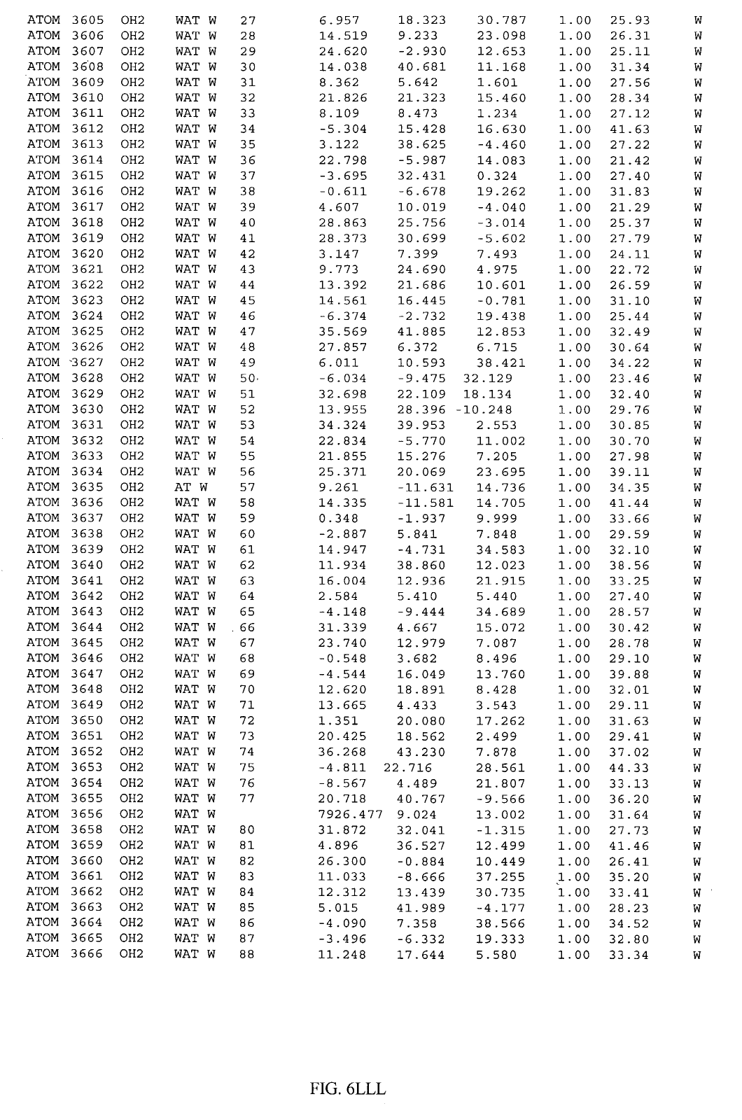

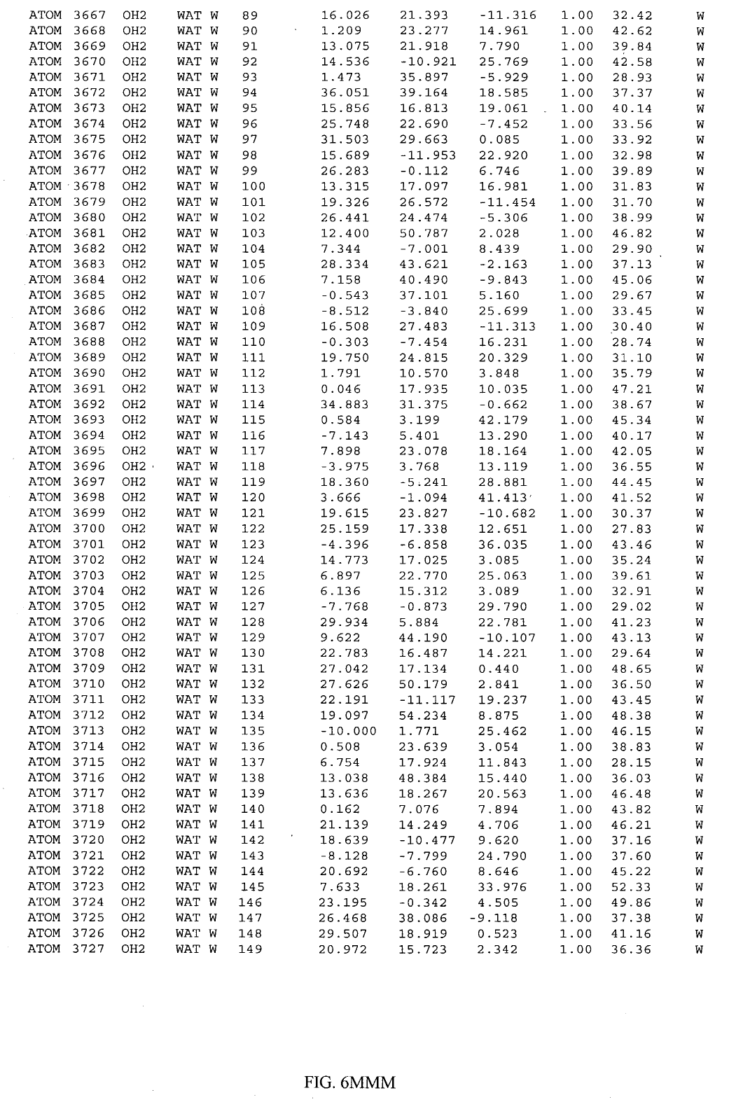

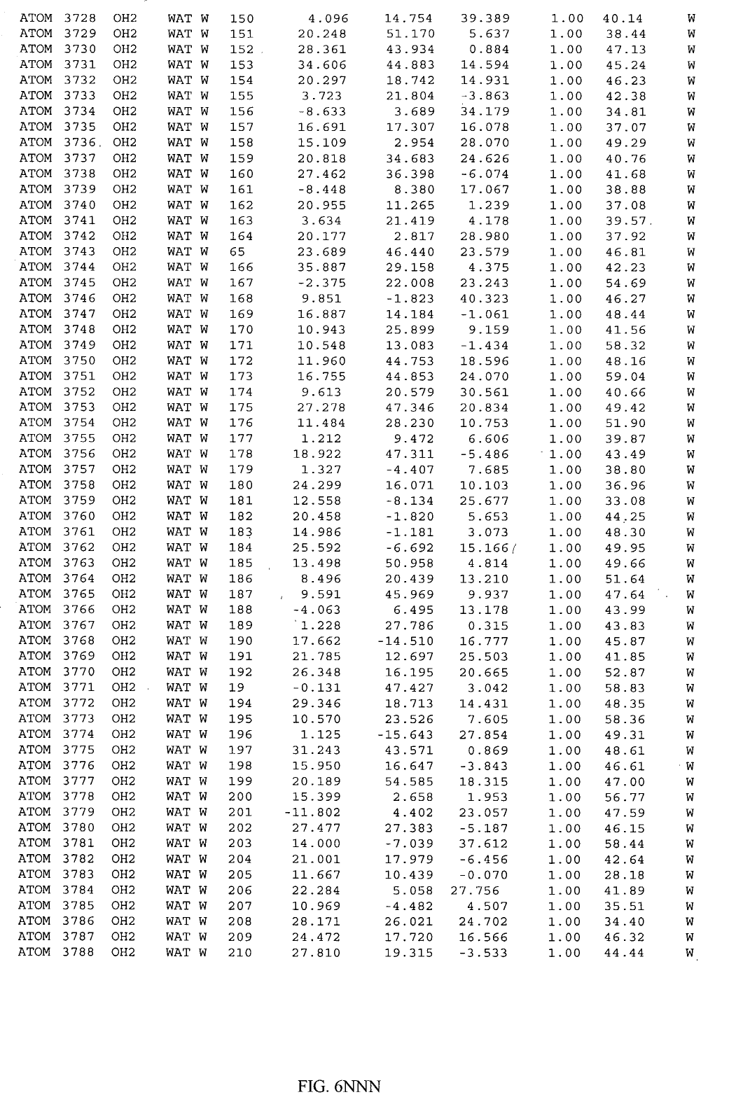

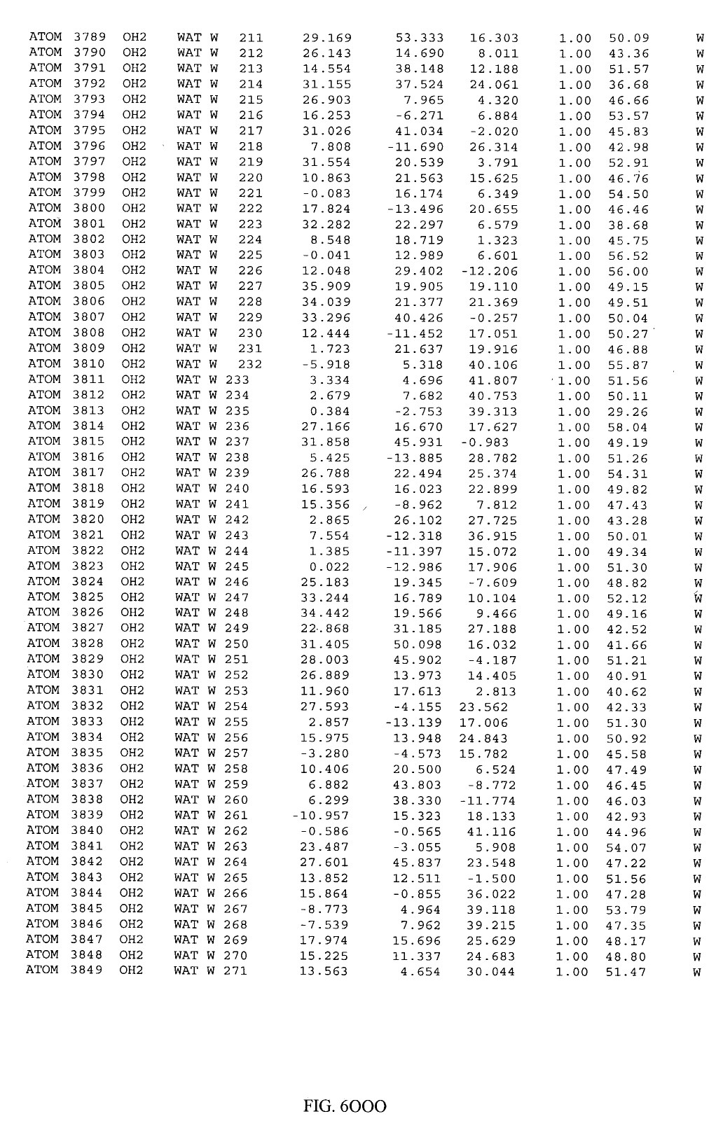

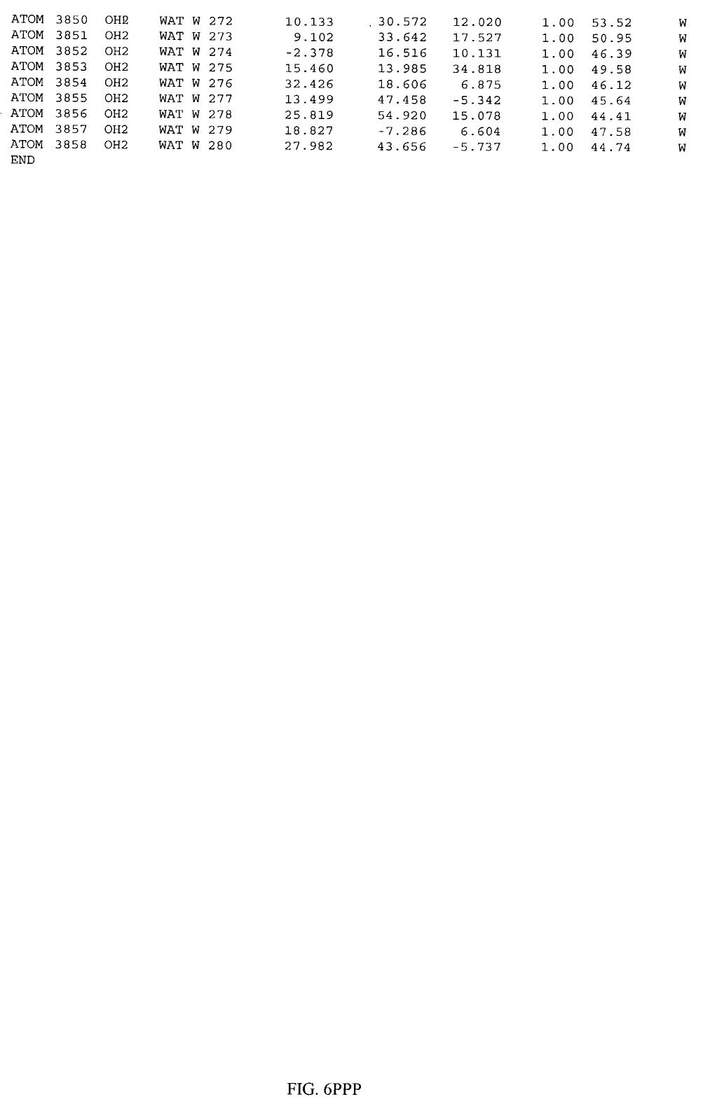

The three-dimensional structure of the Cel12A cellulase from Rhodothermus marinus is disclosed in Example 1 below and the structural coordinates are set forth in FIGS. 6A-PPP.

Protein structure can be analyzed by a variety of methods to determine various structural features and characteristics. In Example 1 below, hydrogen bonds and ion pairs were identified using the CCP4 program CONTACT (Collaborative Computational Project, Number 4, 1994) with cut-off distances of 3.2 Å for hydrogen bonds and 4.0 Å for strong ion pairs, although those possible ion pairs less than 6 Å or 8 Å were also calculated to detect possible ion pair networks. The percentage of polar surface was calculated using the default parameters in the program GRASP (Nicholls et al., 1991), and the secondary structure defined by the Kabsch and Sander criteria as implemented in PROCHECK where H and G were considered as helices and E or B as strands (Laskowski et al., 1993). Cavities were identified by VOIDOO (Kleywegt & Jones 1994) using a 1.2 Å probe. Structural analysis and comparison with other known structures can be peformed using a graphics display program such as the program O (Jones et al., 1991). In Example 1 below, structure superimpositions (structural alignments), in which 3-dimensional structures are superimposed according to structurally conserved regions, were carried out using LSQMAN (Kleywegt 1996).

The structure of Rhodothermus marinus Cel12A provided with the invention is the first structure of a thermophilic member of glycosyl hydrolase family 12 to have been solved. As outlined in Example 1 below, the structure has identical topology to those of the other two known structures of members of this enzyme family, both of which are of mesophilic enzymes. The comparison with the structures of the homologous mesophilic enzymes reveals several unique features of the structure of the Rhodothermus marinus cellulase provided by the invention. The structural similarity (and dissimilarity) between this cellulase and the mesophilic enzymes serves to highlight features that possibly contribute to its thermostability. For example, the present structure exhibits a vast increase in ion pair number and a considerable stabilization of a mobile region seen in S. lividans CelB2. Additional aromatic residues in the active site region could also contribute to the difference in thermophilicity. Some of the unique structural features of the structure provided by the invention are shared with other related thermostable enzymes as indicated by sequence comparison.

As outlined in Example 1 below, electrostatic interactions are increasingly favourable for stabilization at higher temperature and the higher occurrence of such interactions has been implicated as the most common stabilizing feature of hyperthermostable proteins (Vielle & Zeikus 2001). Many more ion-pairs are found in the present structure compared to the two structures of mesophilic origin (12 ion-pairs compared to 4 with a cut-off value of 4 Å) and the present structure is the only one with more extensive ionic networks of 4, 5 and 6 members. The high occurrence of ion pairs in the thermophilic structure is probably the most prominent feature contributing to overall stability, which correlates well with observations for other hyperthermostable proteins.

Many of the specific ion pairs are likely to be important for thermostabilization, and analogous ion pairs can be introduced in other homologous/structurally related proteins in order to improve their stability. The methods presented here can be used in a similar way to determine features likely to be important for thermostability in this family of proteins and used to guide modifications of other proteins. The structural information provided by the invention, including specific residues identified and listed herein, can also be used following sequence alignment to guide rational modifications in other related proteins in order to increase thermostability as exemplified in Example 2 below. The specific ion pairs that are found in the cellulase structure provided but not found in the previously known structures include: Glu4-Arg47, Arg8-Glu29, Asp10-Arg12, Asp10-Arg20, Asp13-Arg20, Glu35-Arg216, Arg47-Asp49, Asp51-Arg100, Arg79-Glu83, Arg80-Glu83, Asp86-Arg88, Arg88-Glu177, Arg88-Asp179, Arg100-Glu210, Arg141-Glu153, Glu153-Arg167, Lys181-Asp185, Asp186-Arg190, Arg194-Glu196 and Arg216-Asp219 (See, e.g., SEQ ID NO: 1). Based on this information and sequence alignment, the invention provides methods to modify any other protein of substantial structural similarity to the structure provided by the invention, in order to include one or more ion-pairs formed by residues at positions corresponding in position to the above-listed residues. Preferably such modifications include, e.g., having an Asp residue at a position corresponding to position 13 in the R. marinus cellulase Cel12A sequence and an Arg residue at position corresponding to position 20; also a Glu residue corresponding to position 4 and Arg residue corresponding to position 47; also an Arg residue corresponding to position 8 and Glu residue corresponding to position 29; also an Asp residue corresponding to position 10 and Arg residue corresponding to position 12; also an Asp residue corresponding to position 10 and Arg residue corresponding to position 20; also a Glu residue corresponding to position 35 and Arg residue corresponding to position 216; also an Arg residue corresponding to position 47 and Asp residue corresponding to position 49; also an Asp residue corresponding to position 51 and Arg residue corresponding to position 100; also a His residue corresponding to position 67 and Glu residue corresponding to position 203; also an Arg residue corresponding to position 79 and Glu residue corresponding to position 83; also an Arg residue corresponding to position 80 and Glu residue corresponding to position 83; also an Asp residue corresponding to position 86 and Arg residue corresponding to position 88; also an Arg residue corresponding to position 88 and Asp residue corresponding to position 179; also an Arg residue corresponding to position 100 and Glu residue corresponding to position 210; also an Arg residue corresponding to position 141 and Glu residue corresponding to position 153; also an Arg residue corresponding to position 167 and Glu residue corresponding to position 153; also a Lys residue corresponding to position 181 and Asp residue corresponding to position 185; also an Arg residue corresponding to position 190 and Asp residue corresponding to position 186; also an Arg residue corresponding to position 194 and Glu residue corresponding to position 196 and also an Arg residue corresponding to position 216 and Glu residue corresponding to position 219. One or more substitutions can thus be made to a protein of interest to obtain one or more ionic pairs corresponding in location to one or more of the above ionic pairs. Other substitutions at the positions just listed are also possible to form one or more ion pairs formed by other different residues but generally at the same locations. One non-limiting example would be to reverse the polarity of the residues corresponding to one or more of the ion pairs of the R. marinus cellulase, e.g., introducing an Arg residue or another positively charged residue at the position corresponding to Glu83, and a Glu residue or another negatively charged residue at a position corresponding to Arg79. Different combinations of residues can thus be introduced that lead to the formation of ion pairs at the specific positions and contribute to the overall stability of the particular protein.

The stabilization of a mobile loop and the conservation of the distinct structural character of this loop among known thermophilic protein in the family, provide a further rationale for thermostabilization of other related proteins through engineering of a corresponding loop region. The particular loop region in the sequence can thus be substituted with the corresponding region of the R. marinus cellulase sequence (approximately residues 155-165 in SEQ ID NO: 1). Additional point mutations can be made elsewhere in the sequence to accommodate the particular conformation of the loop region. An example of protein engineering of this kind is given in Example 2 below.

The invention is further illustrated by the following non-limiting examples:

### EXAMPLE 1

The structure of Rhodothermus marinus Cel12A at 1.8 Å Resolution.

Purification and Crystallization

Expression and purification of the cellulase Cel12A, mutated to remove the hydrophobic signal peptide and to add a C-terminal His-tag, were carried out as described previously (Wicher et al., 2001). The catalytic module of the cellulase was crystallized at 291K by the hanging drop vapour diffusion method, using a protein concentration of 14 mg/mL. The best quality crystals were obtained in 48 h from 0.1 M HEPES, pH 7.5, 20% w/v PEG 10000 (condition number 28 of Structure screen 2 from Molecular Dimensions Ltd.), and grew to dimensions of 1.7×0.4×0.3 mm.

Room temperature X-ray data were collected from a single crystal using a MAR Research MAR300 imaging-plate detector mounted on a Rigaku RU-H3R X-ray generator with MSC/Osmic (Blue) confocal mirror assembly, operating at 50 kV, 100 mA. Data were processed and scaled using DENZO and SCALEPACK (Otwinowski and Minor 1997); data collection parameters are summarised in Table 1. For cross validation purposes 5% of the reflections were set aside, the same set of free reflections being used in all subsequent refinement steps.

Structure Solution and Refinement

Initial phases were obtained by the molecular replacement method, using the structure of the mesophilic Streptomyces lividans cellulase as a search model (1n1r, Sulzenbacher et al., 1997), with the program AMoRe (Navasa 1994). Solutions for two molecules were found and the space group was unambiguously assigned to P2₁2₁2₁. The initial map calculated from a polyalanine reduction of this solution was improved using the program wARP (Perrakis et al., 1997, van Asselt et al., 1998). Automatic tracing and model building within wARP yielded only 202 residues of a possible 452, but the quality of the wARP map allowed the majority of the remainder to be built using the graphics program O (Jones et al, 1991). A section of about twenty residues from residue 68 was observed to have been incorrectly sequenced; the correct sequence is given in FIG. 2 and SEQ ID NO: 1. Simulated annealing refinement was carried out using the program CNS, (Brunger et al., 1998) including a bulk solvent correction. Model refinement cycles involved maximum likelihood refinement in CNS, followed by automatic solvent generation in wARP, visual solvent checking, and then model rebuilding in O. An area of extra density in the active site was interpreted as a HEPES molecule, which can be disordered in the crystal, but at this resolution only one conformer was clearly defined.

Analysis of the Model

Hydrogen bonds and ion pairs were identified using the CCP4 program CONTACT, (Collaborative Computational Project, Number 4, 1994) with cut-off distances of 3.2 Å for hydrogen bonds and 4.0 Å for strong ion pairs, although those possible ion pairs less than 6 Å or 8 Å were also calculated to detect possible ion pair networks. The percentage of polar surface was calculated using the default parameters in the program GRASP (Nicholls et al., 1991), and the secondary structure defined by the Kabsch and Sander criteria as implemented in PROCHECK where H and G were considered as helices and E or B as strands (Laskowski et al., 1993). Cavities were identified by VOIDOO (Kleywegt & Jones 1994) using a 1.2 Å probe. Structure superimpositions were carried using LSQMAN (Kleywegt 1996).

TABLE 1
X-ray data collection and model refinement statistics.

| Data collection | |
|---|---|
| Space Group | P2₁2₁2₁ |
| Unit cell dimensions (Å) | |
| a | 56.10 |
| B | 67.78 |
| C | 132.26 |
| Resolution (Å) (last shell) | 1.80 (1.86-1.80) |
| Number of observations | 626655 |
| Number of unique reflections | 47645 |
| Completeness overall (last shell) | 93.4 (88.1) |
| R merge (%) overall (last shell) | 7.4 (27.5) |
| Average I/σI overall (last shell) | 27.7 (5.3) |
| Refinement | |
| Resolution range (Å) | 30-1.8 |
| Number of reflections in working set | 42154 |
| Number of reflections in test set | 2258 |
| Number of protein atoms | 3739 |
| Number of ligand atoms | 30 |
| Number of solvent atoms | 279 |
| Average B value (Å²) | |
| Protein main chain (A:19.1, B:21.9) | 20.5 |
| Protein side chain (A:20.2, B:22.8) | 21.5 |
| Ligand | 30.9 |
| Water | 38.9 |
| R-cryst (%) | 17.3 |
| R-free (%) | 19.4 |
| Residues in Ramachandran core region | 91.9 (8.1) |
| (additionally allowed) % | |
| Rms deviation from ideal geometry | |
| Bonds (Å) | 0.005 |
| Angles (°) | 1.34 |

TABLE 2
Comparison of features likely to contribute to thermostability
in family 12 cellulases. For ion pairs, the count is for number
of single charge-to-charge interactions and the values in
parentheses are the values excluding bonds involving His
residues and terminal carboxyl- and amino groups.

| | | R. marinus | S. lividans | T. reesei |
|---|---|---|---|---|
| | | Cel12A | CelB2 | TrCel12A |
| Ion pairs | <4Å | 12 (11) | 4 | 4 (2) |
| | <6Å | 16 (15) | 7 | 4 (2) |
| | <8Å | 24 (22) | 10 | 6 (4) |
| Amino acids | Asn + Gln | 16 | 21 | 35 |
| | Ser + Thr | 32 | 47 | 46 |
| | Pro | 7 | 14 | 7 |
| | Gly | 25 | 24 | 27 |
| | Cys | 4 | 4 | 2 |
| | Phe + Tyr + | 30 | 25 | 33 |
| | Trp | | | |
| Polar surface | | 76% | 70% | 73% |
| Hydrogen bonds | | 227 | 201 | 199 |
| Secondary structure | % α | 6.6 | 6.3 | 7.7 |
| | % β | 60.4 | 57.5 | 59.6 |
| Number of cavities | | 1 | 1 | 1 |
| Volume of | | 1.9 | 8.4 | 4.5 |
| cavity Å³ | | | | |

TABLE 3
A comparison of substrate binding residues in Cel12A,
CelB2 and TrCel12A, and the correlation of the residues
in Cel12A relative to the unliganded or complexed
CelB2 where these differ.

| | | | | |
|---|---|---|---|---|
| | | | | Position of |
| role | Cel12A | CelB2 | TrCel12A | Cel12A residues |

−3 subsite

| | | | | |
|---|---|---|---|---|
| Stacking | Trp 68 | Tyr66 | Tyr 111 | unliganded |
| | Trp 9 | Phe 8 | Trp 7 | complex |
| Binds O6 | Asn 24 | Asn 22 | Asn 20 | |

−2 subsite

| | | | | |
|---|---|---|---|---|
| Stacking | Trp 26 | Trp 24 | Trp 22 | complex |
| Binds O2 | Asn 24 | Asn 22 | Asn 20 | |
| Binds O3 | His 67 | His 65 | — | |

−1 subsite

| | | | | |
|---|---|---|---|---|
| Nucleophile | Glu 124 | Glu 120 | Glu 116 | complex |
| Stabilizes | Trp 161 Nε | Asn 158 | — | Nε closer |
| nucleophile | | | | to cpmplex |
| Maintains | Asp 106 | Asp 104 | Asp 99 | complex |
| charge | | | | |
| Nucleophilic | | | | complex |
| H₂O | | | | |
| Catalytic | Glu 207 | Glu 203 | Glu 200 | |
| acid/base | | | | |
| Stabilizes | Asn 102 | Asn 100 | Asn 95 | |
| acid/base | | | | |
| Possible + 1 | Met 126 | Met 122 | Met 118 | complex |

Quality of the Final Model

The final Cel12A model contains two molecules, each comprising residues 2 to 227 of the possible 247, thereby covering the whole native catalytic domain but excluding the C-terminal tag, which is disordered in the crystal, together with 280 water molecules and two HEPES solvent molecules. Twelve residues are modelled with dynamic disorder; these all lie on the outside of the molecule, either in loop regions (residues 29, 54, 73, 74, 100, 146, 173), or regions of close inter-molecular contact (residues 12, 114, 117, 120). None is within the active site cleft.

This model gives an R-factor of 17.3% (no ai cut-off) and an R-free of 19.4% (for 5% of data), with good stereochemistry indicated by root mean square deviations from ideal geometry of 0.005 Å in bond lengths and 1.34+ in bond angles. Monomers A and B have mean isotropic B-values of 19.6 Å² and 22.3 Å² respectively, these relatively high values being due to the room temperature data collection. The two molecules are very similar having an RMSD over all C_{α} atoms of 0.184 Å. A Ramachandran plot (Ramakrishnan & Ramachandran, 1965), calculated by PROCHECK (Laskowski et al., 1993), indicated that 92.2% of the non-glycine residues fall in the most favoured region, with none in the “generously allowed” or disallowed regions. Each monomer has a cis-Proline, Pro 78.

Overall Structure

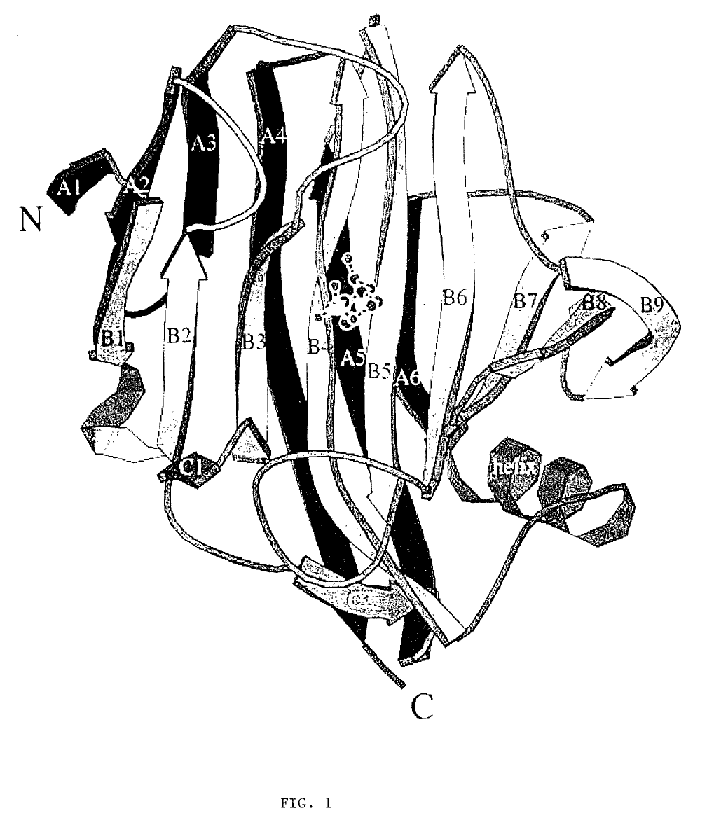

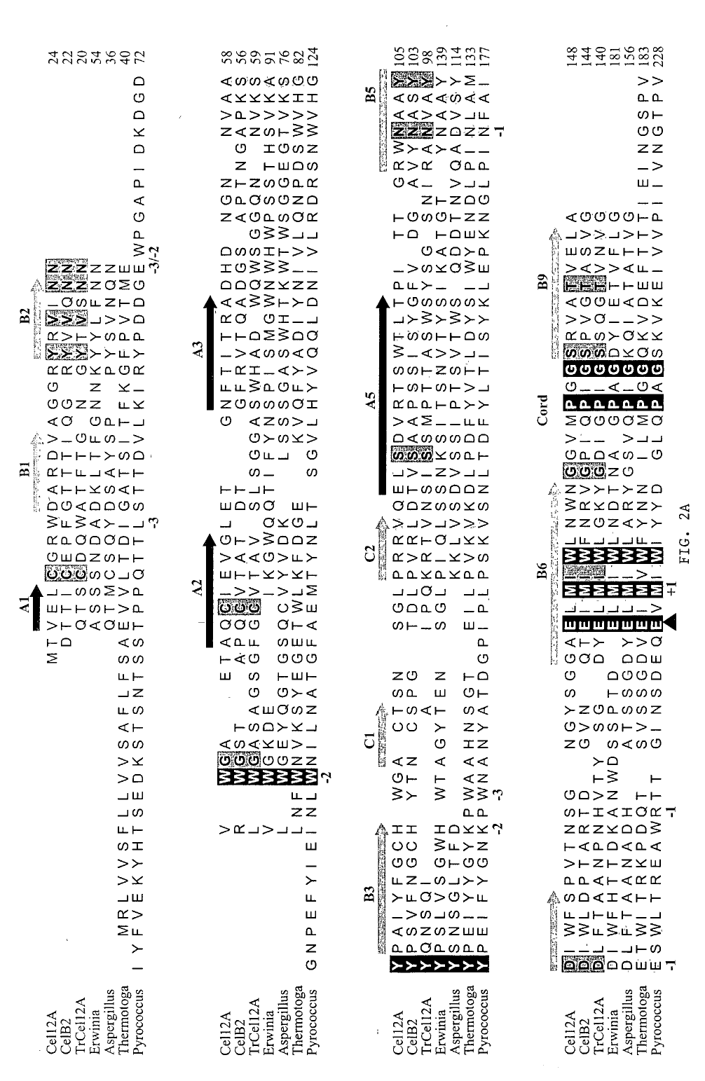

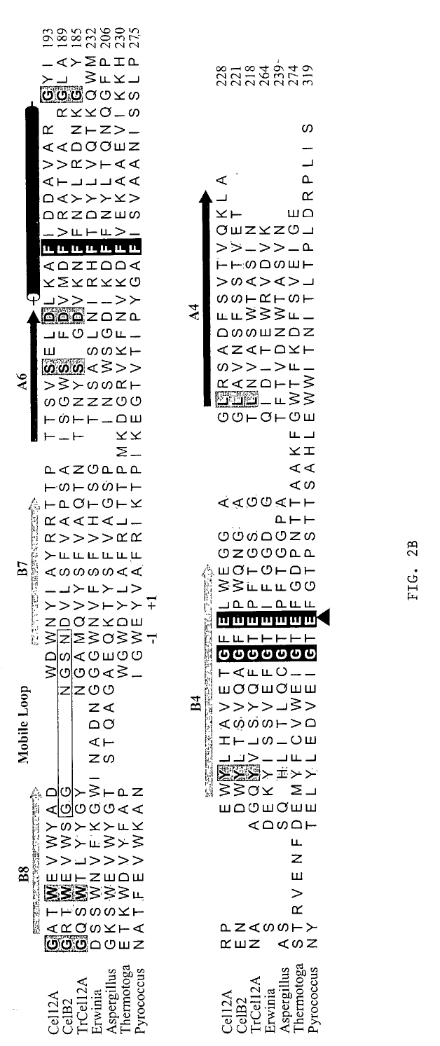

Rhodothermus marinus Cel12A folds into a single domain of two β-sheets that pack against one another (FIG. 1). The outer sheet, A, has six anti-parallel β-strands, while the inner sheet, B, has nine β-strands, mostly antiparallel. Sheet B curves to form the active site cleft on the inside, while the convex side of the sheet forms hydrophobic interactions with sheet A and with the helix. The overall architecture is that of the classic β-jelly roll, similar to that of the cellulases from Streptomyces lividans CelB2 and Trichoderma reesei TrCel12A, and closely resembling the topology of the glycosyl hydrolase family 11 xylanases. The dimensions of the enzyme are approximately 40 Å×40 Å×3 Å

The Cel12A catalytic residues were identified by sequence and structural comparison with the other cellulases and lie on sheet B within the cleft, Glu 207 on strand B4 and Glu 124 on B6, topologically equivalent to the well-characterized catalytic glutamic acid residues in the previous structures. In CelB2 the catalytic residues were identified initially through analogy with the xylanase family 11 structures, then subsequently through the structure of a trapped glycosyl-enzyme intermediate. This assignment was confirmed by kinetic analysis of S. lividans CelB2 (Zechel et al., 1998). The catalytic residues in Trichoderma reesei TrCel12A were confirmed by site-directed mutagenesis (Okada et al., 2000). In Cel12A the acid-base Glu 207 forms a strong hydrogen bond to Asn 102, (conserved or conservatively substituted by Asp in the family 12 cellulases) while the nucleophile, Glu 124, interacts with Asp 106 (conserved or substituted by Glu) and with Trp 161. This last interaction is different from that seen in the other cellulase structures (see mobile loop discussion below). A striking feature of the rest of the substrate-binding cleft is the large number of solvent-exposed aromatic amino acids, in particular tryptophan side chains, which line the cleft. In order to identify the roles of these residues, a comparison with the structure of Streptomyces lividans CelB2 with a covalently-bound intermediate was undertaken.

Comparison with Other Cellulase Structures

The overall topology of Cel12A is very similar to that of the mesophilic family 12 cellulases, a simple rigid-body least squares algorithm giving an rmsd of 1.21 Å for 218 equivalent C_{α} atoms in the apo structure of CelB2, (PDB entry 1n1r) (Sulzenbacher et at., 1997) and 1.50 Å for 204 C_{α} atoms in TrCel12A (1h8v) (Sandgren et al., 2001). As might be expected from the lower rmsd and higher sequence identity (34% with CelB2, 28% with TrCel12A), more structural features are conserved between Cel12A and CelB2 than between Cel12A and TrCel12A. For instance, topologically identical disulphide bonds connect Cys 6 on strand A1 with Cys 33 on strand A2 (CelB2 Cys 5 and 31) and Cys 66 (64) with Cys 71 (69) hold together the two short strands in sheet C. TrCel12A contains the first but not the second disulphide bond. Examples of enzymes lacking disulphide bonds are also known within the GH-C clan, demonstrating that they are probably not needed for the overall fold, but rather as suggested by Sandgren (2001) for local stabilization. Both bacterial cellulase structures also have a cis proline (Pro 78, Cel12A numbering) in the loop between B5 and A3, which is absent in the fungal structure.

The structures of the R. marinus Cel12A cellulase and the S. lividans CelB2 cellulase were superimposed to determine root mean square deviation in a well conserved core of the enzymes. The Cα—atoms in the following residues were superimposed:

| R. marinus Cel12A | S. lividans CelB2 |
|---|---|
| 18-26 | 16-24 |
| 31-37 | 29-35 |
| 56-64 | 54-62 |
| 84-95 | 82-93 |
| 99-112 | 97-110 |
| 122-142 | 118-138 |
| 149-157 | 145-153 |
| 161-173 | 158-170 |
| 196-210 | 192-206 |
| 215-224 | 211-220 |

The root mean square deviation between these Cα atoms was 0.842 Å.

As in these previous cellulase structures, in Cel12A the active site is 35 Å in length, which is longer than in the family 11 xylanases due to the extension of the loop between B3 and A5 (including the short C p strands), which may form part of the −3 or −4 binding site (the sugar-binding subsite nomenclature is that where subsites are labeled from −n at the non-reducing end to +n at the reducing end, with cleavage between −1 and +1; Davies et al., 1997). A second long loop, that between A3 and B3, provides much of the wall of the central part of the active site, forming a 15 Å deep cleft in both bacterial cellulases, more open than in TrCel12A where the loop is shorter. The B8-B7 loop is shorter in Cel12A, making the catalytic cleft slightly wider, 9 Å, than in the other cellulases. Although the structures are topologically similar, only ten amino acids are conserved across the spectrum of cellulases from family 12 (FIG. 2). These include the catalytic glutamic acids, (Glu124, Glu207), a methionine and tryptophan (Met 126 and Trp 26) thought to interact with the +1 and −2 sugars respectively, and a tyrosine and tryptophan (Tyr57 and Trp 128) that lie at the base of the catalytic cleft. Phe 183 at the N-terminus of the helix forms an aromatic cluster with Tyr 166 and Trp 152 (aromatic residues throughout the family), which strengthens the predominantly hydrophobic interaction between the helix and the two β-sheets.

Within the family 12 cellulases, the Cel12A primary structure showed, despite its thermostability, slightly higher sequence identities to cellulases from mesophilic Streptomyces species, than to the thermostable cellulases. A major difference from the Streptomyces enzymes, is the absence of the CBM, but this module is also absent in TrCel12A, and the thermostable representatives from Pyrococcus and Thermotoga, so it is not an exclusively thermostabilizing feature. As might be expected from the relatively low sequence identity (34% with CelB2, 28% with TrCel12A), there are many differences between the structures. For instance, in CelB2 the sole lysine, (Lys 55), on strand B3 is buried and, due to the formation of strong hydrogen bonds with main chain atoms on A3 and A4, it has been suggested to play a “crucial” role in binding the sheets together (Sulzenbacher et al., 1997). In TrCel12A this lysine is conserved (Lys 58), and fulfils the same role, although it interacts with different residues on A3 and A4. However, in the more thermostable Cel12A, this position is occupied by an alanine, and other polar interactions in the vicinity are within the sheets, so the polar interaction between the two sheets in this region is not essential for thermostability. Another example is the valine residue on strand B7 (Val 160 in TrCel12A, Val164 in CelB2) proposed to be completely conserved in clan GH-C (Sandgren et al., 2001), but in Cel12A, and in the other thermostable representatives (see FIG. 2), found to be replaced by an Arginine (Arg 167). This forms one end of a three-member ion pair network, interacting with Glu 153 on B8 (also acidic in thermophilic sequences, and some mesophilic) and Arg 141 on B9, which is an Arg or Lys in the thermophilic sequences and hydrophobic or negatively charged in the mesophiles. This network is absent in the mesophilic structures and is part of the overall increase in polar surface seen in Cel12A (see below).

Active Site Comparison

Glycosyl hydrolase family 12 cellulases, or endoglucanases, hydrolyse β-1,4 linked glucans and “mixed linkage (β-1,3 and 1,4)” glucans with net retention of anomeric configuration. Within this broad classification Cel12A has been shown to hydrolyse soluble polysaccharides with β-1→4 and β-1→3.1→4 linkages (Carboxymethylcellulose (CMC), lichenan and glucomannan), but to have very low activity on Avicel and none on xylan or galactomannan (Wicher et al., 2001). S. lividans 66 CelB also hydrolyses CMC, acid-swollen Avicel but not xylan (Wittman et al.,1994), thus would appear to have a similar specificity to Cel12A. The specific function of TrCel12A has not been characterised (Sandgren et al., 2001). In the native state CelB has a second carbohydrate-binding domain. Although the protein is truncated in the catalytic domain for the structure determination, it remains active. Both Cel12A and TrCel12A lack the carbohydrate-binding domain, so the catalytic domain must carry out both cellulose binding and cleavage. (Wicher et al., 2001, Sandgren et al., 2001). As only the unliganded TrCel12A structure has been determined, the majority of the comparison below is concerned with CelB2 in the unliganded and ligand-bound forms. This comparison has more validity since in the larger differences between the two earlier structures, in particular the longer B3-A5 loop in CelB2 containing residues contributing to the −2 and −3 subsites and the shorter B2-A2 loop, the Cel12A structure more closely resembles the CelB2 than the TrCel12A structure.

Comparison with S. lividans CelB2

CelB2 has been co-crystallized with 2-deoxy-2-fluorocellotrioside (Sulzenbacher et al., 1999), which is commonly used to trap the covalent glycosyl-enzyme intermediate in retaining glycoside hydrolases. The structure reveals two species in the active site, both the intermediate and its hydrolysis product, 2-deoxy-2-fluorocellotriose, with the corresponding dual conformations of amino acid side chains in the −1 site. A comparison of the liganded and native CelB2 structures (2n1r and 1n1r) reveals small conformational changes in loops bordering the active site cleft and an rms difference of 0.42 Å over the structures as a whole. Cel12A has an rms deviation from the complexed CelB2 (2n1r) of 1.14 Å over 219 C_{α} atoms, which is less than with the unliganded CelB2 (1.21 Å over 218 C_{α} atoms), so it is overall more similar to the former. This was initially a surprise since inhibitors were not co-crystallized with Cel12A to cause a conformational change.

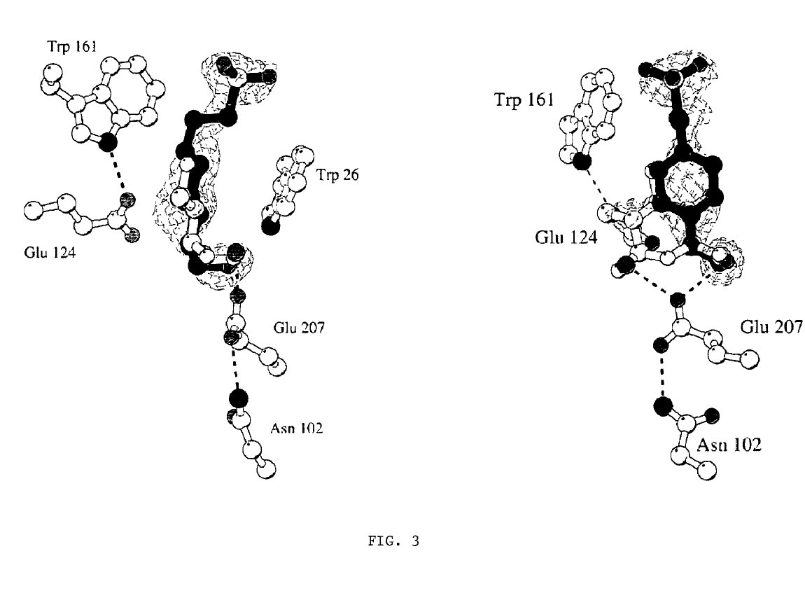

However, on comparison of the central −1 subsite it is clear that a HEPES buffer molecule lying in the Cel12A active site mimics a glucoside substrate (FIG. 3). The position of many side chains in Cel12A close to the HEPES were more similar to those in the complex CelB2 than in the native structure (Table 3), thus the Cel12A structure could represent an active configuration, at least in the central portion of the active site. The majority of substrate binding residues, identified by comparison with the CelB2 complex, are conserved (FIG. 2 and Table 3).

−3 and −2 Subsites

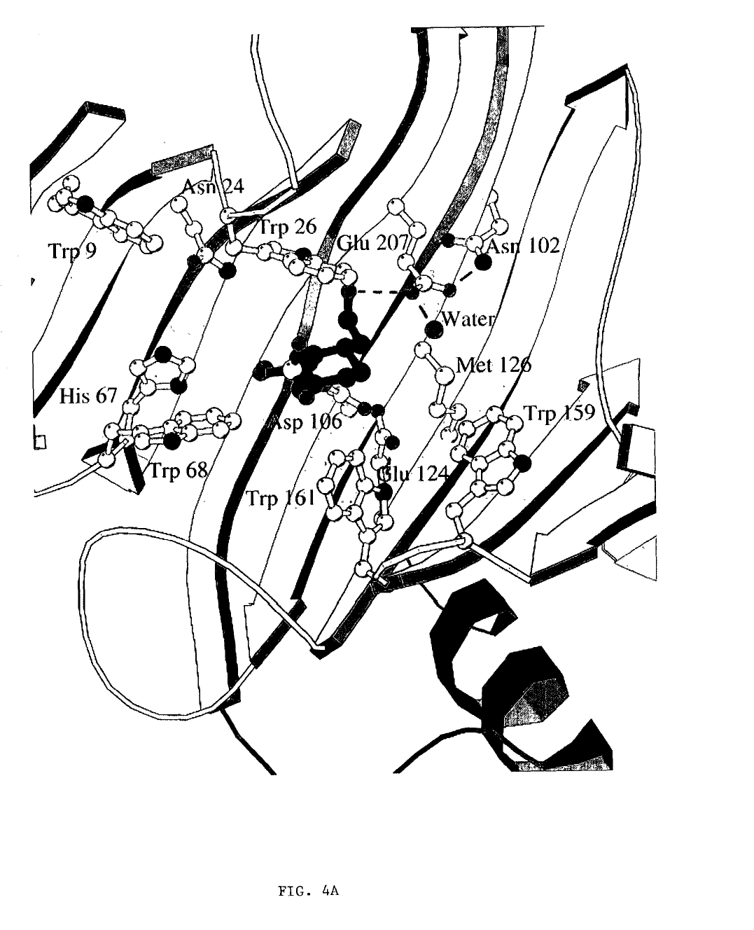

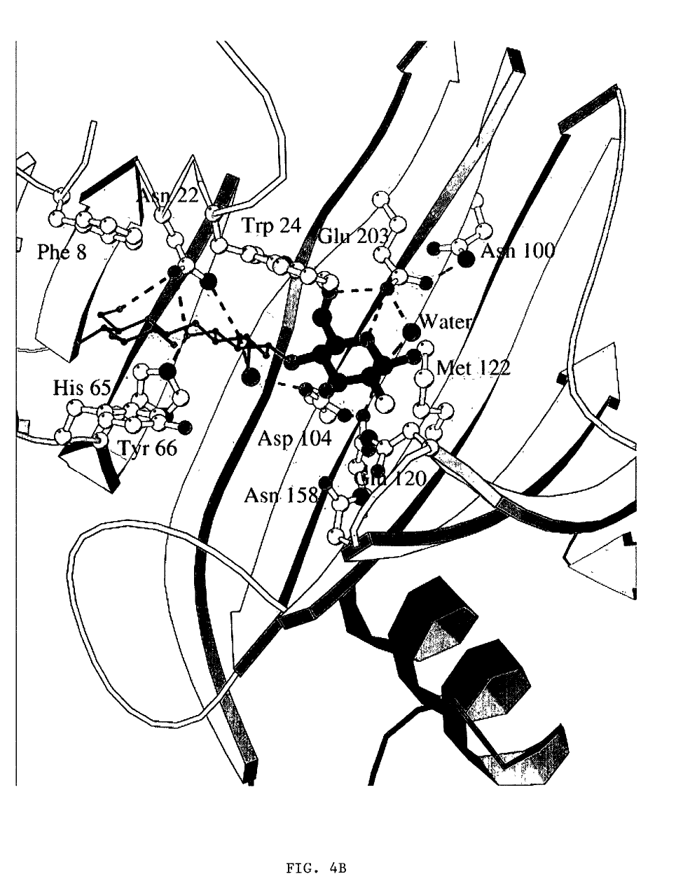

The residues involved in substrate binding (identified by analogy with CelB2) are generally conserved in these more distant binding sites but take up conformations that are not consistently those of the bound state. Stacking interactions with the −3 saccharide are predicted to be provided by Trp 9 and 68, with Asn 24 forming hydrogen bonds with hydroxyls from both the −3 and −2 sugars. The conserved water molecule thought to be crucial for substrate-enzyme interaction in CelB2 has a counterpart in Cel12A, and is held in place through hydrogen bonding with Asp 106, Trp 108 and Glu 203 (CelB2: Asp 104, Trp 106 and Gln 199, the latter two not shown in FIG. 4 for clarity). The −2 sugar will stack with the conserved Trp 26 and will probably also interact with His 67 as in CelB2, although the side chain will need to rotate slightly.

−1 Subsite

As mentioned above, the conformation of the HEPES molecule in the active site resembles a glucose molecule. The resemblance is sufficient for the residues in this region of the active site to adopt a configuration more similar to the complexed than unliganded CelB2 (Table 3). The distance between the two catalytic residues in the unliganded CelB2 structure is 7 Å, longer than the 5.5 Å usually observed in glycosidases with a retaining mechanism, while in the enzyme-substrate complex, rearrangement of the nucleophile Glu 120 reduces the distance to 5.8 Å. In the Cel12A “native” structure with HEPES in the active site, the distance between oxygen atoms on the two catalytic residues is 5.5 Å, indicating that if there is a conformational change to an active form, it has already taken place, perhaps caused by the presence of HEPES. However, the distance in the less similar TrCel12A is also 5.8 Å so an alternative explanation is that the conformational change might not be necessary in some family 12 members.

After alignment of Cel12A with Cel2B containing the two inhibitor species, it is clear that the HEPES molecule aligne almost exactly with the 2-deoxy-2-fluoro-β-D-cellotriose product, and the Cel12A catalytic residues adopt the “product” configuration of the CelB2 residues rather than those of the native (1n1r), or covalent intermediate (FIG. 4). The similarity of HEPES to a glucose molecule is particularly strong in the region of the general acid/base Glu 207, which in CelB2 interacts with the 06 hydroxyl, mimicked by the O8 hydroxyl of HEPES. Once the glucose analogy was revealed, it became clear that HEPES also occupied the site in a mixture of conformations, a residual 3σ peak in the final Fo-Fc map appearing at the end of the nucleophile Glu 124, which might be explained by a covalent intermediate as seen in CelB2 (FIG. 3). However the resolution of the structure and level of HEPES substitution is not sufficient to resolve any minor contributions to the structure.

Most amino acids in this central −1 subsite are conserved or conservatively substituted in the three enzymes. Trp 26 may interact with the O6 hydroxyl of the central sugar, as is the case with Trp 24 in CelB2 (TrCel12A Trp 22). The acid/base Glu 207 (CelB2 203, TrCel12A 200) is flanked by Asn 102 (100, 95) while Asp 106 (104, 99) forms a hydrogen bond with the nucleophile Glu 124 (120, 116). In the CelB2 intermediate complex a conserved water molecule lies ready to carry out nucleophilic attack; a similar water is found in the Cel12A complex with HEPES, but not in TrCel12A, which may be an indication that the active state conformation of the Cel12A enzyme is induced by the presence of HEPES. However the interactions of O2 of the central sugar with amino acids in Cel12A will differ from those in CelB2, due to the differences in sequence in the B8-B7 loop.

Mobile Loop Interactions

The region where the active site cleft of Cel12A differs most markedly from that of CelB2 is in the part of subsite −1 bordered by the loop connecting β-strands B7 and B8 (residues 153-158). In CelB2 Gly153-Asn 158 is described as the ‘mobile’ loop, due to high temperature factors, and is predominantly hydrophilic in sequence (FIG. 2). However, in Cel12A this stretch is replaced by alternating aromatic and hydrophilic amino acids and this exchange and consequent stabilization may be an important contributor to the thermostability of the enzyme (see below). In CelB2, two important interactions involve this loop and might be disrupted by the substitution in Cel12A. Asn 155 is 2.8 Å from the 2-F of the inhibitor, while Asn 158 holds the conformation of the nucleophile Glu 120. Substitution of Asn 158 by Trp 161 in Cel12A does not destroy the interaction between the loop and the nucleophile as the Nε fulfils that role and superimposes almost exactly on the CelB2 Asn ND2 when the structures are aligned. A tryptophan (159) also replaces Asn 155 (CelB2) in Cel12A, but in this structure the side chain is not in the correct orientation to form hydrogen bonds with the substrate (Asn 155 is not shown in FIG. 4B for clarity). However, at this side of the −1 subsite, HEPES no longer resembles glucose, so any conformational change, including rotation of the tryptophan, might not have been triggered. Elucidation of the compensating interaction will have to await a structure of a complex with a more conventional cellulose analogue.

Reducing End of the Cleft

The addition of a new aromatic cluster close to the centre of the active site may fulfil an additional role. Additional aromatic residues in the −3 subsite have been shown to induce thermophilicity, i.e., retention of activity at high temperatures in family 11 xylanases (Georis et al., 2000) and the “mobile” loop cluster seen in the thermostable cellulases may have a similar function at the other end of the cleft. The additional aromatic residues form an extension of the sugar-binding aromatic continuum to the reducing end of the active site cleft and may enhance substrate binding in subsites +1 or +2. Aromatic residues are involved in substrate binding in the defined subsites −3 to −1 and could be in these reducing subsites as well in the thermophilic enzymes, but the structure of a clan H enzyme complex containing saccharides bound in the reducing end of the cleft has not yet been described.

The conserved Met 126 has been proposed to undergo hydrophobic stacking with the +1 subsite sugar, and interaction with this sugar mcan be strengthened in Cel12A by an extra stacking interaction with Tyr 163 (Val 160 in CelB2, Val 156 in TrCel12A, Tyr in the other thermostable enzymes). The interactions of the +2 or possible +3 sugar in the region of the flexible “cord” are less readily predicted due to the lack of structural information. The conformation of the cord, which terminates the active site at the reducing end (loop B6-B9), is very similar in all three family 12 cellulase members, and may be more rigid than in the family 11 xylanases.

Thermostability

Cel12A is an extremely thermostable enzyme, retaining 75% of its activity after 8 hours at 90° C., while CelB2 and TrCel12A are mesophilic; therefore, the Cel12A forms the first thermophilic glycosyl hydrolase family 12 structure to have been determined. From sequence comparison, Cel12A shares the highest sequence identity, up to 39%, with the mesophilic Streptomyces family 12 cellulases to which CelB2 belongs. The level of similarity with the enzymes from thermophilic enzymes such as those from Thermotoga (Thermotoga neapolitana B in FIG. 2) and Pyrococcus furiosus, is lower, which may make more apparent the thermostabilizing features present across both Cel12A and the other thermophiles but absent in the Streptomyces.

Extensive research has been carried into thermostability in many other protein families and between whole genomes (Kumar, S. et al., 2000, Szilágyi & Závodszky 2000, Sterner & Liebl 2001, Vielle & Zeikus 2001). Conclusions of these reviews are that no single feature appears to stabilize every family, and the mechanism of stabilization may depend on the Tₒₚₜ; hyperthermophilic proteins such as Cel12A appear to have different stabilization mechanisms to those with Tₒₚₜ less than 80° C. (Szilagyi & Zavodszky 2000, Vieille & Zeikus 2001). In all these studies the feature that most often correlates with improved thermostability is an increase in electrostatic interactions. Although folding is driven by hydrophobic interactions, electrostatic interactions as a means of stabilizing the folded state become increasingly favourable at higher temperatures (Vieille & Zeikus 2001). Other common features include changes in the amino acid composition, which correlates with increased rigidity at high temperatures, for instance increased numbers of prolines, or a decrease in the glycine content. Those residues that degrade at higher temperatures (Asn, Gln, Cys), or facilitate that degradation (Ser, Thr), are often less abundant in thermophilic enzymes (Kumar et al., 2000, Sterner & Liebl 2001, Vieille & Zeikus 2001). A final observation is that the proportion of ordered secondary structure, particularly a-helices, tends to increase in thermophilic structures. A comparison of these features in the three cellulase structures is given in Table 2.

Ion Pairs

These cellulases are no exception to the trend of increasing electrostatic interactions with Tₒₚₜ; using a strict 4 Å cut-off, 12 ion pairs are identified in Cel12A whereas there are only 4 in both CelB2 and TrCel12A. No ion pair networks were revealed until weaker salt bridges were included, when three three-residue networks appeared in both CelB2 and Cel12A (TrCel12A has none), but unlike either mesophilic enzyme Cel12A also has three longer networks (one each of 4, 5 and 6-members). Ion pairs are clearly an area of significant difference between Cel12A and the mesophilic structures, so they represent a potentially important factor in the thermostability of Cel12A.

Amino Acid Composition

As seen in previous comparisons, the number of uncharged polar residues, which contribute to chemical degradation (Asn, Glu, Ser, Thr), decrease in Cel12A relative to the mesophilic enzymes. However, other reported differences are not observed, for instance both Cel12A and CelB2 have two topologically identical disulphide bonds, similar numbers of glycine residues, and the number of proline residues is actually less in the more thermophilic Cel12A than in CelB2. Thus changes in composition do not seem to be stabilizing directly, but merely protecting against deamidation at high temperatures.

Polar Surface

The extra salt bridges are almost exclusively found on the surface of Cel12A. This increase in ion pairs on the surface is also revealed in the increase of polar surface on the thermophilic enzyme. 76% of the surface of Cel12A was identified by GRASP as being either polar or charged, compared to 73% of TrCel12A and 70% of CelB2. This increase in polarity (and thus decrease in hydrophobic surface) has been shown to correlate with thermostability in a number of systems including the xylanases (McCarthy et al., 2000), where the most thermostable enzyme had 83% polar surface, an even larger increase. This xylanase has a temperature optimum of 75° C., considerably lower than that of Cel12A (more than 90° C.), so if a linear increase of surface polarity with Tₒₚₜ were the rule, the surface polarity of Cel12A might have been expected to be greater. However, a recent survey has shown that extreme thermophiles display a less marked increase in surface polarity over their mesophilic counterparts than moderate thermophiles (Szilágyi and Závodszky, 2000), and the slight increase in the surface polarity of Cel12A fits this trend.

Aromatic Clusters

Another feature identified as being important by Vieille & Zeikus 2001 is an increase in aromatic interactions. In the cellulases the majority of aromatic residues are conserved or subject to conservative substitution between the three structures. Four residues in CelB2 (Phe 93, Phe 125, Trp 172, Phe 174) were identified as being replaced by non-aromatics in Cel12A (Pro 95, Leu 129, Val 175, Leu 178). These residues are all between the two sheets, consolidating the hydrophobic core of the molecule, and the role of the Cel12A non-aromatic residues is probably similar. Nine aromatic residues in TrCel12A are substituted in both of the two bacterial cellulases, seven of which (Phe 10, Phe 30, Trp 48, Tyr 115, Tyr 124, Tyr 185 and Tyr 195) extend the internal aromatic clusters and are mostly aliphatic in Cel12A (Arg 8, Ala 31, Ala 48, Ala 123, Asn 132, Ile 193, Val 202) with the other two, Tyr 150 and Tyr 178 pointing out to the surface and thus being exchanged for polar residues in the thermophilic Cel12A (Asp 158, Asp 185). Cel12A also has three extra aromatic residues involved in internal packing (Phe 64, Tyr 119, Trp 131) but of their counterparts in CelB2 (Asn 62, Asn 117, Arg 127) and TrCel12A (Ile 62, Gly 116, Lys 123), only two at most are able to contribute to the hydrophobic core packing. Tyr 119 is also involved in cavity filling (see above). Thus an increase in aromatic-aromatic interactions does not seem to be an overall stabilizing device. However, as well as the aromatic amino acids involved in core packing, Cel12A has five extra aromatic residues involved in stabilization of the CelB2 “mobile loop”.

Mobile Loop Stabilization

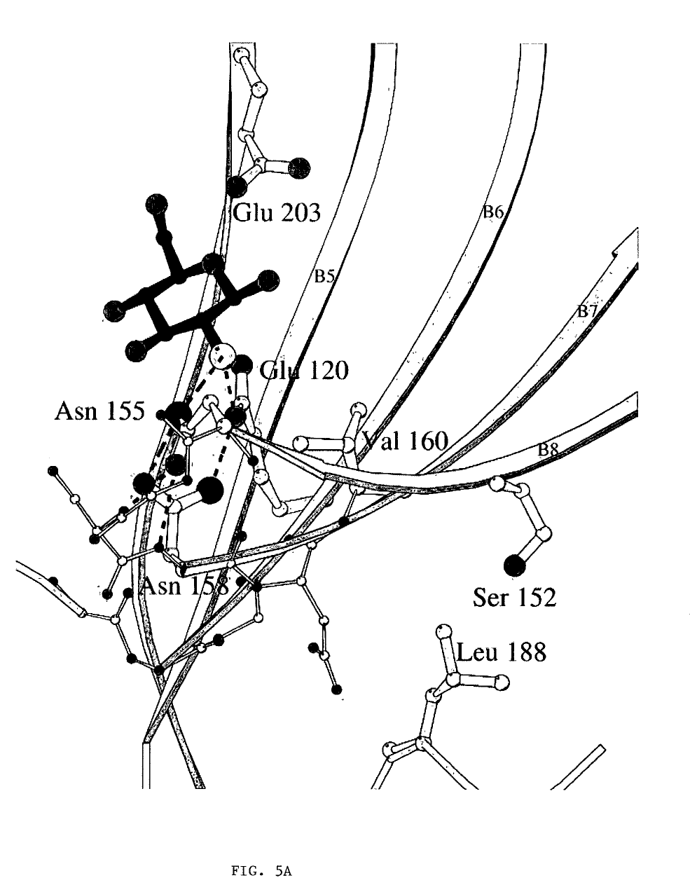

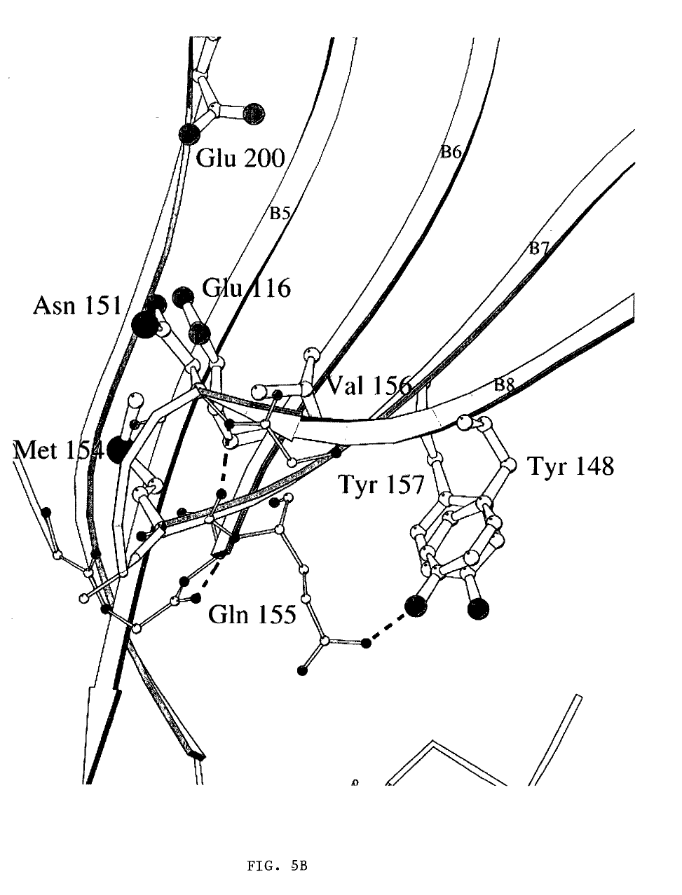

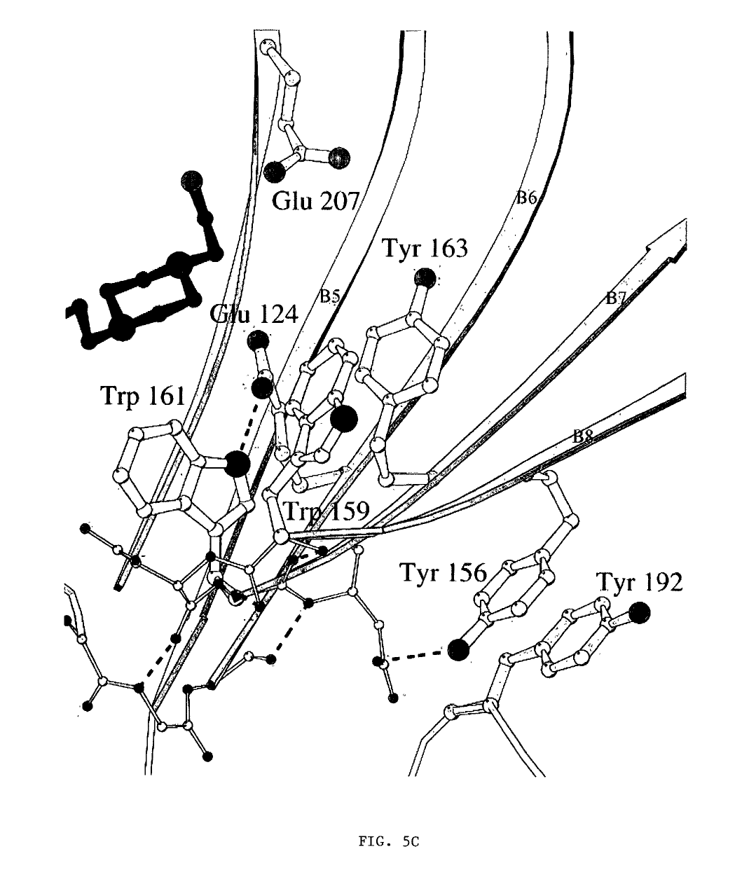

In the CelB2 structure the loop Gly153-Asn 158 between strands B7 and B8 has discontinuous density and high main-chain temperature factors in the native structure (1n1r). With a substrate analogue bound, the temperature factors in this region decrease to merely twice the average main chain value (2n1r), which may be an indication of a conformational change on substrate binding. Such a mobile region, close to the active site, would become an increasing liability at increased temperatures and could form an initiation site for thermal unfolding of the protein. Interactions between this loop, the neighbouring residues and the 2-deoxy-2-fluoro-cellotriose compound are shown in FIG. 5A. TrCel12A (FIG. 5B) has a similar loop composition, with temperature factors lower than that of CelB2, but still above average.

In Cel12A, and indeed by sequence alignment in other thermostable family 12 cellulases from Thermotoga neapolitana (Bok et al., 1998), Thermotoga maritima (Liebl et al., 1996) and Pyrococcus furiosus (Bauer et al., 1999), this region is replaced by a loop of very different character (FIG. 5C). Clearly it is no longer mobile, the loop's main chain temperature factors (between 23 ∈² and 29 Å²) are less than 1.5 times the average, and not greater than those of any other loop in the structure. There are a number of features contributing to this stabilization. In Cel12A the loop between B7 and B8 (residues 157-161) has a single residue deletion, compared to either mesophilic sequence, making the structure more compact. The side chain character alternates between polar and aromatic, rather than being exclusively polar, and for such amphiphilic stretches of sequence it is more energetically favourable to lie on the enzyme surface than be completely water-solvated. Three extra aromatic residues, (Trp 159, Trp 161, Tyr 163), not present in CelB2, TrCel12A or other mesophilic family 12 cellulases, pack together underneath the loop with Trp 108 and extend the active site aromatic cluster. Trp 161, at the centre of the loop, also forms a strong hydrogen bond with Glu 124, the nucleophile. This is similar to the interaction in Cel2B between Asn 158, which is topologically equivalent to Trp 161, and the nucleophile Glu 120, so this interaction is preserved despite the altered environment. The proximity of new aromatic clusters to the active site may have the additional benefit of improving thermophilicity, as shown in a recent study of a family 11 xylanase (Georis et al., 2000). This is supported by our finding that the other thermostable family 12 enzymes also have a tyrosine at position 163 (Cel12A-numbering; FIG. 5C), a position previously proposed to be occupied only by a small residue (Val or Thr; Sandgren et al., 2001).

At the other end of the ‘mobile’ loop, another new aromatic cluster is introduced, between Tyr 156 and Tyr 192 (corresponding residues in CelB2 are Ser 152 and Leu 188). This serves both to tie down the “mobile” loop to the bulk structure and also to form a second new surface-exposed aromatic cluster, which have been shown to increase thermostability in several systems (Kannan & Vishveshwara 2000). In TrCel12A, Tyr 148 corresponds to Tyr156 in Cel12A, hydrogen bonds with Gln 155 (Cel12A Asn 162) and is part of a cluster (FIG. 5B and 5C). However, the aromatic residue with which Tyr 148 forms a stacking interaction, Tyr 157, lies on the other side of the mobile loop (strand B7), i.e., within the same sheet, whereas Tyr 192 in Cel12A follows the helix and is part of the outer side of the molecule, so this cluster is an additional inter-sheet interaction.

Finally the B7-B8 “mobile” loop is further stabilized in Cel12A by main chain hydrogen bonding with the neighbouring loop between B5 and B6. Unusual for a thermostable protein, this loop is longer than in the mesophilic counterparts CelB2 and TrCel12A. This extra length allows the formation of an additional strong (2.90 Å) hydrogen bond between the B5-B6 and the B7-B8 loop in Cel12A (FIG. 5C). In the CelB2 and TrCel12A structures the corresponding distances are 5 Å and 6 Å respectively, so the increased B5-B6 length in Cel12A aids the tethering of the mobile B7-B8 loop, a benefit that must outweigh the cost of introducing flexibility into the B5-B6 loop.

Thus, the addition of the three residue aromatic cluster within the loop and the two residue cluster at the base, together with the insertion in the B5-B6 loop, has stabilized this “mobile” loop. Loop anchoring by hydrogen bonding and hydrophobic interaction has been identified as important for hyperthermophiles (Vieille & Zeikus 2001), and a similar loop stabilization by extra hydrogen bonding and extended aromatic core occurs in the highly thermostable Dictyoglomus thermophilum family 11 xylanase. In the latter case, homologous to the family 12 cellulases, removal of this potential unfolding ‘hot spot’ is postulated to be a major contributor to the thermostability of this enzyme (McCarthy et al., 2000).

Other Possible Thermostabilizing Features

A 10% increase (normalised to sequence length) in the number of hydrogen bonds between Cel12A and the mesophilic CelB2 and TrCel12A was identified through simple distance criteria, but this could simply be a result of the increased percentage of charged residues in the thermophile.

Unlike many systems studied previously, there does not seem to be a significant increase in secondary structure in Cel12A. The Cel12A structure has comparable amounts of α-helical structure, and 3% more sheet structure than the bacterial CelB2, but in comparison to TrCel12A, this can be seen to be irrelevant for thermostability in this system.

Increased compactness and a reduction in loop length have been implicated in thermostability in some systems (Thompson & Eisenberg 1999, Sterner & Liebl 2001), but no large cavities were identified by VOIDOO in any of the cellulases, although those that were found were larger in the mesophilic enzymes than in Cel12A. The cavity found in CelB2 contained 5 water molecules and was between Trp106, which is part of the −1 subsite, and the B5-B6 loop. This cavity is completely filled in Cel12A by Tyr119 (Asn 117) and Trp 68 (Tyr 66), which form a new aromatic cluster with Trp108 (Trp 106), further stabilizing this region of the active site (in TrCel12A this cavity is filled by the B5-B6 loop, which takes up a different conformation). The cavity identified in TrCel12A is spatially close to that in CelB2, but lies in the core of the protein, directly below the nucleophile Glu 116 (Glu 124 in Cel12A). This cavity is filled in Cel12A and CelB2 by contributions from a number of hydrophobic side chains that are more bulky than their counterparts in TrCel12A, rather than any single substitution. The small cavity identified in Cel12A (containing a single water molecule) is in a distant region of the structure, and is caused by an amino acid insertion (Leu 38) in the A2-A3 loop, relative to the CelB2 sequence. Cavity filling would appear not to be a major factor in the thermostability of Cel12A, but cavities in two separate areas of the active site region of the mesophilic proteins have been stabilized.

Comparison with glycosyl hydrolase Family 11 xylanases.

Hydrophobic cluster analysis has indicated significant structural similarity between the xylanases of glycosyl hydrolase family 11, confirmed by the S. lividans CelB structure and examined in detail by Sandgren et al, (2001) in their discussion of the TrCel12A structure. The major area of difference between the two structures is the area identified as being responsible for xylan selectivity, the xylanase “thumb” (Sulzenbacher et al, 1999), which is a long extension to the B7-B8 loop seen in all xylanase structures to date. This corresponds to the “mobile loop” in CelB2 and the different sequence in this region of Cel12A may also alter the specificity of the Rhodothermus enzyme compared to the mesophilic cellulases.

There have been many investigations into the mechanism of action and the thermostability of family 11 xylanases, (Harris et al., 1997, Gruber et al., 1998, Kumar et al., 2000, McCarthy et al., 2000), but the structure of Cel12A provides the first opportunity to compare the basis of thermostability with that in the topologically-similar family 12.

Thermostability

The structures of several thermostable family 11 xylanases have been determined and a number of features identified as being responsible for improved thermostability in comparison with mesophilic structures, although no single feature was identified in every case. In Bacillus D3 (Tₒₚₜ 75° C., Harris et al., 1997), surface aromatic sticky patches were thought responsible for thermostability. In Thermomyces lanuginosus xylanase (Tₒₚₜ 70° C.; Gruber et al., 1998), thermostability was induced by an extra disulphide bond together with an increase in charged residues while in Dictyoglomus thermophilum xylanase (Tₒₚₜ 75° C., McCarthy et al., 2000) an increase in % polar surface together with a longer C-terminal strand were responsible. A 10° C. increase in Tₒₚₜ was seen when an additional aromatic pair was placed at the periphery of the active site of Streptomyces sp. S38 xylanase (Georis et al, 2000), extending the aromatic continuum in the active site and possibly improving substrate binding at high temperature. An analysis of thermostability in family 11 xylanases was undertaken by Kumar et al. (2000), who in their structure of Paecilomyces varioti Bainier xylanase identified the additional disulphide bond, but also other interactions in the vicinity of the active site that could reduce thermal instability. Increases in other features such as buried water molecules, additional ion pairs and aromatic interactions were identified as being locally important.

Many of these features are also found in the thermostable Cel12A compared with the mesophilic members of the family 12 cellulases. There are additional stabilizing interactions close to the centre of the Cel12A active site, both in mobile loop stabilization and cavity filling. Two extra surface-exposed aromatic clusters are introduced in the mobile loop and these may also act as “sticky patches”. The percentage of polar surface increases in the cellulase, as does the length of the C-terminal strand (although the latter may be an artefact of the C-terminal linker used to attach the His tag). The disulphide bond that joins the cord to the helix in many thermophilic xylanases and appears to be one of the primary determinants of thermostability in these molecules is not present in the sequences of thermophilic cellulases determined to date. In the three cellulase structures the cord, loop B6-B9, has a relatively high sequence similarity (it contains two amino acids conserved throughout the family 12 cellulases, including a proline), and identical conformation (FIG. 4). Thus, it is possibly inherently less flexible than that in the xylanases where the structure is poorly conserved, and therefore, it might not require stabilization by disulphide bond addition in the cellulase. Conversely, a prominent feature that appears to contribute to the thermostability of Cel12A, the increase in ion pairs, is not so apparent in the analyses of family 11 xylanase thermostability. A possible explanation for this is that the temperature optima of the thermophilic xylanases fall in the 70-75° C. range while that of Cel12A is over 90° C. and could be classed as hyperthermophilic. As the number of ion pairs has been shown to increase linearly with Tₒₚₜ (Szilágyi & Závodszky 2000), unambiguous identification of this contribution to xylanase thermostability could necessitate a hyperthermophilic xylanase structure. Due to the temperature dependence of the forces involved in stabilization (Sterner & Liebl 2001), the number of thermostabilizing options open to hyperthermophiles may be restricted, so the differences from mesophiles are larger and more apparent, while at the lower temperatures a multiplicity of other minor contributions may also contribute to thermostability.

Thus the determinants of thermostability in family 11 xylanases and family 12 cellulases are not conserved, but an important feature in both families is the stabilization of mobile regions of structure, the cord and mobile loop respectively.

Conclusions

The structure of R. marinus Cel12A represents the first structure of a thermostable cellulase from glucoside hydrolase family 12. When compared with the structure of mesophilic S. lividans CelB2 in complex with an inhibitor it was revealed that a buffer molecule was acting as a glucose analogue. This may have caused a conformational change to the active conformation, and allowed identification of substrate-binding residues. By comparison with the structures of the mesophilic S. lividans CelB2 and T. reesei Cel12A, the three major features contributing to the increased thermostability appeared to be a large increase in the number of ion pairs and the stabilization of a highly mobile loop on the periphery of the active site, together with sequence changes to counter deamidation. Other features such as an increase in polar surface and number of surface-exposed aromatic clusters could also be important.

### EXAMPLE 2

Determination of Potential Thermostabilizing Modifications of Trichoderma reseii Cel12A through Protein Design.

The analysis in Example 1 above was further extended to use the identified thermostabilizing features in R. marinus Cel12A to propose specific mutations in a second related but less thermostable cellulase in order to increase thermostability of the second cellulase. The T. reseii Cel12A was chosen as a test case for this exercise and serves as a demonstration and non-limiting example of how the information and/or the methods disclosed by the invention can be used for protein design of related enzymes.

The structural coordinates of R. marinus and T. reseii Cel12A were displayed and superimposed using the molecular graphics program O (Jones et al., 1991). Superposition of the structures was done with guidance of the sequence alignment shown in FIG. 2. Building homology models of hybrid and mutant proteins were also done with program O.

Identification of Ion Pairs to Introduce in T. reseii Cel12A

In comparison with T. reseii Cel12A, the R. marinus cellulase shows a much higher number of ion pairs among several potential thermostabilizing structural features as shown in Example 1 above. Together with analysis of the structures of other hyperthermophilic proteins, which identified relatively high abundance of ion pairs as a prominent thermostabilzing feature among very thermostable proteins (Vieille & Zeikus 2001), this stongly suggests that ion pairs contribute significantly to the remarkable stability of the R. marinus cellulase. Introduction of similar ion pairs in related protein such as the T. reesei cellulase would be expected to increase stability. Introduction of surface ion pairs, or otherwise improve coulombic interaction among charged surface groups, would be considered to be a preferable general strategy for thermostabilization of proteins through site-directed mutagenesis. Substitution of suitable side chains on the surface of the protein is more likely to be possible without steric hindrance and other undesirable effects compared to changes in the core of the protein and or of more conserved residues (Sanchez-Ruiz & Makhatadze 2001; Ozawa et al., 2001; Spector et al., 2000; Grimsley et al., 1999; Loladze et al., 1999).

With a cut-off value of 5 Å between closest atoms, 15 ion pairs were identified in the R. marinus cellulase structure (Table 4).

TABLE 4
Ion pairs in R. marinus Cel12A cellulase and the corresponding residues in
T. reesei Cel12A cellulase. Ion pairs in the table are limited to maximum shortest distance
of 5 Å between participating side chains.

| R. marinus Cel12A cellulase | | | | T. reesei Cel12A cellulase | |
|---|---|---|---|---|---|
| | Residues in ion pair | Shortest | C_{β}-C_{β} | Corresponding residues, | C_{β}-C_{β} |
| Ion pair | (SEQ ID x) | distance (Å) | distance (Å) | (SEQ ID x) | distance (Å) |
| 1 | Arg8-Glu29 | 4.6 | 7.9 | Gln6, (Ser25) | 9.7 |
| 2 | Asp10-Arg12 | 3.6 | 7.6 | Ala8, Phe10 | 7.2 |
| 3 | Asp13-Arg20 | 3.1 | 5.3 | Thr11, Thr16 | 4.4 |
| 4 | Arg47-Asp49 | 3.2 | 6.8 | (Asp47), Gln49 | 7.9 |
| 5 | Asp51-Arg100 | 5.0 | 9.2 | (Ser51), Arg93 | 10.5 |
| 6 | Arg79-Glu83 | 3.9 | 5.5 | Arg71, Ser75 | 5.0 |
| 7 | Arg80-Glu83 | 4.9 | 4.4 | Thr72, Ser75 | 4.0 |
| 8 | Asp86-Arg88 | 2.9 | 6.3 | Ser78, Pro80 | 5.3 |
| 9 | Arg88-Asp179 | 4.4 | 6.6 | Pro80, Asp171 | 5.6 |
| 10 | Arg100-Glu210 | 3.2 | 4.1 | Arg93, Thr203 | 4.7 |
| 11 | Arg141-Glu153 | 2.9 | 5.4 | Ser133, Thr145 | 5.8 |
| 12 | Glu153-Arg167 | 3.4 | 4.3 | Thr145, Val160 | 4.4 |
| 13 | Lys181-Asp185 | 2.7 | 5.9 | Lys173, Asn177 | 6.0 |
| 14 | Asp186-Arg190 | 2.7 | 6.6 | Tyr178, Lys183 | 7.6 |
| 15 | Arg194-Glu196 | 3.6 | 5.1 | Asn186, Gly189 | — |

The ion pairs are roughly located in two large areas on opposite sides of the molecule on both sides of the active side cleft. There are other potential ion pairs in the R. marinus celllulase besides the ones shown in Table 4. Possible additional ion pairs identified through the analysis are several ion pairs with a distance between 5 Å and 8 Å: Glu4-Arg47 (ion pair 16), Asp10-Arg20 (ion pair 17), Glu35-R216 (ion pair 18), Arg80-Glu196 (ion pair 19), Arg88-Glu177 (ion pair 20), Asp179 and Lys181 (ion pair 21) and Arg216-Asp219 (ion pair 22). Some of these ion pairs are parts of networks of bonds formed between several participating residues at the surface of the protein, such as the one involving Arg194, Glu196, Gln82, Arg80, Glu83, Arg79 and possibly also Lys226. In this region, Glu196 is conserved among the three thermophilic sequences and Arg80 and Glu83 have conservative substitutions so this network may be conserved to some degree among the thermostable cellulases.

In addition to bonds formed between Arg or Lys residues and Glu or Asp residues, other possible ionic bonds were taken into account. Ionic bonds can involve terminal carboxyl or amino groups, which have pKₐ values of about 3.1 and 8.0, respectively, and should therefore normally be charged. The R. marinus cellulase has one bond of this kind, between the amino group of Thr2 and the side chain of Glu39 (ion pair 23, shortest distance 6.26 Å between atoms N and OE1) assuming that the initiating Met1 residue is missing in the crystallized protein. Furthermore, a His residue side chain has a typical pKₐ of 6.5 but a negatively charged side chain in its vicinity can raise its pKₐ and His residues can thus form ionic bond with a neighboring Asp or Glu residue. There is one such bond in R. marinus cellulase, between H67 and E203 (ion pair 24, shortest distance 2.64 Å).

To choose the most promising ion pair candidates for introduction in T. reesei cellulase, the superposition of the structures was used to analyze the corresponding regions in the two structures and to model potential mutations. To maximize probability of a successful introduction of ion pairs through mutations, several features have to be analyzed through the structural comparison and certain criteria met by the potential residues to be mutated. Preferably, the local structure around the site of a particular ion pair in the R. marinus cellulase structure has to be similar to the corresponding site in the protein to be modified. The potential mutated residues should preferably have relative location and conformations similar to the location and conformations of the residues forming the ion pair in the R. marinus cellulase structure. This includes similar distance between C_{β} atom positions and similar angle between the C_{α}-C_{β} bonds of the participating residues. Introduction of residues to form ion pairs has to be possible without steric hindrance and the mutation should not change a residue having important specific structural or functional role. Furthermore, ion pairs that are non-local, i.e., far in sequence and linking distinct secondary elements, are preferable over local ion pairs although local stabilization of loops could also be important. Based on the analysis of the structural comparison between the R. marinus cellulase and the T. reesei cellulase with respect to these criteria, seven ion pairs were identified as most promising for introduction in T. reesei cellulase in order to increase its thermostability. These ion pairs correspond to pairs numbered 3, 6, 9, 10, 11, 12 and 14 in Table 4. Since some residues are conserved (Table 4) or participate in ionic networks, only ten mutations would be needed to introduce the 7 corresponding ion pairs in T. reesei cellulase. Accordingly, these mutations are (grouped in 7 groups, one for each ion pair introduced): Threonine at position 11 to Aspartic acid (Thr11Asp) and Thr16Arg (ion pair 3); Ser 75Glu (ion pair 6); Pro80Arg (ion pair 9); Thr203Glu (ion pair 10); Ser133Arg and Thr145Glu (ion pair 11); Val160Arg (alternatively Lys123Arg) (ion pair 12); Tyr178Asp and Lys183Arg (ion pair 14); residue numbering according to SEQ ID NO: 2. In R. marinus cellulase the ion pairs numbered 11 and 12 form an ionic network with three participating residues (Arg141, Glu153 and Arg167). This network seems to be conserved in all the 3 thermophilic members in the cellulase family alignment shown in FIGS. 2A and 2B. On the contrary, this network seems absent in all the mesophilic members of this group indicating the importance of this structural feature for thermostability in the enzymes from the thermophiles. This ionic network is formed at the base of a loop (“mobile loop”) and could serve to stabilize the loop.

Additional ion pairs can also be readily introduced in T. reesei cellulase according to their presence in a substantially similar regions in the R. marinus cellulase (SEQ ID NO: 1) including Asp10-Arg12 (ion pair 2 in Table 4), Arg80-Glu83 (ion pair 7), Asp86-Arg88 (ion pair 8), Lys181-Asp185 (ion pair 13) and Arg194-Glu196 (ion pair 15), Asp10-Arg20 (ion pair 17), Glu35-Arg216 (ion pair 18), Arg80-Glu196 (ion pair 19), Arg88-Glu177 (ion pair 20) and Arg219-Asp219 (ion pair 22). The corresponding mutations that have to be made to incorporate these ion pairs in the T. reesei protein (SEQ ID NO: 2) are: Ala8Asp and Phe10Arg (ion pair 2), Thr72Arg and Ser75Glu (ion pair 7), Ser78Asp and Pro80Arg (ion pair 8), Asn177Asp (ion pair 13), Asn186Arg and Gly189Glu (ion pair 15), Ala8Asp and Thr16Arg (ion pair 17), Thr34Glu and Asn209Arg (ion pair 18), Thr72Arg and Gly189Glu (ion pair 19), Pro80Arg and Ser169Asp (ion pair 20) and Asn209Arg and Ser212Asp (ion pair 22). Some of the introduced residues could become part of ionic networks and further strengthen other introduced bonds. The bond introduced by the mutations Thr72Arg and Gly189Glu is probably conserved in the thermophilic species in the family.

Residues corresponding to polar but uncharged residues that participate in formation of network of bonds in the R. marinus structure, such as Gln82, can be introduced in the T. reesei enzyme. For example, a residue corresponding to Gln82 in R. marinus could be introduced by the mutation Asn74Gln in T. reseei. Otherwise, a charged residue could also be introduced at this position and could participate equally well in formation of network of bonds.

Mitchinson & Wendt (U.S. Pat. No. 6,268,328), have, from sequence alignment analysis, listed specific substitutions that potentially could alter the thermostability in this family of proteins, such as for the Trichoderma reesei cellulase. The list of sequence locations partially aligns with the location of residues involved in formation of ion bonds in the Rhodothermus marinus cellulase. However, the prediction of formation of ionic bonds was not made for any of the specific modifications and only one combination, Ser133Asp and Thr145Lys (from the groups of alternatives Ser 133 (Gln/Asp/Thr/Phe) and Thr 145 (Asn/Lys/Ser/Asp) of the suggested modifications according to the Trichoderma reeesei sequence, SEQ ID NO: 2), could potentially introduce ion pair corresponding to one of the identified ion pairs in the Rhodothermus marinus cellulase (Arg 141-Glu 153).

Charge-Dipole Interaction and Helix Stabilization

The single α-helix in the R. marinus cellulase contains two of the previously identified ion pairs (Asp 186-Arg 190 and Lys 181-Asp 185). The helix is further stabilized through ionic interactions with the helix dipole. At the N-terminal end of the helix, Asp 179 is about 3.2 Å away from the NH groups of both Lys 181 and Ala 182, thus interacting with the positive end of the helix dipole. This interaction is further strengthened through formation of a network of bonds involving ion pairs Arg 88-Asp 179, Asp 86-Arg 88 and Arg 88-Glu 172. Asp 179 is rather well conserved and present in the T. reesei cellulase where, however, the more extensive network of charge-charge interactions is not conserved.

Two positively charged side chains, of Arg 190 and Arg 194, also surround the positive C-terminal end of the helix in the R. marinus structure. These Arg residues also interact with other positive charges through interactions with the side chains of Asp 186 and Glu 196.

Similar stabilizations by interaction with the dipole of the corresponding helix may be obtained in structurally related proteins through introduction of residues corresponding to Asp 179 or Arg 194.

Loop Modifications

A specific loop is likely to be rather unstable in the mesophilic cellulases from T. reesei and S. lividans as indicated by temperature factor in the determined crystal structures. As outline in Example 1 above, the corresponding loop in the R. marinus cellulase is more stable and contains features conserved also in the Thermotoga and Pyrococcus enzymes as shown in FIGS. 2A and 2B. The specific features of the loop conserved among the thermostable proteins are likely to be important for thermostability and engineering the T. reesei cellulase and other related mesophilic glycosyl hydrolases to include a modified “thermophilic version” of the loop might thus be expected to increase its thermostability. A structural model of a hybrid molecule was constructed consisting of the structure of the T. reesei protein together with the particular loop replaced by the corresponding loop in the R. marinus cellulase. This corresponds to residues 149 to 156 in SEQ ID NO: 2 of the T. reesei structure being replaced by residues 157 to 163 in SEQ ID NO: 1 of the R. marinus cellulase. The modification compared to the mesophilic enzyme includes a smaller loop and three aromatic residues not found in the T. reesei enzyme. Analysis of the model of the hybrid indicated possible steric hindrance preventing the conformation of loop adopted in the thermostable protein. To avoid steric hindrance, two additional mutations were made to the model: Isoleucine 130 to Glycine (Ile130Gly) and Serine 158 to Alanine (Ser158Ala) corresponding to Gly 138 and Ala 165 in the R. marinus structure. No additional serious steric hindrance was observed and accordingly, a modified T. reesei cellulase made with the corresponding mutations outlined could adopt a conformation close to the conformation of this model. This kind of modification—creating a mutant cellulase incorporating features conserved among thermophiles in this family—is expected to have enhanced thermostability. However, modifications in this particular loop similar to the ones described here can be complemented by introduction of ion pairs at the base of the loop as indicated in the previous section. As pointed out, the ionic network created in this way is probably also conserved among the three known thermophilic proteins (shown in FIGS. 2A and 2B).

TABLE 5
Sequences.

| | |
|---|---|
| >Rhodothermus marinus Family 12 Endoglucanase 3, Cel12A | |
| MTVELCGRWDARDVAGGRYRVINNVWGAETAQCIEVGLETGNFTITRADHDNGNNV | SEQ ID NO: 1 |
| AAYPATYFGCHWGACTSNSGLPRRVQELSDVRTSWTLTPITTGRWNAAYDIWFSPV | |
| TNSGNGYSGGAELMIWLNWNGGVMPGGSRVATVELAGATWEVWYADWDWNYIAYRR | |
| TTPTTSVSELDLKAFIDDAVARGYIRPEWYLHAVETGFELWEGGAGLRSADFSVTV | |
| Q | |
| >Trichoderma reesei Family 12 Endoglucanase 3, Cel12A | |
| XTSCDQWATFTGNGYTVSNNLWGASAGSGFGCVTAVSLSGGASWHADWQWSGGQNN | SEQ ID NO: 2 |
| VKSYQNSQIAIPQKRTVNSISSMPTTASWSYSGSNIRANVAYDLFTAANPNHVTYS | |
| GDYELMIWLGKYGDIGPTGSSQGTVNVGGQSWTLYYGYNGANQVYSFVAQTNTTNY | |
| SGDVKNFFNYLIRDNKGYNAAGQYVLSYQFGTEPFTGSGTLNVASWTASIN | |

Note: X at position 1 in the crystallized protein is a cyclic pyro-glutamate produced by the cyclization of an N-terminal glutamine.

References

Alfredsson, G. A., Kristjansson J. K., Hjörleifsdottir S. & Stetter K. O. (1988) Rhodothermus marinus, gen. nov., sp. nov., a thermophilic, halophilic bacterium from submarine hot springs in Iceland. J. Gen. Microbiol., 134, 299-306.

Altschul et al. (1997), Gapped BLAST and PSI-BLAST: a new generation of protein database search programs. Nucleic Acids Res., 25:3389-3402.

Aman & Brosius, (1985) “ATG vectors' for regulated high-level expression of cloned genes in Escherichia coli. Gene 40:183-190.

Andrésson Ó.S. & Fridjónsson Ó.H. (1994) The sequence of the single 16S rRna gene of the thermophilic eubacterium Rhodothermus marinus reveals a distant relationship to the group containing Flexibacter, Bacteriodes and Cytophaga species. J. Bacteriol. 176, 6165-6169.

Asselt, E. J. van, Perrakis, A., Kalt, K. H., Lamzin, V. S. & Dijkstra, B. W. (1998) Accelerated X-ray structure elucidation of a 36 kDa muramidase/transglycosylase using wARP. Acta Crystallogr. D54, 58-73.

Bauer, M. W., et al. (1999). An endoglucanase, EglA, from the hyperthermophilic archaeon Pyrococcus furiosus, hydrolyses β1,4 bonds in mixed-linkage (1→3),(1→4)-β-D-Glucans and cellulose. J. Bacteriol., 181, 284-290.

Barton G. J. (1993). ALSCRIPT a tool to format multiple sequence alignments. Prot. Eng. 6, 37-40.

Bok, J. D., Yernool, D. A. & Eveleigh, D. E. (1998). Purification, characterisation and molecular analysis of thermostable cellulases CelA and CelB from Thermotoga neapolitana. Appl. Microbiol. Biotechnol. 64, 4774-4781.

Brunger, A. T., et al.. (1998). Crystallography and NMR system (CNS): A new software system for macromolecular structure determination. Acta Cryst. D54, 905-921. Collaborative Computational Project, Number 4. (1994). The CCP4 Suite: Programs for Protein Crystallography. Acta Crystallogr. D50, 760-763.

Chen, R. (2001) Enzyme engineering: rational redesign versus directed evolution. Trends Biotechnol. 19:13-14.

Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12.

Cowtan, (1994) Joint CCP4 and ESF-EA CBM Newsletter on Protein Crystallography 31:34-38

Davies G. J., Wilson K. S. & Henrissat B. (1997). Nomenclature for sugar-binding sites in glycosyl hydrolases. Biochem. J., 321, 557-559.

De La Fortelle & Bricogne, (1997) Methods Enzymol. 276:472-494.

Fitzgerald, (1988) J. Appl. Crystallogr. 21:273-278.

Fowler T. and Mitchinson C (2001) Mutant EGIII cellulase, DNA encoding such EGIII compostions and methods for obtaining the same. U.S. Pat. No. 6,187,732.

Forster M. J., (2002) Molecular modelling in structural biology. Micron 33:365-384. Georis, J., et al. (2000). An additional aromatic interaction improves the thermostability and thermophilicity of a mesophilic family 11 xylanase: Structural basis and molecular study. Prot. Sci. 9, 466-475.

Gerald, R et al. (1999) Increasing protein stability by altering long-range coulombic interactions. Prot. Sci. 8:1843-1849.

Gruber K., et al. (1998). Thermophilic xylanase from Thermomyces lanuginosus: High resolution X-ray structure and Modeling studies. Biochemistry, 37, 13475-13485.

Halldórsdóttir S., et al. (1998). Cloning, sequencing and overexpression of a Rhodothermus marinus gene encoding a thermostable cellulase of glycosyl hydrolase family 12. Appl. Microbiol. Biotechnol. 49, 277-284.

Harris G. W., et al. (1997). Structural Basis of the Properties of an Industrially Relevant Thermophilic xylanase. Proteins Struc. Funct. Gen., 29, 77-86.

Havel, T. F., & Snow M. E. (1997). Anew method for building protein conformations from sequence alignments with homologous of known structures. J. Mol. Biol. 217:1-7.

Hendrickson, (1991). Determination of macromolecular structures from anomalous diffraction of synchrotron radiation. Science 254:51-58.

Henrissat B. (1991). A classification of glycosyl hydrolases based on amino acid similarities. Biochem. J., 280, 309-316.

Henrissat B. & Bairoch A. (1993). New families in the classification of glycosyl hydrolases based on amino acid similarities. Biochem. J., 293, 781-788.

Henrissat B and Davies G (1997). Structural and sequence-based classification of glycoside hydrolases. Curr. Opin. Struct. Biol. 7:637-644. Jancarik & Kim, (1991) J. Applied Crystallog. 24:409-411.

Jones, T. A., Zou, J. -Y., Cowan, S. W. & Kjeldgaard, M. (1991). Improved methods for building models in electron-density maps and the location of erros in these models. Acta Crystallogr. A47, 110-119.

Karlin et al., (1993) Applications and statistics for multiple high-scoring segments in molecular sequences. Proc. Natl. Acad. Sci. USA, 90:5873-5877.

Kannan, N. & Vishveshwara, S. (2000) Aromatic clusters: a determinant of thermal stability of thermophilic proteins. Prot. Eng. 13, 753-761.

Kleywegt, G. J. & Jones, T. A. (1994). Detection, delineation, measurement and display of cavities in macromolecular structures. Acta Cryst D50, 178-185.

Kleywegt, G. J. (1996). Use of non-crystallographic symmetry in protein structure refinement. Acta Cryst D52, 842-857.

Kumar S., Tsai, C. -P. & Nussinov R. (2000). Factors enhancing protein thermostability. Prot. Eng. 13, 179-191.

Kumar P. R., et al. (2000). The tertiary structure at 1.59 Å resolution and the proposed amino acid sequence of a family-11 xylanase from the thermophilic fungus Paecilomyces varioti Bainier. J. Mol. Biol. 295, 581-593.

Laskowski, R. A., et al. (1993). PROCHECK: A program to check the stereochemical quality of protein structures. J. Appl. Crystallog. 26, 283-291.

Liebl, W., et al. (1996). Analysis of a Thermotoga maritima DNA fragment encoding two similar thermostable cellulases, CelA and CelB, and characterisation of the recombinant enzymes. Microbiology, 142, 2532-2542.

Loladze, V. (1999) Engineering a Thermostable Protein via Optimization of Charge-Charge Interactions on the Protein surface. Biochemistry 38:16419-16423.

McCarthy A. A., et al. (2000), Sructure of XynB, a highly thermostable β1,4-xylanase from Dictyoglomus thermophilum Rt46B.1, at 1.8 Å resolution. Acta Crystallogr. D56, 1367-1375.

McPherson, (1990) Current approaches to macromolecular crystallization. Eur. J. Biochem. 189:1-23.

McPherson (1999) Crystallization of Biological Macromolecules, Cold Spring Harbor Laboratory Press, Cold Spring Harbor, N.Y.

Methods in Enzymology 114 (1985), Diffraction Methods of Biological Macromolecules (Eds. Wyckoff et al., Academic Press, Orlando, Fla.).

Methods in Enzymology 276 (1997), Diffraction Methods of Biological Macromolecules (Eds. Carter & Sweet, Academic Press, NY);

Mielenz J. R. (2001). Ethanol production from biomass: technology and commercialization status. Curr. Opin. Microbiol. 4:324-329.

Mitchinson C. and Wendt D. J. (2001). Variant EGIII-like cellulase compostions. U.S. Pat. No. 6,268,328.

Myers E. W. and Miller W. (1989). Optimal alignments on linear space. Comput. Appl. Biosci. 4: 11-17.

Navasa, J. (1994). AMoRE: an automated package for molecular replacement. Acta Crystallog. A50, 157-163.

Nicholls, A., Sharp, K. A. & Honig, B. (1991). Protein folding and association: Insights from the interfacial and thermodynamic properties of hydrocarbons. Proteins 11, 281-296.

Okada H., et al.(2000). Identification of active site carboxylic residues in Trichoderma reesei endoglucanase Cel12A by site-directed mutagenesis. J. Mole. Catalysis B., 10, 249-255.

Otwinowski, Z. & Minor, W. (1997). Processing of X-ray diffraction data collected in oscillation mode. Meth. Enzymol. 276, (Carter C. W. & Sweet, R. M. eds.), 307-326, Acad. Press.

Ozawa, T. et al. (2001) Thermostabilization by replacement of specific residues with lysine in a Bacillus alkaline cellulase: building a structural model and implications of newly formed double intrahelical salt bridges. Protein eng. 14:501-504.

Painter, T. J. (1983). Algal polysaccharides. In The polysaccharides. 2 ( Aspinall G. O., ed), 195-285, Academic Press, London.

Pearson and Lipman (1988) Improved tools for biological sequence comparison. PNAS, 85:2444-8.

Perrakis, A., Sixma, T. K., Wilson, K. S. & Lamsin, V. S. (1997). wARP: Improvement and extension of crystallographic phases by weighted averaging of multiple refined dummy atomic models. Acta Crystallogr. D53, 448-455.

Ramakrishnan, C. & Ramachandran G. N. (1965) Stereochemical criteria for polypeptide and protein chain conformations. Allowed conformations for a pair of peptide units. Biophys. J., 5, 909-933.

Rossman (Ed.) The Molecular Replacement Method, Gordon & Breach, New York, 1972

Sali, A. & Blundell, T. L. (1993). Comparative protein modelling by satisfaction of spatial restraints. J. Mol. Biol. 234:779-815.

Sanchez, R. & Sali, A. (1997). Advances in comparative protein-structure modelling. Curr. Opin. Struct. Biol. 7:206-214.

Sanchez-Ruiz J. M. and Makhatadze (2001). To charge or not to charge?. Trends Biotechnol. 19: 132-135.

Sandgren, M., et al. (2001). The X-ray Crystal structure of the Trichoderma reesei family 12 endoglucanase 3, Cel12A, at 1.9 Å resolution. J. Mol. Biol. 308, 295-310.

Spector, S. et al. (2000) Rational Modification of Protein Stability by the Mutation of Charged Surface Residues. Biochemistry 39:872-879.

Sterner, R. & Liebl W. (2001). Thermophilic adaptation of proteins. Crit. Rev. Biochem. Mol. Biol. 36, 39-106.

Studier et al., (1990) Methods Enzymol. 185:60-89.

Sulzenbacher G., et al. (1997). The Streptomyces lividans family 12 endoglucanase: Construction of the Catalytic core, expression and X-ray structure at 1.75 Å resolution. Biochem. 36, 16032-16039.

Sulzenbacher G., et al. (1999). The crystal structure of a 2-fluorocellotriosyl complex of the Streptomyces lividans endoglucanase CelB2 at 1.2 Å resolution. Biochem. 38, 4826-4833.

Szilágyi, A. & Závodszky P. (2000). Structural differences between mesophilic, moderately thermophilic and extremely thermophilic protein subunits: results of a comprehensive survey. Structure, 8, 493-504.

Terwilliger & Berendzen, (1999). Automated MAD and MIR structure solution. Acta Crystallogr. 55:849-861.

Thompson M. J. & Eisenberg D. (1999). Transproteomic evidence of a loop-deletion mechanism for enhancing protein thermostability. J. Mol. Biol. 290, 595-604.

Torelli and Robotti (1994) Advance and adam −2 algorithms for the analysis of global similarity between homologous informational sequences. Comput. Appl. Biosci., 10:3-5

Vieille, C. & Zeikus, G. J. (2001). Hyperthermophilic enzymes: Sources, uses and molecular mechanisms for thermostability. Microbiol. Mol. Biol. Rev. 65, 1-43.

Watenpaugh, (1991) Curr. Opin. Struct. Biol. 1: 1012-1015.

Wicher, K. B., et al. (2001). Deletion of a cytotoxic, N-terminal putative signal peptide results in a significant increase in production yields in Escherichia coli and improved specific activity of Cel12A from Rhodothermus marinus. App. Microbiol. Biotech. 55, 578-584.

Wittman, S., et al. (1994). Purification and characterisation of the CelB endoglucanase from Streptomyces lividans 66 and DNA sequence of the encoding gene. Appl. Environ. Microbiol. 60, 1701-1703.

Wolf et al. (Eds.) (1991) Isomorphous Replacement and Anomalous scattering, Science and Engineering Council, Warrington, WA44AD, UK.

Zechel D. L., et al. (1998). Identification of Glu-120 as the catalytic nucleophile in Streptomyces lividans endoglucanase CelB., 336, 139.

What is claimed is:

1. A crystallizable composition comprising a substantially pure protein having at least 50% amino acids sequence identity with amino acid sequence shown in SEQ ID NO: 1.

2. The crystallizable composition of claim 1, wherein said protein is a thermophilic family 12 glycosyl hydrolase.

3. A crystallized molecule or crystallized molecular complex comprising a protein having at least 50% amino acid sequence identity with the amino acid sequence shown is SEQ ID NO: 1.

4. The crystallized molecule or crystallized molecular complex of claim 3, comprising a protein having a β-jelly roll fold.

5. The crystallized molecule or crystallized molecular complex of claim 3, comprising a glycosyl hydrolase having at least 75% amino acid sequence identity with the amino acid sequence shown is SEQ ID NO: 1.

6. The crystallized molecule or crystallized molecular complex of claim 3, comprising a thermophilic family 12 glycosyl hydrolase.

7. The crystallized molecule or crystallized molecular complex according to claim 3, wherein the crystal is characterized by a space group P2₁2₁2₁ and unit cell dimensions of a=56.1 Å, b=67.8 Å, and c=132.3 Å.

8. A machine-readable data storage medium comprising a data storage material encoded with data essentially defining the protein structure of a crystallized molecule or crystallized molecular complex according to claim 3.

9. The machine-readable data storage medium of claim 8, wherein said data essentially defines the protein structure represented by the structure coordinates set forth in FIG. 6.

10. The machine-readable data storage medium of claim 8, wherein the data storage material is encoded with the structure coordinates set forth in FIG. 6, or mathematically related coordinates or other data defining the same structure as said coordinates.

11. A method for modeling the structure of a first protein with at least 40% amino acid sequence identity to the sequence set forth in SEQ ID NO: 1 comprising aligning the sequence of said first protein with the sequence of a reference crystallized protein of claim 3, and incorporating at least a part of the sequence of said first protein into the structure of said reference crystallized protein, thereby creating a structural model of at least a part of said first protein.

12. The method of claim 11 further comprising the steps of
a) subjecting said structural model to energy-minimization, optionally combined with molecular dynamics, to obtain an energy-minimized structural model;
b) optionally remodeling the regions of said structural model or energy-minimized model where geometrical restraints are violated to obtain structure coordinates of a final structural model of said first protein; and
c) optionally modeling regions of said first protein, said structural model or energy-minimized structural model using information of other predetemined structural models.

13. A method for determining the protein structure of a first protein from crystallographic protein structure data that has insufficient phase information for a structure determination, comprising:
a) determining the phase information for said first protein with molecular replacement methods based on an obtained structure of a crystallized protein of claim 3; and
b) determining the protein structure by use of the initial structure data and the obtained phase information.

14. A method for modifying in a structurally defined region a first protein that is related to a crystallized protein of claim 3, comprising the steps of:
a) obtaining a first amino acid sequence of said first protein and a nucleic acid encoding said sequence, and aligning said first sequence with the sequence of said crystallized protein;
b) selecting a region in said first sequence that aligns with a structurally defined region in said crystallized protein, and changing the nucleotide sequence of said nucleic acid in the region that encodes for said region in said first sequence to exchange, add and/or subtract one or more amino acid residues in said region of said first protein; and
c) expressing said modified first protein in a suitable expression system.

15. The method of claim 14, wherein the modification of said first protein increases thermostability.

16. The method of claim 14, wherein the modification comprises a modification in a region of said first sequence that aligns with residues 155-165 of SEQ ID NO: 1, wherein the modification decreases the mobility of said region in said first protein.

17. The method of claim 14, wherein said region of the modified first protein is substantially similar to the region of residues 155-165 of SEQ ID NO: 1.

18. The method of claim 14, wherein the modification comprises having a Gly or Ala residue that alignes with Gly138 of SEQ ID NO: 1.

19. The method of claim 14, wherein the modification comprises having a Gly or Ala residue that alignes with Ala165 of SEQ ID NO: 1.

20. The method of claim 14, wherein the modification increases the ion pair number.

21. The method of claim 14, wherein the modification comprises having a Gln, Asn, Arg, Lys, His, Asp or Glu residue at the sequence location that aligns with Gln82 of SEQ ID NO: 1.

22. The method of claim 14, wherein the modification comprises having an Asp or Glu residue at the sequence location that aligns with Glu 39 of SEQ ID NO: 1 and an N-terminal residue at the sequence location that aligns with Thr 2 of SEQ ID NO: 1

23. The method of claim 14, wherein the modification stabilizes a helix corresponding to residues 180-191 of SEQ ID NO: 1 by having either an Arg, Lys or His residue at the sequence location that aligns with Gln82 of SEQ ID NO: 1; an Asp or Glu residue at the sequence location that aligns with Asp 179 of SEQ ID NO: 1; or both modifications

24. A protein modified by the method of claim 14.

25. A crystallized molecule or molecular complex comprising a protein having a crystal structure comprising structural entities that can be independently superimposed on reference structural entities within the structure defined by the structural coordinates set forth in FIGS. 6A-PPP such that the root mean square deviation of Cα atoms being superimposed is less than 0.8 Å, the reference entities comprising (i) residues 18-26, (ii) residues 31-37, (iii) residues 56-64, (iv) residues 84-95, (v) residues 99-112, (vi) residues 122-142, (vii) residues 149-157, (viii) residues 161-173, (ix) residues 196-210, and (x) residues 215-224 of the protein structure defined by said coordinates of FIGS. 6A-PPP.

26. The crystallized molecule or molecular complex of claim 25, wherein said root mean square deviation of the Cα atoms of said structural entitites when superimposed on said reference entities is less than 0.6 Å.

27. The crystallized molecule or molecular complex of claim 25, comprising a polypeptide having a structure that can be superimposed on the reference protein structure defined by the structural coordinates set forth in FIGS. 6A-PPP such that the root mean square deviation of the Cα atoms of said polypeptide from the Cα atoms of said protein structure is less than 0.8 Å.

28. A machine-readable data storage medium comprising a data storage material encoded with data essentially defining the protein structure of a crystallized molecule or molecular complex according to claim 25.

29. A method of modifying a clan C glycosyl hydrolase wherein the modification comprises one or more modifications selected from the group consisting of:
having an Arg, Lys or His residue at one position and an Asp or Glu residue at a second position, wherein the positions are at sequence locations that align with Glu 4 and Arg 47 of SEQ ID NO: 1, respectively;
having an Arg, Lys or His residue at one position and an Asp or Glu residue at a second position, wherein the positions are at sequence locations that align with Arg 8 and Glu 29 of SEQ ID NO: 1, respectively;
having an Arg, Lys or His residue at one position and an Asp or Glu residue at a second position, wherein the positions are at sequence locations that align with Asp 10 and Arg 12 of SEQ ID NO: 1, respectvely;
having an Arg, Lys or His residue at one position and an Asp or Glu residue at a second position, wherein the positions are at sequence locations that align with Asp 10 and Arg 20 of SEQ ID NO: 1, respectively;
having an Arg, Lys or His residue at one position and an Asp or Glu residue at a second position, wherein the positions are at sequence locations that align with Asp 13 and Arg 20 of SEQ ID NO: 1, respectively;
having an Arg, Lys or His residue at one position and an Asp or Glu residue at a second position, wherein the positions are at sequence locations that align with Glu 35 and Arg 216 of SEQ ID NO: 1, respectively;
having an Arg, Lys or His residue at one position and an Asp or Glu residue at a second position, wherein the positions are at sequence locations that align with Arg 47 and Asp 49 of SEQ ID NO: 1, respectively;
having an Arg, Lys or His residue at one position and an Asp or Glu residue at a second position, wherein the positions are at sequence locations that align with Asp 51 and Arg 100 of SEQ ID NO: 1, respectively;
having an Arg, Lys or His residue at one position and an Asp or Glu residue at a second position, wherein the positions are at sequence locations that align with His 67 and Glu 203 of SEQ ID NO: 1, respectively;
having an Arg, Lys or His residue at one position and an Asp or Glu residue at a second position, wherein the positions are at sequence locations that align with Arg 79 and Glu 83 of SEQ ID NO: 1, respectively;
having an Arg, Lys or His residue at one position and an Asp or Glu residue at a second position, wherein the positions are at sequence locations that align with Arg 80 and Glu 83 of SEQ ID NO: 1, respectively;
having an Arg, Lys or His residue at one position and an Asp or Glu residue at a second position, wherein the positions are at sequence locations that align with Arg 80 and Glu 196 of SEQ ID NO: 1, respectively;
having an Arg, Lys or His residue at one position and an Asp or Glu residue at a second position, wherein the positions are at sequence locations that align with Asp 86 and Arg 88 of SEQ ID NO: 1, respectively;
having an Arg, Lys or His residue at one position and an Asp or Glu residue at a second position, wherein the positions are at sequence locations that align with Arg 88 and Glu 177 of SEQ ID NO: 1, respectively;
having an Arg, Lys or His residue at one position and an Asp or Glu residue at a second position, wherein the positions are at sequence locations that align with Arg 88 and Asp 179 of SEQ ID NO: 1, respectively;
having an Arg, Lys or His residue at one position and an Asp or Glu residue at a second position, wherein the positions are at sequence locations that align with Arg 100 and Glu 210 of SEQ ID NO: 1, respectively;
having an Arg, Lys or His residue at one position and an Asp or Glu residue at a second position, wherein the positions are at sequence locations that align with Arg 141 and Glu 153 of SEQ ID NO: 1, respectively;
having an Arg, Lys or His residue at one position and an Asp or Glu residue at a second position, wherein the positions are at sequence locations that align with Glu 153 and Arg 167 of SEQ ID NO: 1, respectively;
having an Arg, Lys or His residue at one position and an Asp or Glu residue at a second position, wherein the positions are at sequence locations that align with Asp 179 and Lys 181 of SEQ ID NO: 1, respectively;
having an Arg, Lys or His residue at one position and an Asp or Glu residue at a second position, wherein the positions are at sequence locations that align with Lys 181 and Asp 185 of SEQ ID NO: 1, respectively;
having an Arg, Lys or His residue at one position and an Asp or Glu residue at a second position, wherein the positions are at sequence locations that align with Asp 186 and Arg 190 of SEQ ID NO: 1, respectively;
having an Arg, Lys or His residue at one position and an Asp or Glu residue at a second position, wherein the positions are at sequence locations that align with Arg 194 and Glu 196 of SEQ ID NO: 1, respectively;
having an Arg, Lys or His residue at one position and an Asp or Glu residue at a second position, wherein the positions are at sequence locations that align with Arg 216 and Asp 219 of SEQ ID NO: 1.

30. The method of claim 29 wherein the one or more introduced amino acid residues form one or more ionic bonds.

31. An isolated clan C glycosyl hydrolase that comprises one or more substituted residues selected from the group consisting of:
having an Arg, Lys or His residue at one position and an Asp or Glu residue at a second position, wherein the positions are at sequence locations that align with Glu 4 and Arg 47 of SEQ ID NO: 1, respectively;
having an Arg, Lys or His residue at one position and an Asp or Glu residue at a second position, wherein the positions are at sequence locations that align with Arg 8 and Glu 29 of SEQ ID NO: 1, respectively;
having an Arg, Lys or His residue at one position and an Asp or Glu residue at a second position, wherein the positions are at sequence locations that align with Asp 10 and Arg 12 of SEQ ID NO: 1, respectively;
having an Arg, Lys or His residue at one position and an Asp or Glu residue at a second position, wherein the positions are at sequence locations that align with Asp 10 and Arg 20 of SEQ ID NO: 1, respectively;
having an Arg, Lys or His residue at one position and an Asp or Glu residue at a second position, wherein the positions are at sequence locations that, align with Asp 13 and Arg 20 of SEQ ID NO 1, respectively;
having an Arg, Lys or His residue at one position and an Asp or Glu residue at a second position, wherein the positions are at sequence locations that align with Glu 35 and Arg 216 of SEQ ID NO: 1, respectively;
having an Arg, Lys or His residue at one position and an Asp or Glu residue at a second position, wherein the positions are at sequence locations that align with Arg 47 and Asp 49 of SEQ ID NO: 1, respectively;
having an Arg, Lys or His residue at one position and an Asp or Glu residue at a second position, wherein the positions are at sequence locations that align with Asp 51 and Arg 100 of SEQ ID NO: 1, respectively;
having an Arg, Lys or His residue at one position and an Asp or Glu residue at a second position, wherein the positions are at sequence locations that align with His 67 and Glu 203 of SEQ ID NO: 1, respectively;
having an Arg, Lys or His residue at one position and an Asp or Glu residue at a second position, wherein the positions are at sequence locations that align with Arg 79 and Glu 83 of SEQ ID NO: 1, respectively;
having an Arg, Lys or His residue at one position and an Asp or Glu residue at a second position, wherein the positions are at sequence locations that align with Arg 80 and Glu 83 of SEQ ID NO: 1, respectively;
having an Arg, Lys or His residue at one position and an Asp or Glu residue at a second position, wherein the positions are at sequence locations that align with Arg 80 and Glu 196 of SEQ ID NO: 1, respectively;
having an Arg, Lys or His residue at one position and an Asp or Glu residue at a second position, wherein the positions are at sequence locations that align with Asp 86 and Arg 88 of SEQ ID NO: 1, respectively;
having an Arg, Lys or His residue at one position and an Asp or Glu residue at a second position, wherein the positions are at sequence locations that align with Arg 88 and Glu 177 of SEQ ID NO: 1, respectively;
having an Arg, Lys or His residue at one position and an Asp or Glu residue at a second position, wherein the positions are at sequence locations that align with Arg 88 and Asp 179 of SEQ ID NO: 1, respectively;
having an Arg, Lys or His residue at one position and an Asp or Glu residue at a second position, wherein the positions are at sequence locations that align with Arg 100 and Glu 210 of SEQ ID NO: 1, respectively;
having an Arg, Lys or His residue at one position and an Asp or Glu residue at a second position, wherein the positions are at sequence locations that align with Arg 141 and Glu 153 of SEQ ID NO: 1, respectively;
having an Arg, Lys or His residue at one position and an Asp or Glu residue at a second position, wherein the positions are at sequence locations that align with Glu 153 and Arg 167 of SEQ ID NO: 1, respectively;
having an Arg, Lys or His residue at one position and an Asp or Glu residue at a second position, wherein the positions are at sequence locations that align with Asp 179 and Lys 181 of SEQ ID NO: 1, respectively;
having an Arg, Lys or His residue at one position and an Asp or Glu residue at a second position, wherein the positions are at sequence locations that align with Lys 181 and Asp 185 of SEQ ID NO: 1, respectively;
having an Arg, Lys or His residue at one position and an Asp or Glu residue at a second position, wherein the positions are at sequence locations that align with Asp 186 and Arg 190 of SEQ ID NO: 1, respectively;
having an Arg, Lys or His residue at one position and an Asp or Glu residue at a second position, wherein the positions are at sequence locations that align with Arg 194 and Glu 196 of SEQ ID NO: 1, respectively;
having an Arg, Lys or His residue at one position and an Asp or Glu residue at a second position, wherein the positions are at sequence locations that align with Arg 216 and Asp 219 of SEQ ID NO: 1;

32. The protein of claim 31, wherein the protein is a family 12 glycosyl hydrolase.

33. The protein of claim 31, wherein the protein obtainable prior to improvement from a Trichoderma species.

34. A crystallized molecule or molecular complex comprising a family 12 glycosyl hydrolase obtainable from Rhodotermus marinus.


SEQUENCE LISTING (2 sequences)

SEQ ID NO: 1 - protein, 225 aa, Rhodothermus marinus
MTVELCGRWDARDVAGGRYRVINNVWGAETAQCIEVGLETGNFTITRADHDNGNNVAAYPAIYFGCHWGACTSNSGLPRRVQELSDVRTSWTLTPITTGRWNAAYDIWFSPVTNSGNGYSGGAELMIWLNWNGGVMPGGSRVATVELAGATWEVWYADWDWNYIAYRRTTPTTSVSELDLKAFIDDAVARGYIRPEWYLHAVETGFELWEGGAGLRSADFSVTVQ

SEQ ID NO: 2 - protein, 218 aa, Trichoderma reesei
  VARIANT 1: Xaa = Any Amino Acid
XTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN